(19) United States
(12) Patent Application Publication
(10) Pub. No.: US 2024/0423968 A1
(43) Pub. Date: Dec. 26, 2024

(54) USE OF PROTEASOME-TARGETING SMALL MOLECULES TO MITIGATE MUSCLE WASTING

(71) Applicants: The Board of Regents of the University of Texas System, Austin, TX (US); The Board of Trustees of Michigan State University, East Lansing, MI (US)

(72) Inventors: Maria Gaczynska, Hondo, TX (US); Pawel A. Osmulski, Hondo, TX (US); Jetze Tepe, East Lansing, MI (US)

(21) Appl. No.: 18/741,420

(22) Filed: Jun. 12, 2024

Related U.S. Application Data
(60) Provisional application No. 63/472,697, filed on Jun. 13, 2023.

(51) Int. Cl. A61K 31/445; A61K 31/40; A61P 21/00

(52) U.S. Cl. CPC A61K 31/445; A61K 31/40; A61P 21/00

(57) ABSTRACT

The present disclosure relates to compounds that modulate (i.e., regulate) proteasome activity, pharmaceutical compositions containing such compounds, and uses of these compounds and compositions for the treatment of muscle wasting. This abstract is intended as a scanning tool for purposes of searching in the particular art and is not intended to be limiting of the present invention.

### CROSS-REFERENCE TO RELATED APPLICATIONS

This Application claims the benefit of U.S. Application No. 63/472,697, filed on Jun. 13, 2023, the contents of which are incorporated herein by reference in their entirety.

### STATEMENT REGARDING FEDERALLY SPONSORED RESEARCH

This invention was made with government support under grant number AG013319 awarded by the National Institutes of Health. The government has certain rights in the invention.

### BACKGROUND

Balancing the rates of protein synthesis and protein degradation leads to the maintenance of muscle mass. When synthesis exceeds degradation the result is often an increase in the size and performance of muscle fibers. Conversely, when net balance favors protein degradation, muscle size and performance are reduced. (Graham et al. (2021) Am J Physiol Cell Physiol. 321(1): C40-C57). It is this imbalance in skeletal muscle protein synthesis and degradation that leads to muscle wasting. Id.

Muscle wasting, also known as muscle atrophy, can be observed in various diseases (e.g., cancer, diabetes, heart failure, chronic kidney disease, chronic obstructive pulmonary disease) and acute critical illnesses (e.g., burns, sepsis, trauma) (Cohen et al. (2015) Nature Reviews: Drug Discover 14: 58-74). For example, after a serious burn injury, there can be increased protein degradation that is not counterbalanced by protein synthesis, leading to systemic muscle loss and weakness (Clark et al. (2020) Journal of Burn Care & Research 41(1): 33-40). Such effects are common amongst burn patients and negatively effect the hospital course and recovery, resulting in an increased risk of infection and impaired wound healing. Id.

In some cases, muscle wasting can also be observed without any overt disease (e.g., sarcopenia) (Cohen et al. (2015) Nature Reviews: Drug Discover 14: 58-74). Sarcopenia is the age-related progressive loss of muscle mass and strength. Signs and symptoms of sarcopenia include weakness, fatigue, loss of energy, balance problems, and trouble walking and standing. For older patients, this muscle loss or weakness can lead to falls, broken bones, and other serious injuries that can effect a person's ability to care for oneself. Optimal care for people with sarcopenia is essential because the condition has high personal, social, and economic burdens when untreated (Mijnarends et al. (2018) J Nutr Health Aging 22: 766-73).

Another form of muscle wasting is cachexia, a highly debilitating condition characterized by pronounced weight loss, muscle weakness, anemia, insulin resistance, and extreme fatigue (Morley et al. (2006) Am J Clin Nutr 83: 735-43). This disease syndrome is tightly associated with cancer and other chronic diseases such as AIDS, heart failure, and inflammatory bowel disease. In advanced cancers, >50% of patients exhibit cachectic symptoms, and remarkably ˜20% of cancer-related mortalities derive from cachexia rather than direct tumor burden (Tisdale, MJ (2002) Nat Rev Cancer 2: 862-71). This wasting condition also lowers responsiveness to chemotherapy and radiotherapy, contributing to poor prognosis and a depreciating quality of life.

Although therapeutic options against these conditions are still lacking, research in the last two to three decades has made significant progress in elucidating some of the key mediators of muscle wasting. These include immune and tumor-derived cytokines such as tumor necrosis factor α (TNFα; originally termed cachectin), interleukin (IL)-1, IFNγ, and IL-6, as well as tumor-specific factors such as lipid mobilizing factor and the 24-kDa sulfated glycoprotein proteolysis-inducing factor (PIF) (Argiles et al. (2006) Cancer Treat Res 130: 199-217; Tisdale, MJ (2006) Nutr Clin Pract 21: 168-74). However, in spite of this knowledge, the heterogeneity in the expression pattern of these indicators and their potential synergistic mode of action have made the direct targeting of these factors challenging and have yielded little clinical benefit.

Another breakthrough in the field came from the realization that the majority of these cachectic factors regulate skeletal muscle wasting by reducing the rate of protein synthesis at the level of protein translation or RNA content (Rennie et al. (2004) Annu Rev Physiol 66: 799-828) and by stimulating protein catabolism predominantly through the activation of the ATP-dependent ubiquitin-proteasome pathway (Jagoe and Goldberg (2001) Curr Opin Clin Nutr Metab Care 4: 183-90; Tisdale, MJ (2005) Physiology (Bethesda) 20: 340-8). New findings also suggest that of the myofibrillar proteins implicated in mediating muscle atrophy and the wasting state, myosin heavy chain is a preferred substrate that can be inhibited at the RNA level or degraded through a ubiquitin-associated proteolytic process (Archaryya et al. (2004) J Clin Invest 114: 370-8). In addition, there has been significant progress in identifying new signaling pathways that contribute to muscle atrophy that are potentially pertinent in cancer. These include the down-regulated insulin and insulin-like growth factor pathways that lead to Akt inactivation and reversal of muscle hypertrophy, the angiotensin system that operates through the activation of caspases, the transforming growth factor-β family member myostatin, the p38 mitogen-activated protein kinase, Foxo, nuclear factor κB (NF-κB), activator protein-1, and p53 transcription factors, as well as a newly described pathway involving the dystrophin glycoprotein complex (DGC) (Stitt et al. (2004) Mol Cell 14: 395-403; Sandri et al. (2004) Cell 117: 399-412; Song et al. (2005) J Clin Invest 115: 451-8; Lee, S J (2004) Annu Rev Cell Dev Biol 20: 61-86; Cai et al. (2004) Cell 119: 285-98; Costelli et al. (2005) Int J Oncol 26: 1663-8; Acharyya et al. (2005) Cancer Cell 8: 421-32; Schwarzkopf et al. (2006) Genes Dev 20: 3440-52). Interestingly, most, if not all, of these emerging pathways seem to mediate their effects through the activation of the ubiquitin proteasome system. Without wishing to be bound by theory, these findings reinforce that clinical efforts on treating muscle wasting should remain focused on targeting proteasome activity.

Unfortunately, nearly all proteasome agonists developed to date are competitive inhibitors that covalently bind in the catalytic subunits in the β-ring of the proteasome (Groll et al. (2006) J. Am. Chem. Soc. 128: 5136-5141). While useful to treat blood cancers, such inhibitors are toxic for other applications. Thus, there remains a need for compounds that allosterically regulate the proteasome and methods of making and using same. These needs and others are met by the following disclosure.

### SUMMARY

In accordance with the purpose(s) of the invention, as embodied and broadly described herein, the invention, in one aspect, relates to compounds useful in treating or preventing muscle wasting such as, for example, muscle wasting due to human immunodeficiency virus (HIV), chronic renal failure, kidney disease, chronic obstructive pulmonary disease (COPD), multiple sclerosis, cystic fibrosis, rheumatoid arthritis, advanced dementia, cachexia, congestive heart failure (CHF), cancer, a limb injury, and immobilization.

Thus, disclosed are methods of treating muscle wasting in a subject in need thereof, the method comprising administering to the subject an effective amount of a compound having a structure represented by a formula selected from:

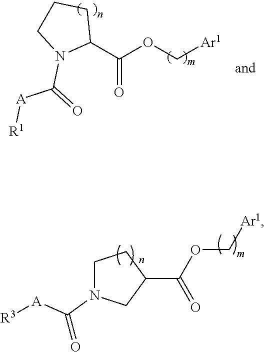

wherein n is 1 or 2; wherein m is 1, 2, or 3; wherein A is selected from —O— and —C(O)—; wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl); wherein q is 1, 2, or 3; wherein each of R^{20a} and R^{20b}, when present, is independently selected from hydrogen and C1-C4 alkyl; wherein Cy¹ is selected from cyclohexyl, a 6-membered heterocycloalkyl, and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; wherein R³ is selected from C1-C8 alkyl, (CH₂)_{q}Cy¹, and Cy¹; and wherein Ar¹ is selected from C6-aryl and pyridinyl, and is substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof, wherein the subject has not been diagnosed as having cancer prior to the administering step.

Also disclosed are methods of treating muscle wasting in a subject having a cancer, the method comprising administering to the subject an effective amount of a compound having a structure represented by a formula selected from:

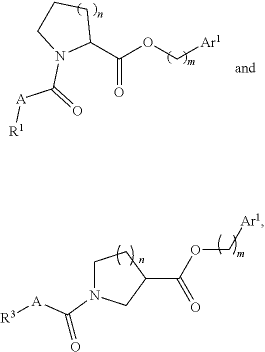

wherein n is 1 or 2; wherein m is 1, 2, or 3; wherein A is selected from —O— and —C(O)—; wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl); wherein q is 1, 2, or 3; wherein each of R^{20a} and R^{20b}, when present, is independently selected from hydrogen and C1-C4 alkyl; wherein Cy¹ is selected from cyclohexyl, a 6-membered heterocycloalkyl, and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; wherein R³ is selected from C1-C8 alkyl, (CH₂)_{q}Cy¹, and Cy¹; and wherein Ar¹ is selected from C6-aryl and pyridinyl, and is substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof, wherein the subject has experienced a weight loss of 5% or more over a time period of 12 months or less prior to the administering step.

Also disclosed are methods of treating muscle wasting in a subject having cancer, the method comprising administering to the subject an effective amount of a compound selected from:

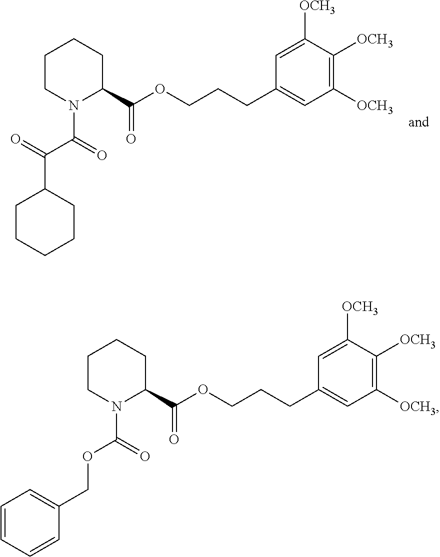 [SMILES: COc1cc(CCCOC(=O)[C@@H]2CCCCN2C(=O)C(=O)C2CCCCC2)cc(OC)c1OC ; COc1cc(CCCOC(=O)[C@@H]2CCCCN2C(=O)OCc2ccccc2)cc(OC)c1OC]

or a pharmaceutically acceptable salt thereof, wherein the cancer is selected from pancreatic cancer and gastric cancer, and wherein the subject has experienced a weight loss of 5% or more over a time period of 6 months or less prior to the administering step.

Also disclosed are kits comprising a compound having a structure represented by a formula selected from:

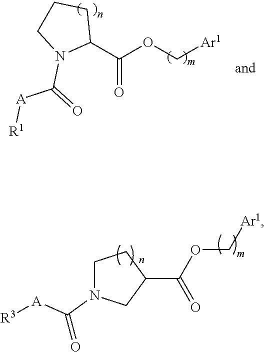

wherein n is 1 or 2; wherein m is 1, 2, or 3; wherein A is selected from —O— and —C(O)—; wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl); wherein q is 1, 2, or 3; wherein each of R^{20a} and R^{20b}, when present, is independently selected from hydrogen and C1-C4 alkyl; wherein Cy¹ is selected from cyclohexyl, a 6-membered heterocycloalkyl, and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; wherein R³ is selected from C1-C8 alkyl, (CH₂)_{q}Cy¹, and Cy¹; and wherein Ar¹ is selected from C6-aryl and pyridinyl, and is substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof, and one or more selected from: (a) an agent known to treat muscle wasting; (b) instructions for administering the compound in connection with treating muscle wasting; and (c) instructions for treating muscle wasting.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a representative schematic of the ubiquitin-proteasome pathway.

FIG. 2 shows a representative schematic of the modulation of the proteasome in the presence of the disclosed small molecule modulators.

FIG. 3 shows representative data illustrating the myotube morphology.

FIG. 4 shows a representative graphical presentation of the myotube morphology.

FIG. 5 shows representative data illustrating the cellular atomic force microscopy of the cell surface topography.

FIG. 6 shows a representative graphical presentation of the cell surface isotropy.

FIG. 7 shows a representative principal component analysis of the AFM-assessed mechanical paramerts of model myotubes.

FIG. 8 shows representative data illustrating 26S activity in lysates.

FIG. 9 shows representative data illustrating the levels of autophagy related protein 7 (ATG7) in lysates.

FIG. 10 shows representative H&E stained tissues slices of quadriceps muscle in tumor-bearing mice.

FIG. 11 shows representative data illustrating the frequency analysis of diameters of myofibers from tumor-bearing mice.

FIG. 12 shows a representative crystal structure illustrating that compounds B1 and C3 modulate interactions between subunits within the a ring and connections between the α and β subunits (10 Å proximity, top view shown).

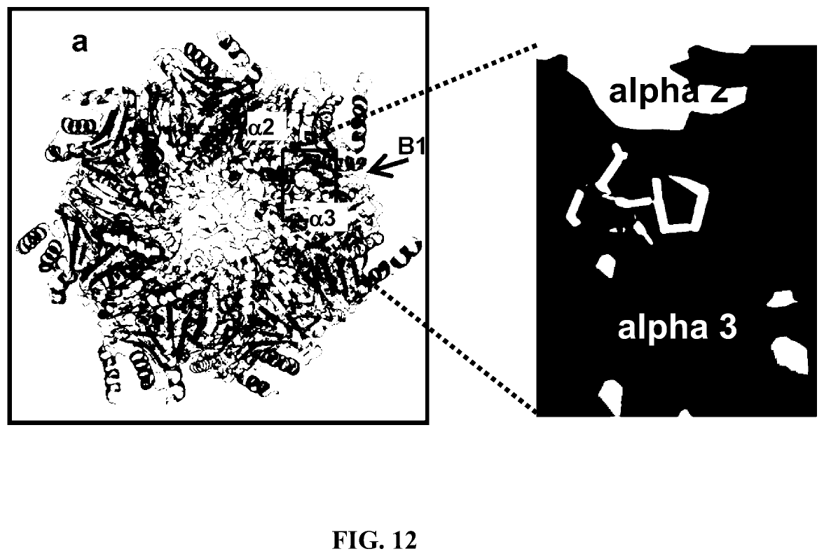

FIG. 13A and FIG. 13B show representative crystal structures illustrating an overlay of top poses of compound nos. B1 and C3. 10 Å proximity, top (FIG. 13A) and side (FIG. 13B) views with charge-colored surfaces are shown. The trimethoxyphenyl and benzyl carbamate groups are open for modifications.

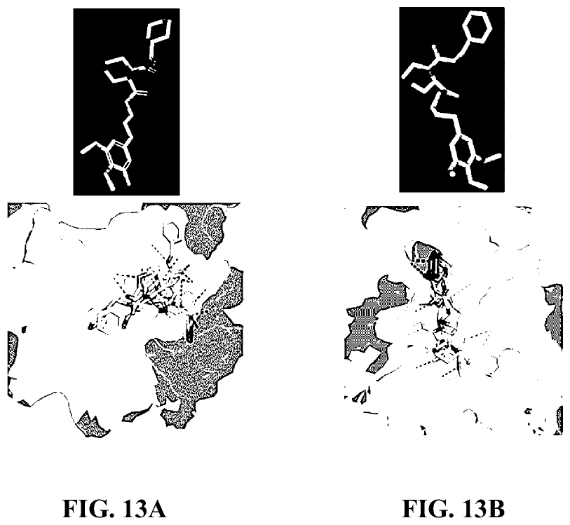

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the terms “muscle wasting” and “muscle atrophy” mean a weakening, shrinking, and/or loss of muscle caused by disease or lack of use that decreases strength and the ability to move. For example, muscle wasting can be due to a disease (e.g., cancer, diabetes, heart failure, chronic kidney disease, chronic obstructive pulmonary disease) or due to acute critical illnesses (e.g., burns, sepsis, trauma). Alternatively, muscle wasting can also be observed without any overt disease (e.g., sarcopenia).

As used herein, the terms “cachexia” and “muscle cachexia” refers to a type of muscle wasting due to a complex metabolic syndrome associated with underlying illness such as, for example, cancer, AIDS, and other chronic diseases. Cachexia can be further characterized by a debilitating loss in muscle mass and function, which ultimately deteriorates the patients' quality of life and dampens therapeutic treatment efficacy.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage form can comprise a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14 edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. For example, the cycloalkyl group and heterocycloalkyl group can be substituted with 0, 1, 2, 3, or 4 groups independently selected from C1-C4 alkyl, C3-C7 cycloalkyl, C1-C4 alkoxy, —NH₂, (C1-C4) alkylamino, (C1-C4)(C1-C4) dialkylamino, ether, halogen, —OH, C1-C4 hydroxyalkyl, —NO₂, silyl, sulfo-oxo, —SH, and C1-C4 thioalkyl, as described herein.

The term “polyalkylene group” as used herein is a group having two or more CH₂ groups linked to one another. The polyalkylene group can be represented by the formula (CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. For example, the cycloalkenyl group and heterocycloalkenyl group can be substituted with 0, 1, 2, 3, or 4 groups independently selected from C1-C4 alkyl, C3-C7 cycloalkyl, C1-C4 alkoxy, C2-C4 alkenyl, C3-C6 cycloalkenyl, C2-C4 alkynyl, aryl, heteroaryl, aldeyhyde, —NH₂, (C1-C4) alkylamino, (C1-C4)(C1-C4) dialkylamino, carboxylic acid, ester, ether, halogen, —OH, C1-C4 hydroxyalkyl, ketone, azide, —NO₂, silyl, sulfo-oxo, —SH, and C1-C4 thioalkyl, as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula C(O)H. Throughout this specification “C(O)” or “CO” is a short hand notation for a carbonyl group, i.e., C═O.

The terms “amine” or “amino” as used herein are represented by the formula NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxy” or “hydroxyl” as used herein is represented by the formula OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN or —C≡N.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C1-4 straight or branched alkylene)C(O)O—N(R^{o})₂, wherein each R^{o} may be substituted as defined below and is independently hydrogen, C₁-6 aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)O₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, -SiR^{•}₃, —OSiR^{•}₃, —C(O)SR^{•}, —(C1-4 straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)o-Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR^{•}₂, ═NNHC(O)R^{•}, ═NNHC(O)OR^{•}, ═NNHS(O)₂R^{•}, ═NR^{•}, ═NOR^{•}, —O(C(R^{•}₂))₂₋₃O—, or —S(C(R^{•}₂))₂₋₃S—, wherein each independent occurrence of R^{•} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{•}₂)₂₋₃O—, wherein each independent occurrence of R^{•} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{•} include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

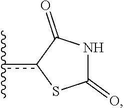

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an a-hydrogen can exist in an equilibrium of the keto form and the enol form.

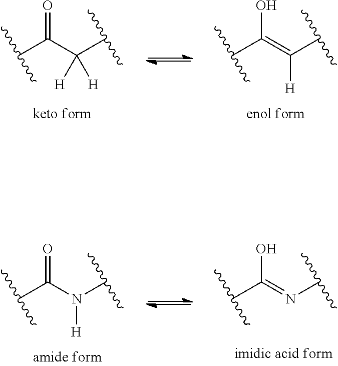 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

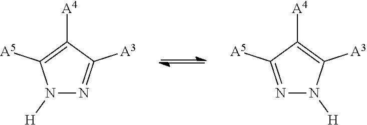

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

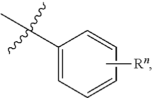

which is understood to be equivalent to a formula:

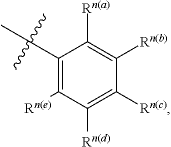

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, disclosed are compounds useful in treating or preventing muscle wasting such as, for example, muscle wasting due to human immunodeficiency virus (HIV), chronic renal failure, kidney disease, chronic obstructive pulmonary disease (COPD), multiple sclerosis, cystic fibrosis, rheumatoid arthritis, advanced dementia, cachexia, congestive heart failure (CHF), cancer, a limb injury, or immobilization. The disclosed compounds can be prepared by methods known to one of skill in the art. See, e.g., U.S. Pat. Nos. 9,918,968, 10,167,259, 11,020,383, and 11,345,659.

In one aspect, the compounds of the invention are useful in the treatment or prevention of disorders associated with the proteasome, as further described herein. In a further aspect, the disclosed compounds exhibit allosteric regulation of the proteasome.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, the compound has a structure represented by a formula selected from:

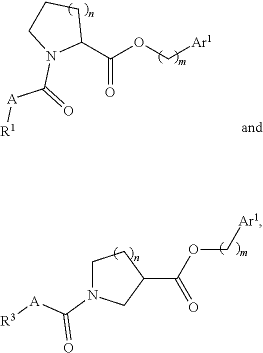

wherein n is 1 or 2; wherein m is 1, 2, or 3; wherein A is selected from —O— and —C(O)—; wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl); wherein q is 1, 2, or 3; wherein each of R^{20a} and R^{20b}, when present, is independently selected from hydrogen and C1-C4 alkyl; wherein Cy¹ is selected from cyclohexyl, a 6-membered heterocycloalkyl, and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; wherein R³ is selected from C1-C8 alkyl, (CH₂)_{q}Cy¹, and Cy¹; and wherein Ar¹ is selected from C6-aryl and pyridinyl, and is substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof.

In various aspects, the compound has a structure represented by a formula selected from:

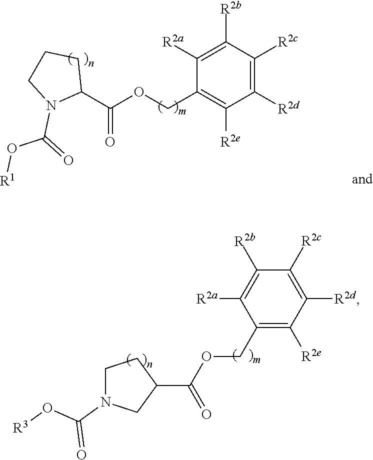

wherein n is 1 or 2; wherein m is 1, 2, or 3; wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl); wherein q, when present, is 1, 2, or 3; wherein each of R^{20a} and R^{20b}, when present, is independently selected from hydrogen and C1-C4 alkyl; wherein Cy¹ is selected from cyclohexyl and 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; and wherein each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; and wherein R³, when present, is selected from C1-C8 alkyl, (CH₂)_{q}Cy¹, and Cy¹, or a pharmaceutically acceptable salt thereof. In a further aspect, at least one of R^{2a}, R^{2b}, R^{2c}, R^{2d}, and R^{2e} is —OH, C1-C4 alkoxy, or C1-C4 haloalkoxy. In a still further aspect, when at least one of R^{2a}, R^{2b}, R^{2c}, R^{2d}, and R^{2e} is C1-C4 alkoxy and R¹ is C1-C8 acyclic alkyl, then R¹ is substituted with 1, 2, or 3 groups independently selected from halogen, —CN, —NH₂, —NO₂, —OH, —C(O)NR^{20a}R^{20b}, —CO₂H, and —CO₂(C1-C4 alkyl). In yet a further aspect, at least one of R^{2a}, R^{2b}, R^{2c}, R^{2d}, and R^{2e} is C1-C4 alkoxy or C1-C4 haloalkoxy. In an even further aspect, each of R^{2b}, R^{2c}, and R^{2d} is methoxy.

In various aspects, the compound has a structure represented by a formula selected from:

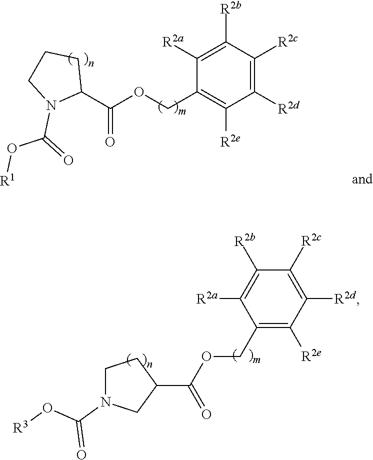

wherein n is 1 or 2; wherein m is 1, 2, or 3; wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl); wherein q, when present, is 1, 2, or 3; wherein each of R^{20a} and R^{20b}, when present, is independently selected from hydrogen and C1-C4 alkyl; wherein Cy¹ is selected from cyclohexyl and 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; and wherein each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; and wherein R³, when present, is selected from C1-C8 alkyl, (CH₂)_{q}Cy¹, and Cy¹, provided at least one of R^{2a}, R^{2b}, R^{2c}, R^{2d}, and R^{2e} is —OH, C1-C4 alkoxy, or C1-C4 haloalkoxy, and provided when at least one of R^{2a}, R^{2b}, R^{2c}, R^{2d}, and R^{2e} is C1-C4 alkoxy and R¹ is C1-C8 acyclic alkyl, then R¹ is substituted with 1, 2, or 3 groups independently selected from halogen, —CN, —NH₂, —NO₂, —OH, —C(O)NR^{20a}R^{20b}, —CO₂H, and —CO₂(C1-C4 alkyl), or a pharmaceutically acceptable salt thereof.

In various aspects, the compound has a structure selected from:

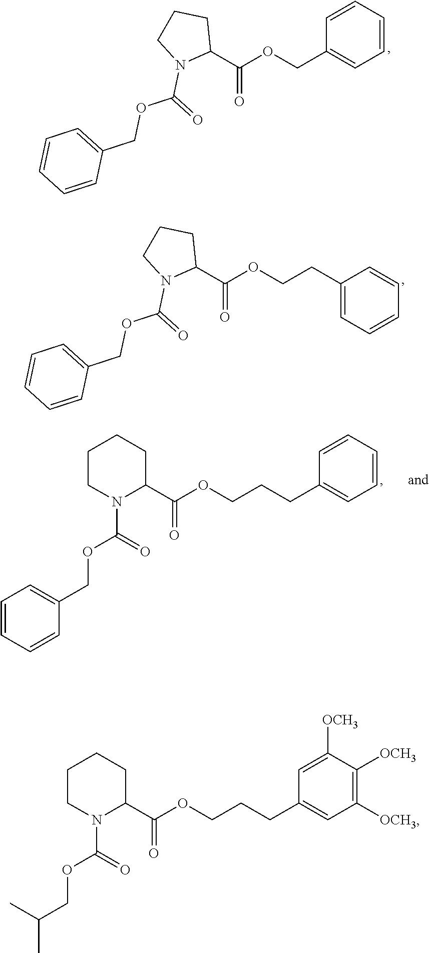 [SMILES: O=C(OCc1ccccc1)C1CCCN1C(=O)OCc1ccccc1 ; O=C(OCCc1ccccc1)C1CCCN1C(=O)OCc1ccccc1 ; O=C(OCCCc1ccccc1)C1CCCCN1C(=O)OCc1ccccc1 ; COc1cc(CCCOC(=O)C2CCCCN2C(=O)OCC(C)C)cc(OC)c1OC]

or a pharmaceutically acceptable salt thereof.

In a further aspect, the compound has a structure represented by a formula selected from:

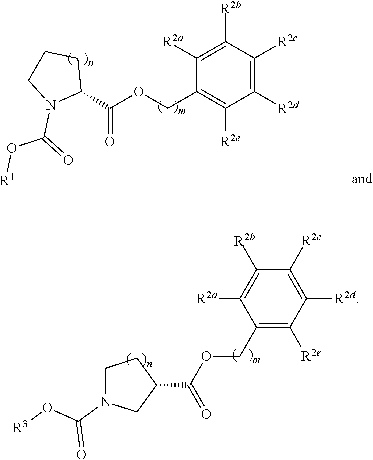

In a further aspect, the compound has a structure represented by a formula selected from:

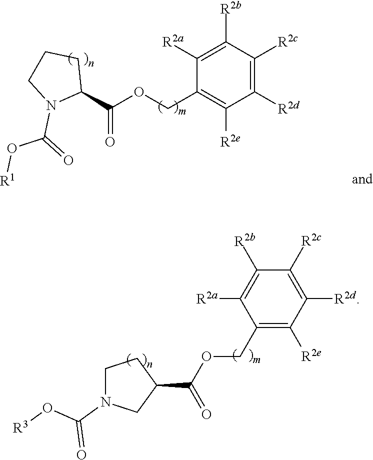

In a further aspect, the compound has a structure represented by a formula:

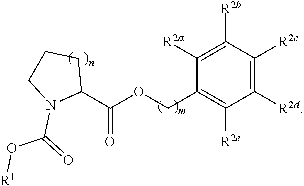

In a further aspect, the compound has a structure represented by a formula:

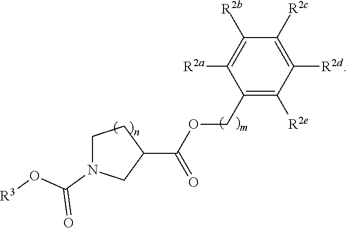

In a further aspect, the compound has a structure represented by a formula selected from:

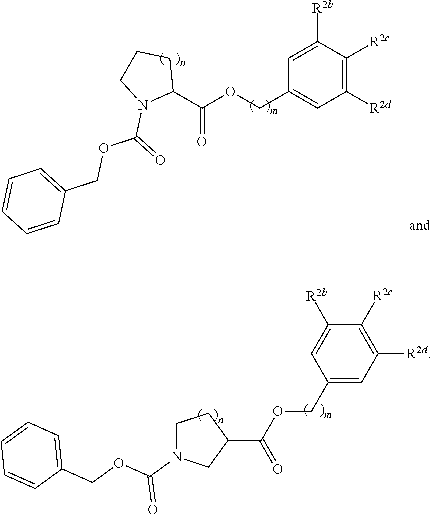

In a further aspect, the compound has a structure represented by a formula selected from:

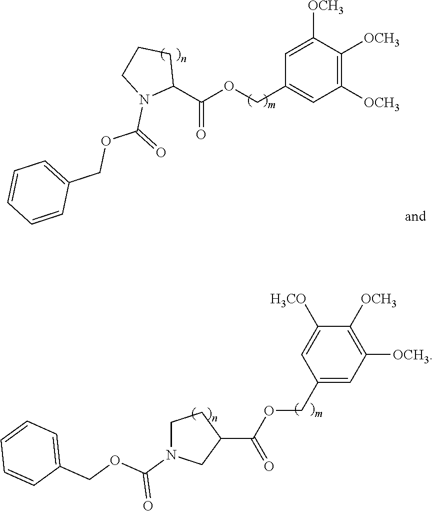

In a further aspect, the compound has a structure represented by a formula selected from:

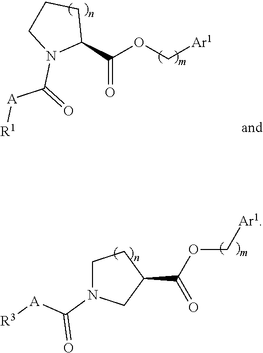

In a further aspect, the compound has a structure represented by a formula:

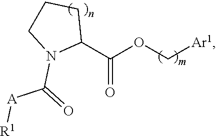

or a pharmaceutically acceptable salt thereof.

In a further aspect, the compound has a structure represented by a formula:

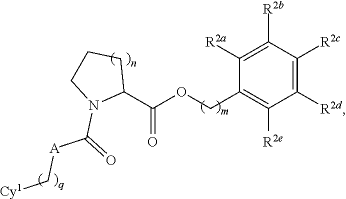

wherein each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof.

In a further aspect, the compound has a structure represented by a formula:

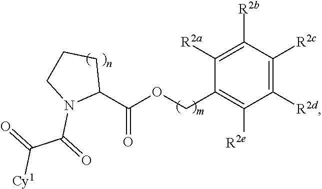

or a pharmaceutically acceptable salt thereof.

In a further aspect, the compound has a structure selected from:

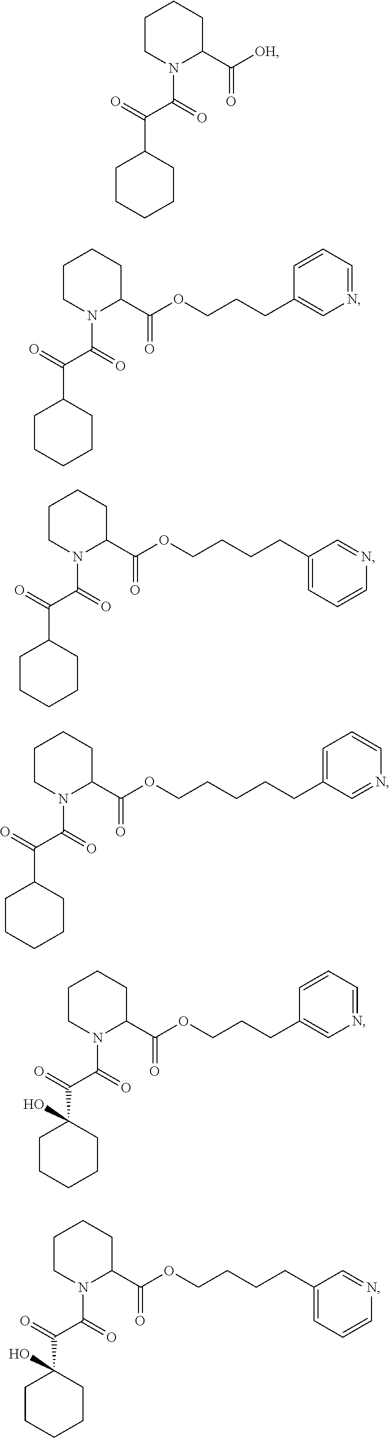

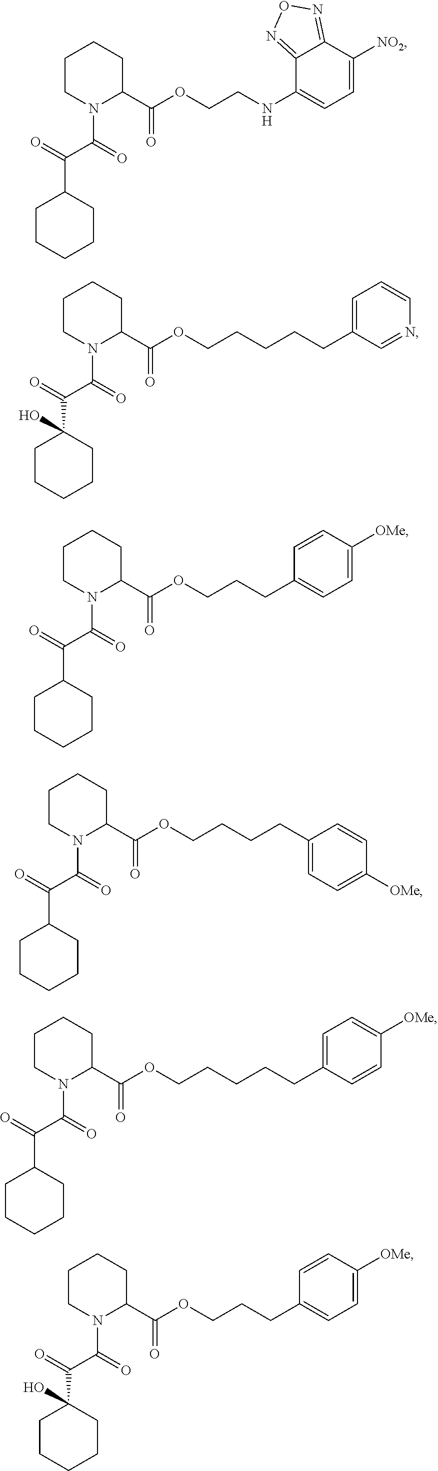

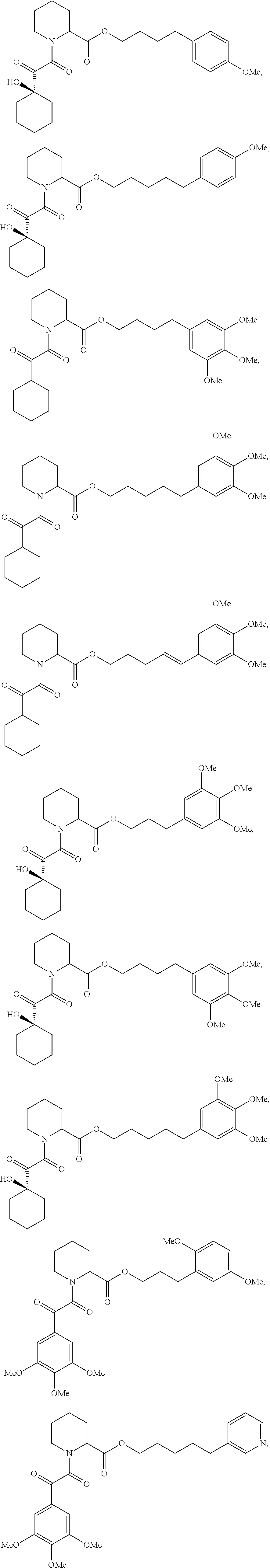

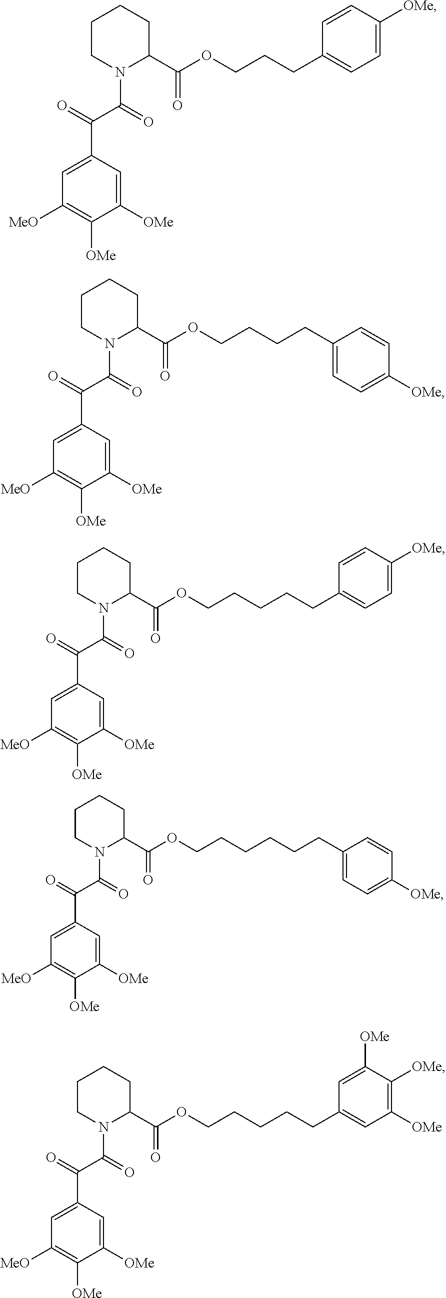

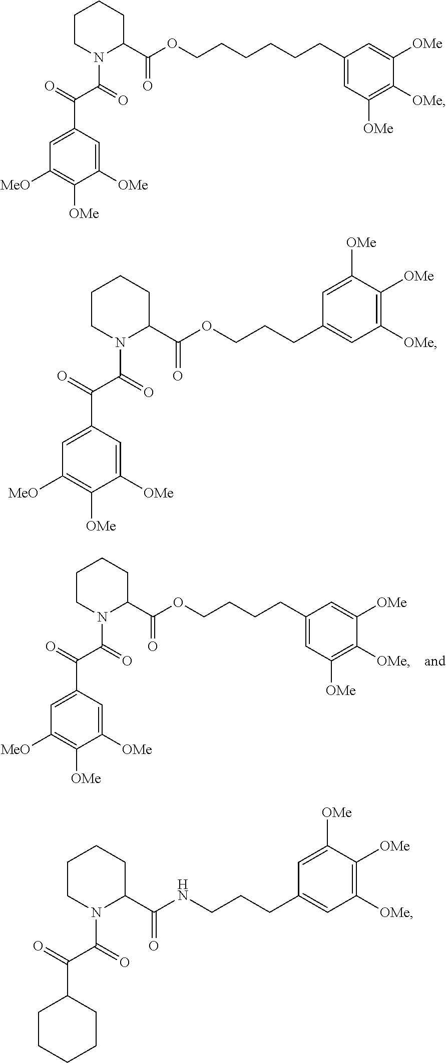

or a pharmaceutically acceptable salt thereof.

In a further aspect, the compound has a structure selected from:

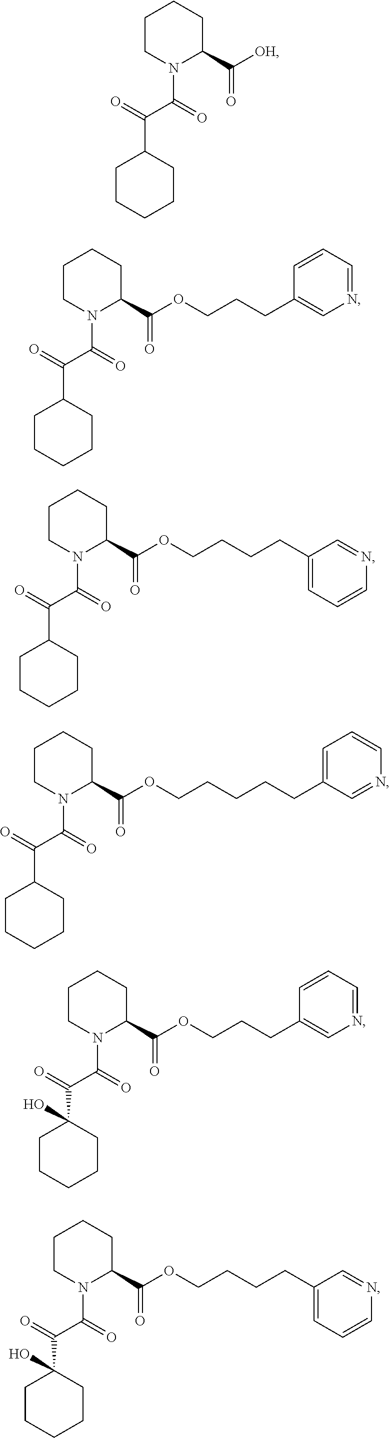

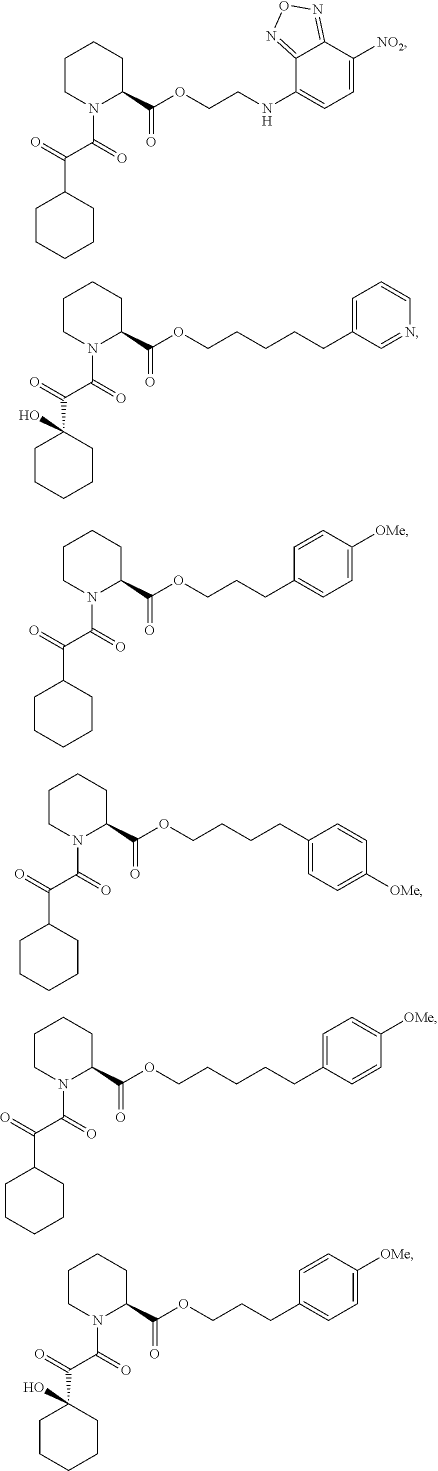

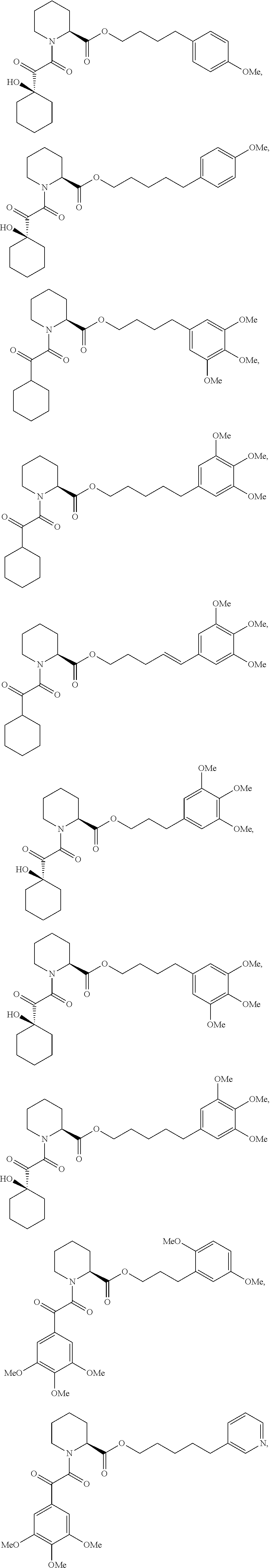

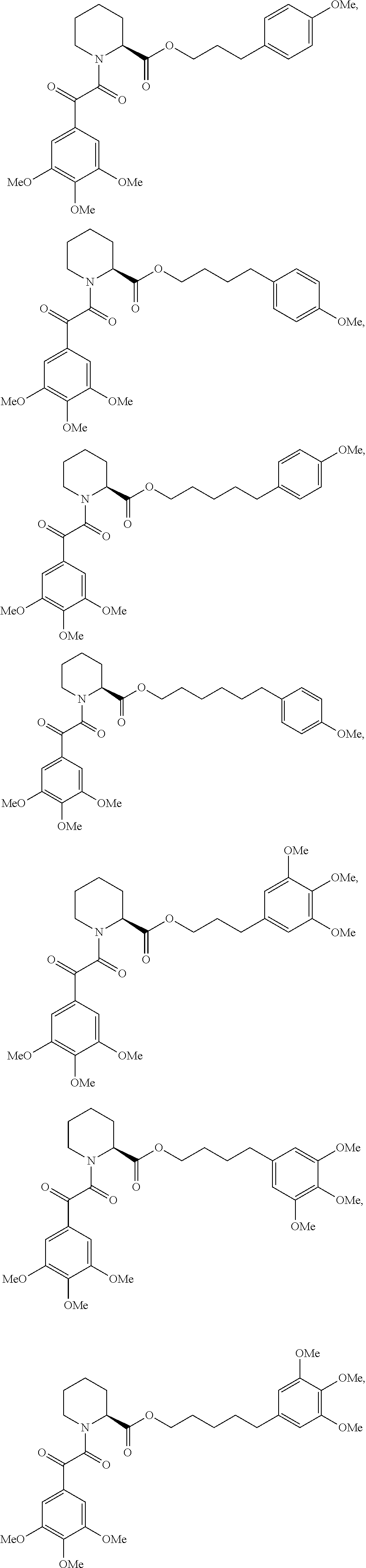

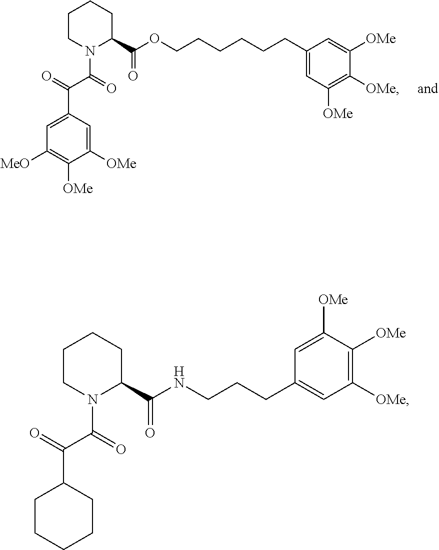 [SMILES: COc1cc(CCCCCCOC(=O)[C@@H]2CCCCN2C(=O)C(=O)c2cc(OC)c(OC)c(OC)c2)cc(OC)c1OC ; COc1cc(CCCNC(=O)[C@@H]2CCCCN2C(=O)C(=O)C2CCCCC2)cc(OC)c1OC]

or a pharmaceutically acceptable salt thereof.

In a further aspect, the compound has a structure:

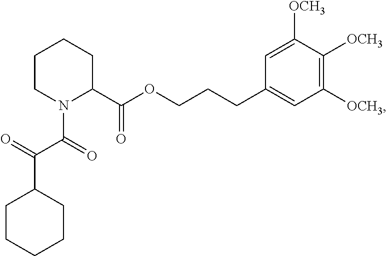 [SMILES: COc1cc(CCCOC(=O)C2CCCCN2C(=O)C(=O)C2CCCCC2)cc(OC)c1OC]

or a pharmaceutically acceptable salt thereof.

In a further aspect, the compound has a structure:

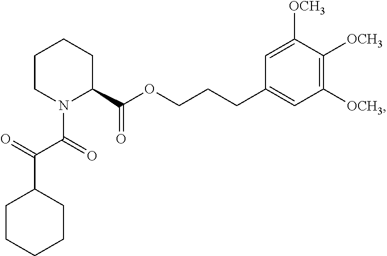 [SMILES: COc1cc(CCCOC(=O)[C@@H]2CCCCN2C(=O)C(=O)C2CCCCC2)cc(OC)c1OC]

or a pharmaceutically acceptable salt thereof.

In a further aspect, the compound has a structure represented by a formula:

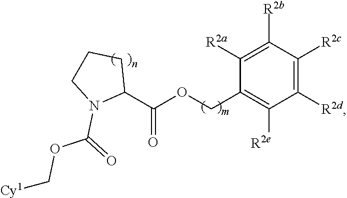

or a pharmaceutically acceptable salt thereof.

In a further aspect, the compound has a structure selected from:

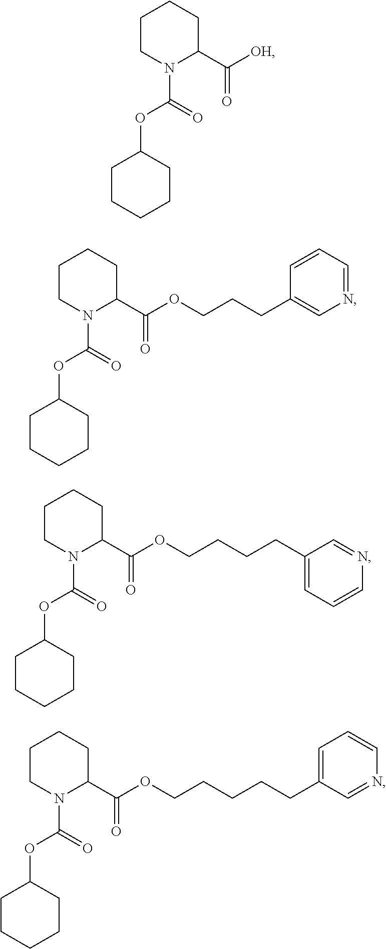

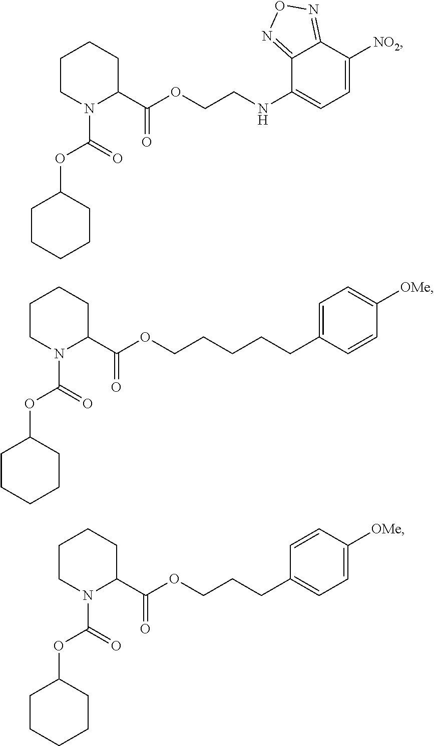

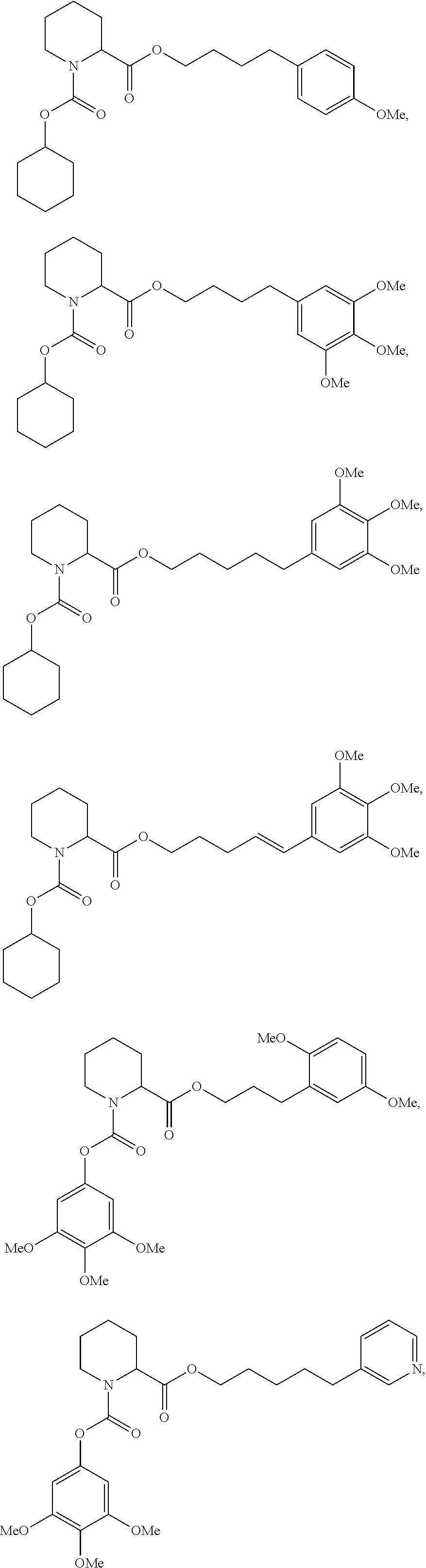

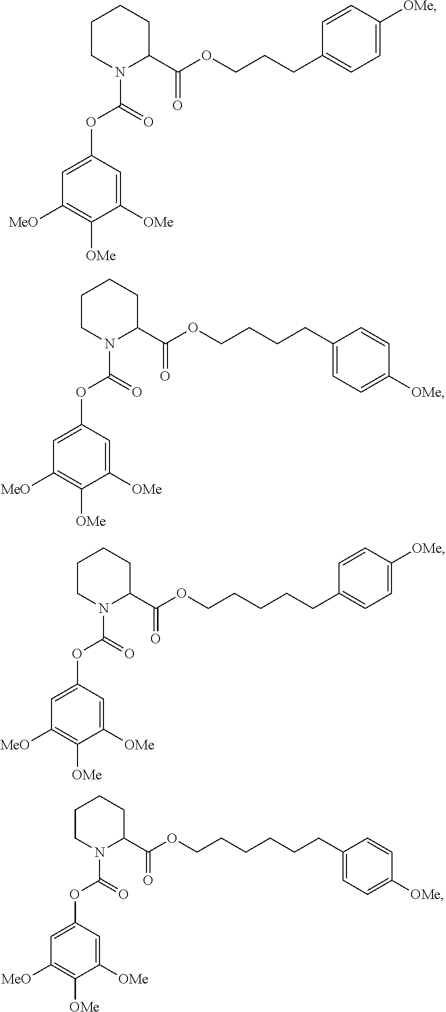

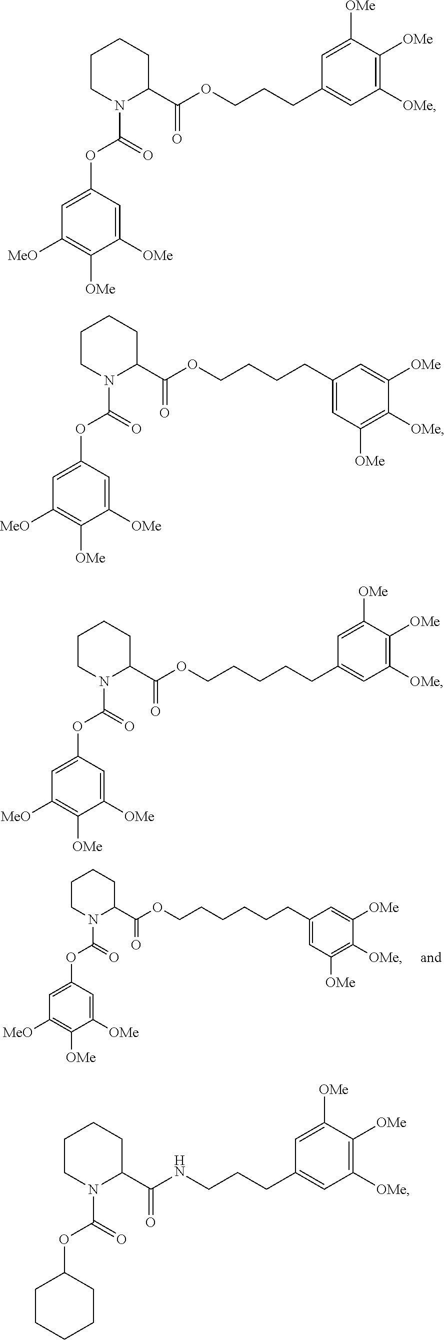

or a pharmaceutically acceptable salt thereof.

In a further aspect, the compound has a structure selected from:

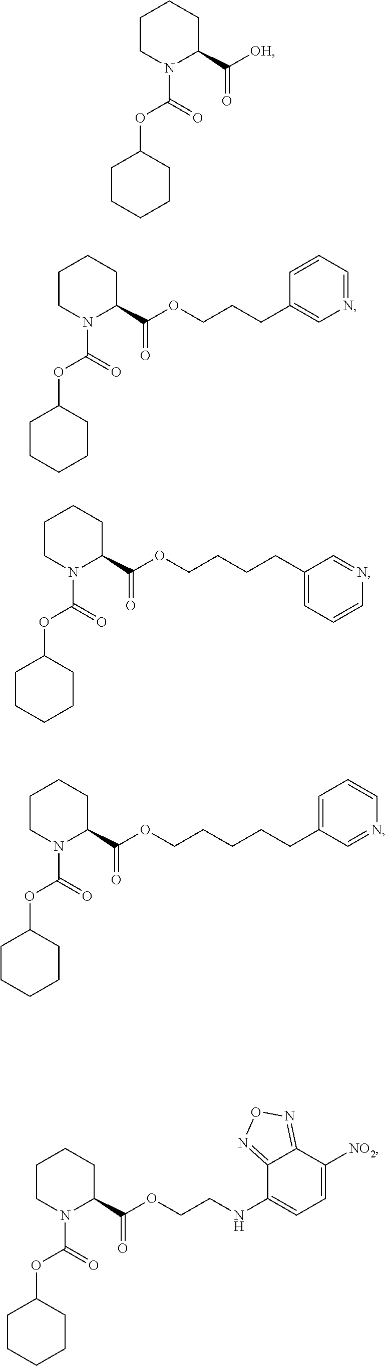

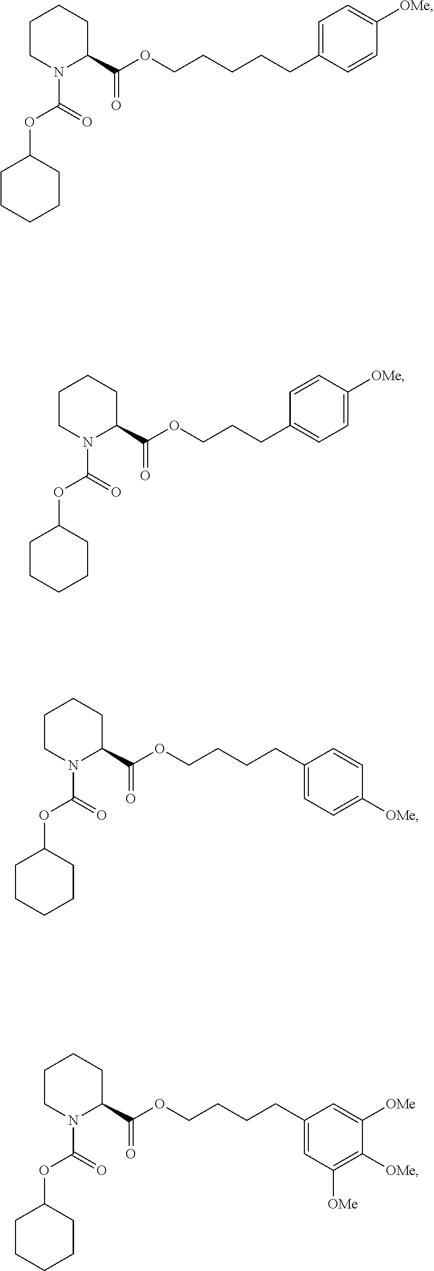 [SMILES: COc1ccc(CCCCCOC(=O)[C@@H]2CCCCN2C(=O)OC2CCCCC2)cc1 ; COc1ccc(CCCOC(=O)[C@@H]2CCCCN2C(=O)OC2CCCCC2)cc1 ; COc1ccc(CCCCOC(=O)[C@@H]2CCCCN2C(=O)OC2CCCCC2)cc1 ; COc1cc(CCCCOC(=O)[C@@H]2CCCCN2C(=O)OC2CCCCC2)cc(OC)c1OC]

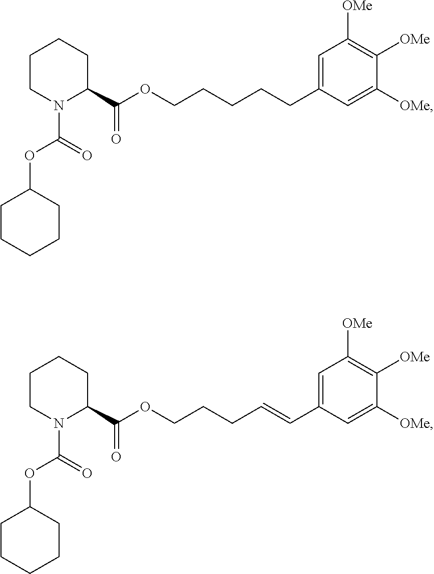 [SMILES: COc1cc(CCCCCOC(=O)[C@@H]2CCCCN2C(=O)OC2CCCCC2)cc(OC)c1OC ; COc1cc(/C=C/CCCOC(=O)[C@@H]2CCCCN2C(=O)OC2CCCCC2)cc(OC)c1OC]

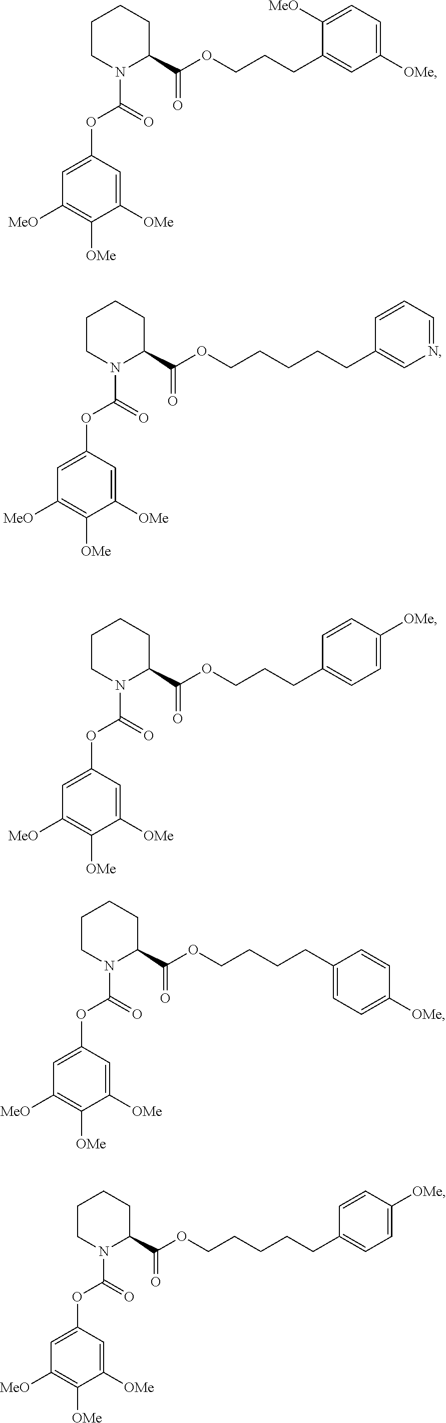

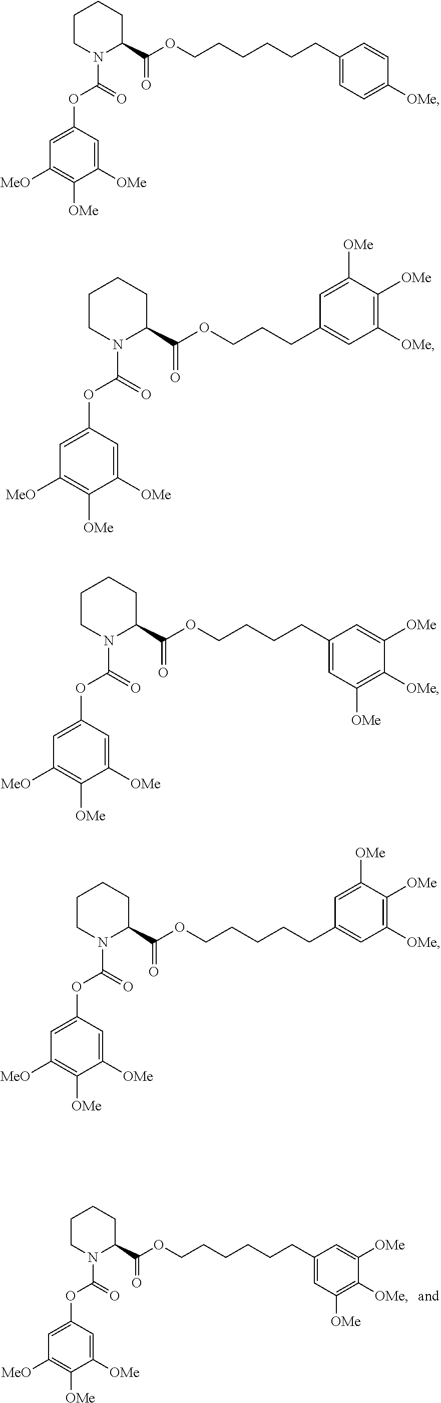 [SMILES: COc1ccc(CCCCCCOC(=O)[C@@H]2CCCCN2C(=O)Oc2cc(OC)c(OC)c(OC)c2)cc1 ; COc1cc(CCCOC(=O)[C@@H]2CCCCN2C(=O)Oc2cc(OC)c(OC)c(OC)c2)cc(OC)c1OC ; COc1cc(CCCCOC(=O)[C@@H]2CCCCN2C(=O)Oc2cc(OC)c(OC)c(OC)c2)cc(OC)c1OC ; COc1cc(CCCCCOC(=O)[C@@H]2CCCCN2C(=O)Oc2cc(OC)c(OC)c(OC)c2)cc(OC)c1OC ; COc1cc(CCCCCCOC(=O)[C@@H]2CCCCN2C(=O)Oc2cc(OC)c(OC)c(OC)c2)cc(OC)c1OC]

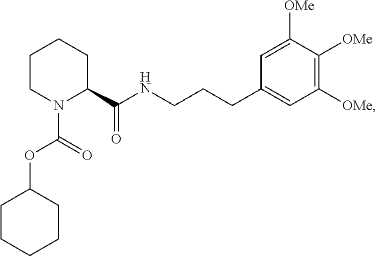 [SMILES: COc1cc(CCCNC(=O)[C@@H]2CCCCN2C(=O)OC2CCCCC2)cc(OC)c1OC]

or a pharmaceutically acceptable salt thereof.

In a further aspect, the compound is selected from:

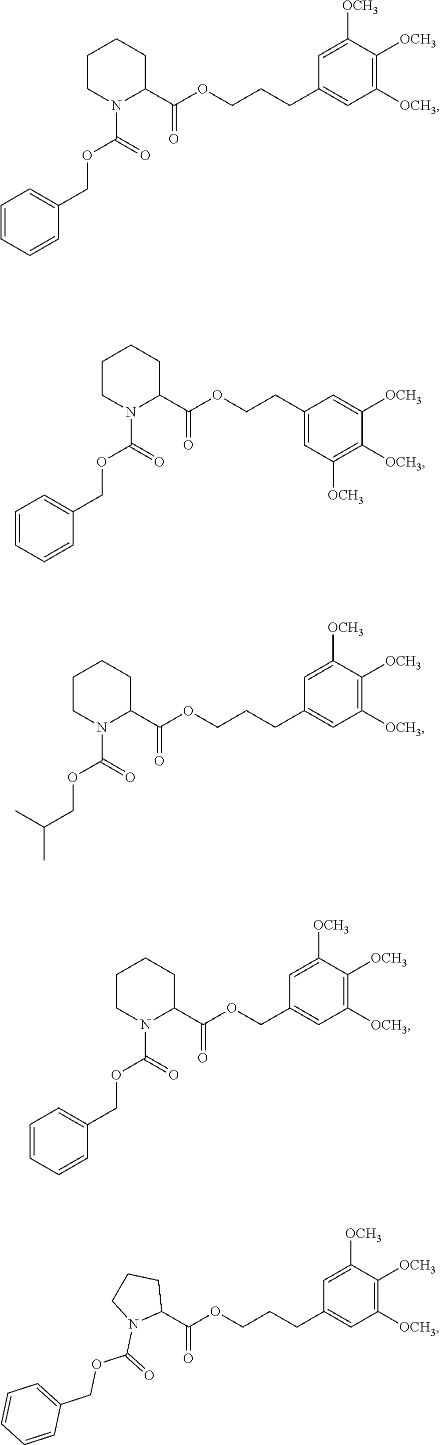 [SMILES: COc1cc(CCCOC(=O)C2CCCCN2C(=O)OCc2ccccc2)cc(OC)c1OC ; COc1cc(CCOC(=O)C2CCCCN2C(=O)OCc2ccccc2)cc(OC)c1OC ; COc1cc(CCCOC(=O)C2CCCCN2C(=O)OCC(C)C)cc(OC)c1OC ; COc1cc(COC(=O)C2CCCCN2C(=O)OCc2ccccc2)cc(OC)c1OC ; COc1cc(CCCOC(=O)C2CCCN2C(=O)OCc2ccccc2)cc(OC)c1OC]

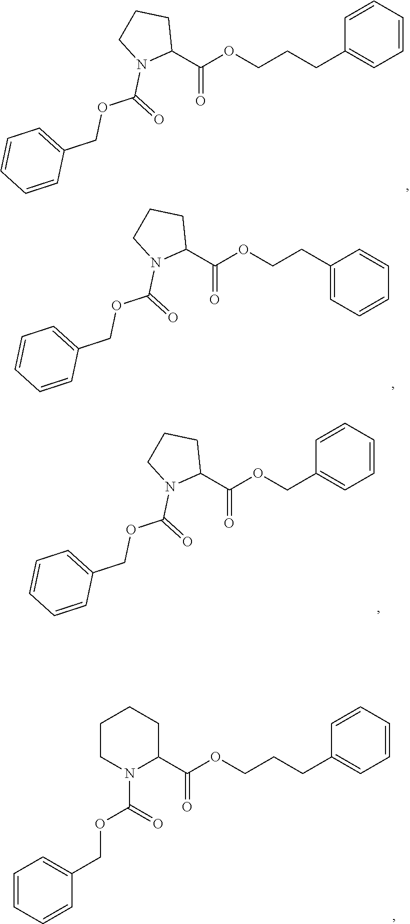 [SMILES: O=C(OCCCc1ccccc1)C1CCCN1C(=O)OCc1ccccc1 ; O=C(OCCc1ccccc1)C1CCCN1C(=O)OCc1ccccc1 ; O=C(OCc1ccccc1)C1CCCN1C(=O)OCc1ccccc1 ; O=C(OCCCc1ccccc1)C1CCCCN1C(=O)OCc1ccccc1]

or a pharmaceutically acceptable salt thereof.

In a further aspect, the compound is selected from:

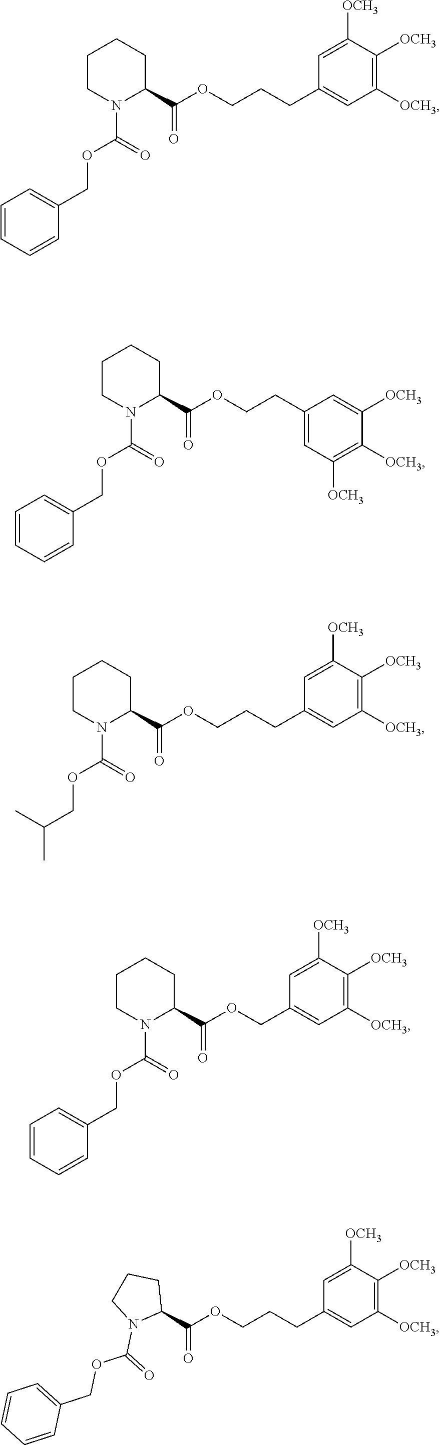 [SMILES: COc1cc(CCCOC(=O)[C@@H]2CCCCN2C(=O)OCc2ccccc2)cc(OC)c1OC ; COc1cc(CCOC(=O)[C@@H]2CCCCN2C(=O)OCc2ccccc2)cc(OC)c1OC ; COc1cc(CCCOC(=O)[C@@H]2CCCCN2C(=O)OCC(C)C)cc(OC)c1OC ; COc1cc(COC(=O)[C@@H]2CCCCN2C(=O)OCc2ccccc2)cc(OC)c1OC ; COc1cc(CCCOC(=O)[C@@H]2CCCN2C(=O)OCc2ccccc2)cc(OC)c1OC]

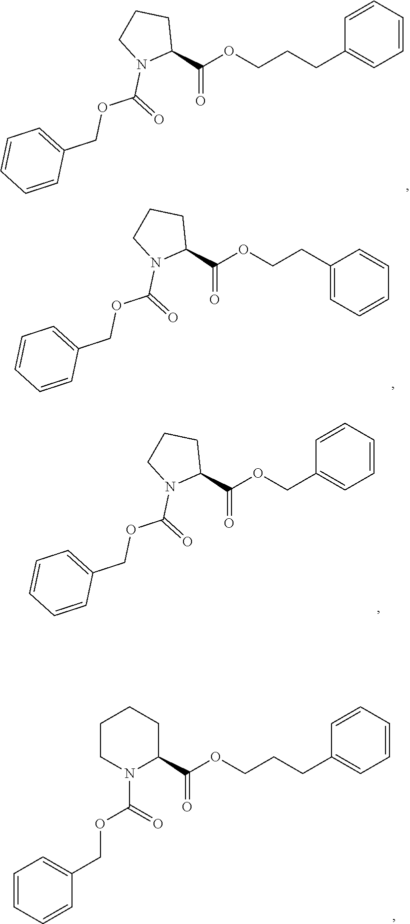 [SMILES: O=C(OCCCc1ccccc1)[C@@H]1CCCN1C(=O)OCc1ccccc1 ; O=C(OCCc1ccccc1)[C@@H]1CCCN1C(=O)OCc1ccccc1 ; O=C(OCc1ccccc1)[C@@H]1CCCN1C(=O)OCc1ccccc1 ; O=C(OCCCc1ccccc1)[C@@H]1CCCCN1C(=O)OCc1ccccc1]

or a pharmaceutically acceptable salt thereof.

In a further aspect, the compound is:

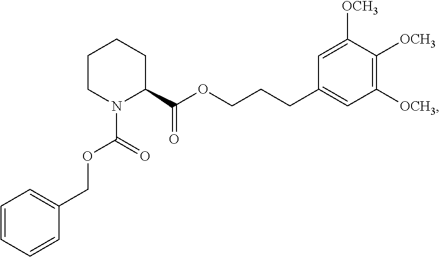 [SMILES: COc1cc(CCCOC(=O)[C@@H]2CCCCN2C(=O)OCc2ccccc2)cc(OC)c1OC]

or a pharmaceutically acceptable salt thereof.

In a further aspect, the compound has a structure:

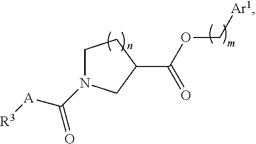

or a pharmaceutically acceptable salt thereof.

In a further aspect, the compound has a structure represented by a formula:

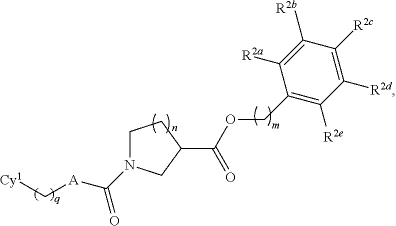

wherein each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof.

In a further aspect, the compound has a structure represented by a formula:

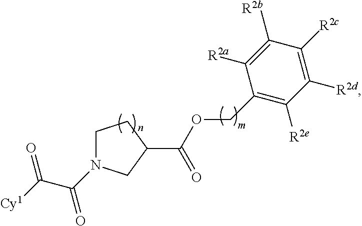

or a pharmaceutically acceptable salt thereof.

In a further aspect, the compound has a structure selected from:

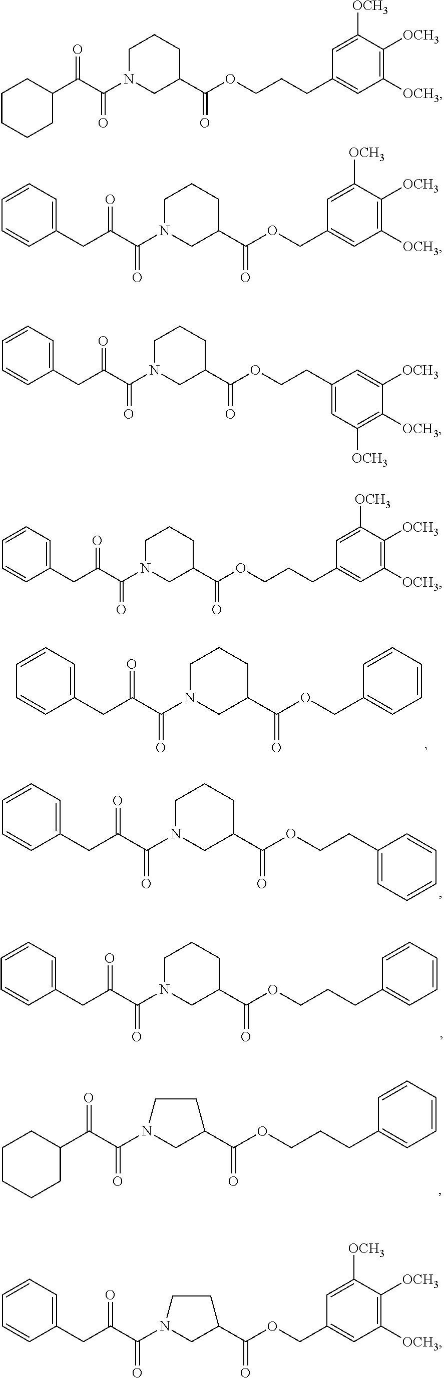

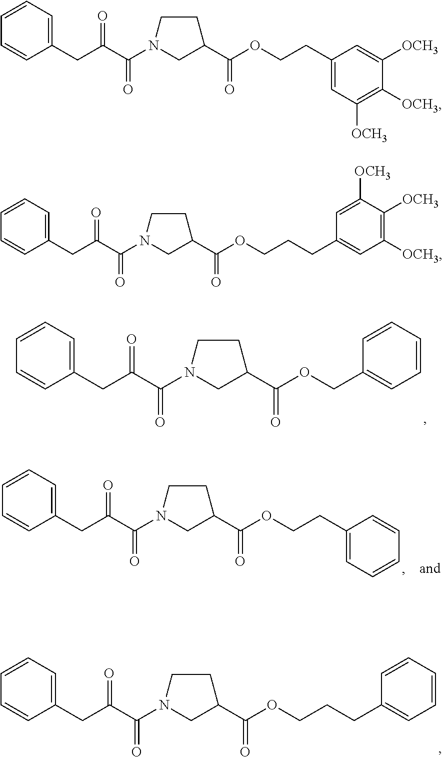

or a pharmaceutically acceptable salt thereof.

In a further aspect, the compound has a structure selected from:

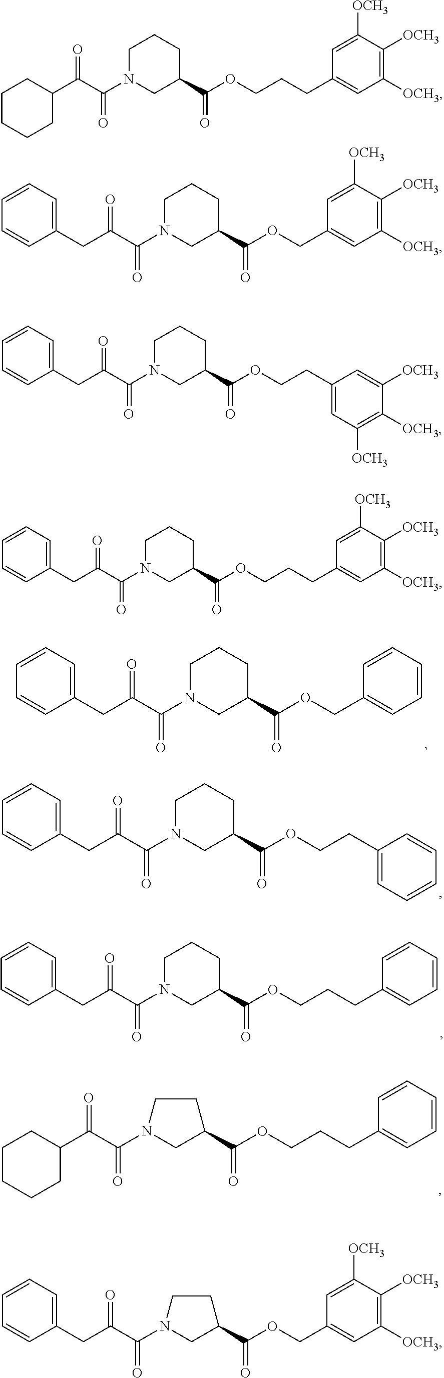

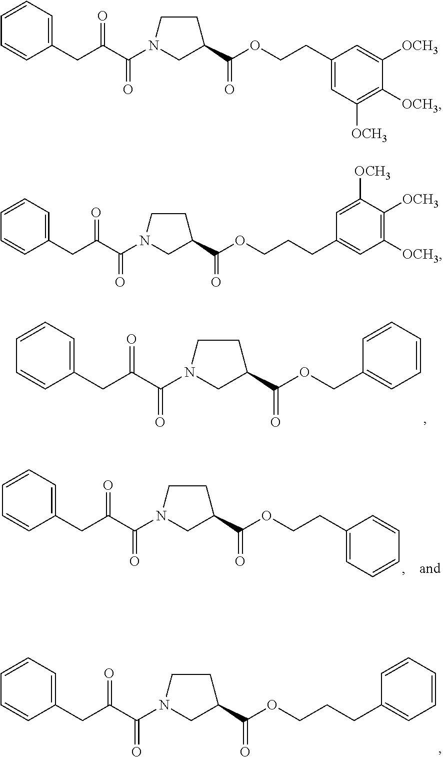

or a pharmaceutically acceptable salt thereof.

In a further aspect, the compound has a structure represented by a formula:

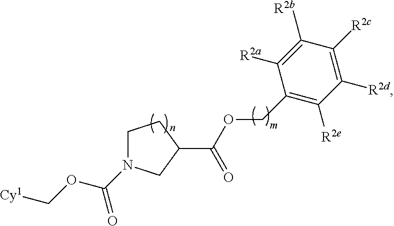

or a pharmaceutically acceptable salt thereof.

In a further aspect, the compound has a structure selected from:

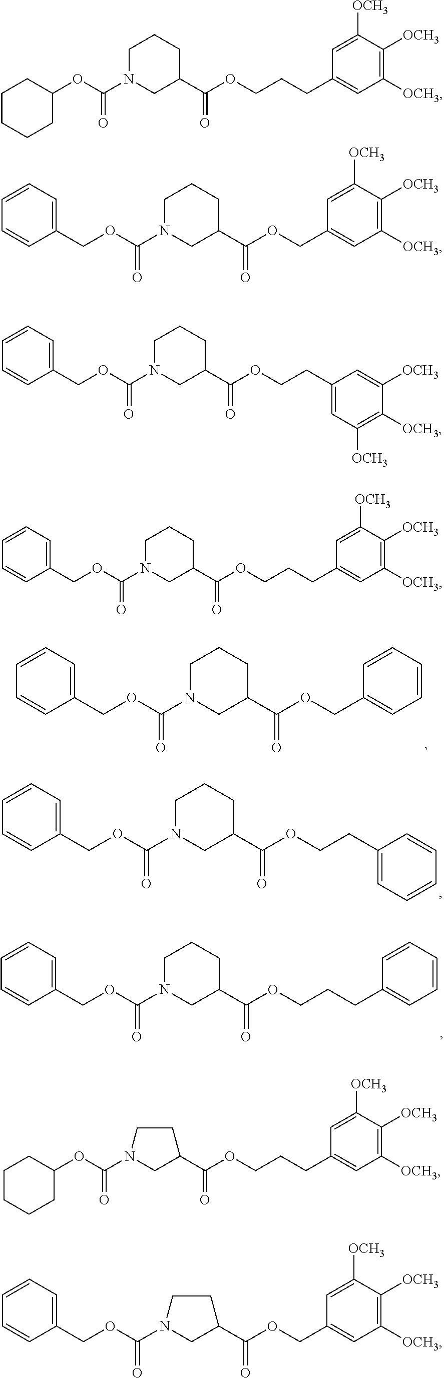

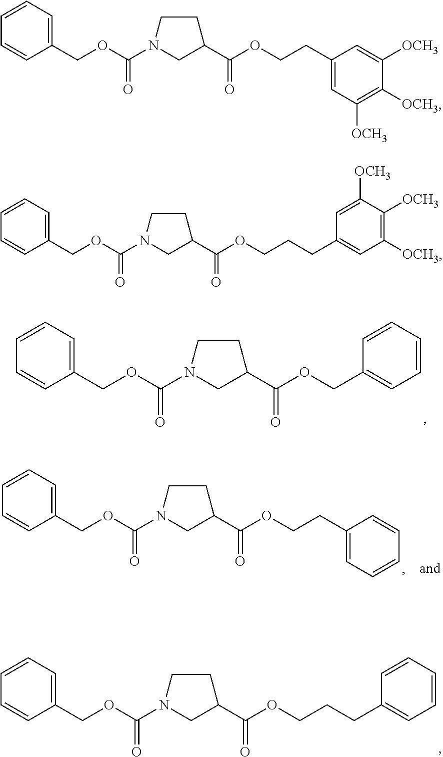

or a pharmaceutically acceptable salt thereof.

In a further aspect, the compound has a structure selected from:

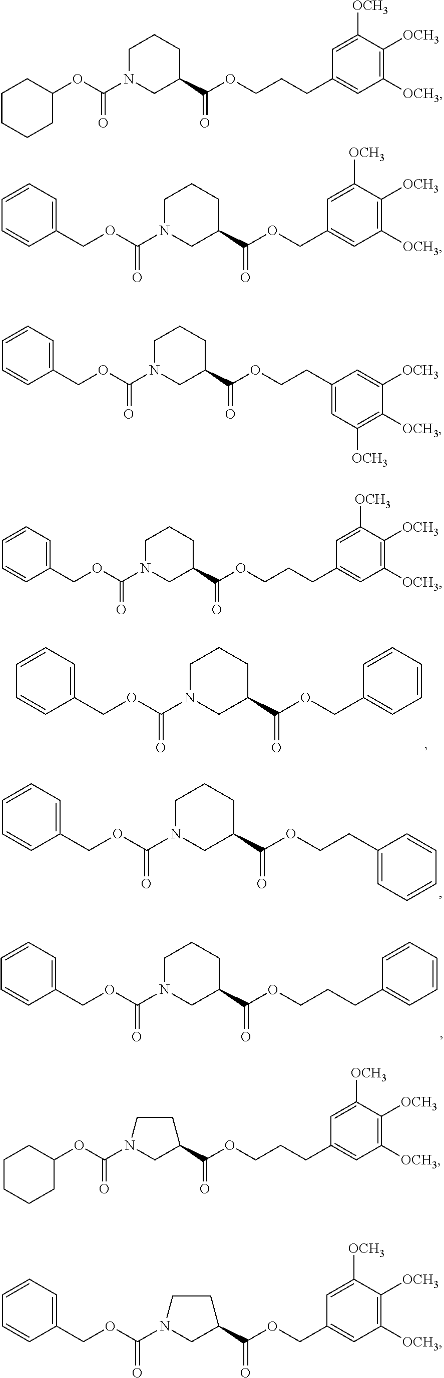

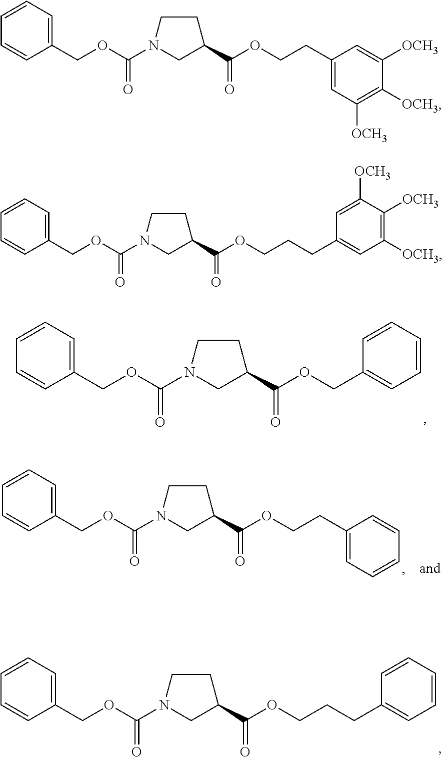

or a pharmaceutically acceptable salt thereof.

In a further aspect, the compound is:

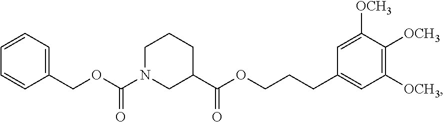 [SMILES: COc1cc(CCCOC(=O)C2CCCN(C(=O)OCc3ccccc3)C2)cc(OC)c1OC]

or a pharmaceutically acceptable salt thereof.

In a further aspect, the compound is:

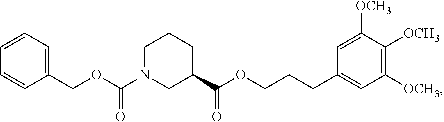 [SMILES: COc1cc(CCCOC(=O)[C@@H]2CCCN(C(=O)OCc3ccccc3)C2)cc(OC)c1OC]

or a pharmaceutically acceptable salt thereof.

In one aspect, n is 1 or 2. In a further aspect, n is 1. In a still further aspect, n is 2.

In one aspect, m is 1, 2, or 3. In a further aspect, m is 1 or 2. In a still further aspect, m is 2 or 3. In yet a further aspect, m is 1 or 3. In an even further aspect, m is 1. In a still further aspect, m is 2. In yet a further aspect, m is 3.

In one aspect, q, when present, is 1, 2, or 3. In a further aspect, q, when present, is 1 or 2. In a still further aspect, q, when present, is 2 or 3. In yet a further aspect, q, when present, is 1 or 3. In an even further aspect, q, when present, is 1. In a still further aspect, q, when present, is 2. In yet a further aspect, q, when present, is 3.

a. A Groups

In one aspect, A is selected from —O— and —C(O)—. In a further aspect, A is —O—. In a still further aspect, A is —C(O)—.

b. R¹ Groups

In one aspect, R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl). In a further aspect, R¹ is selected from (CH₂)_{q}Cy¹, Cy¹, and C2-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl).

In a further aspect, R¹ is C1-C8 acyclic alkyl substituted with 0, 1, 2, or 3 groups independently selected from halogen, —CN, —NH₂, —NO₂, —OH, —C(O)NR^{20a}R^{20b}, CO₂H, and —CO₂(C1-C4 alkyl). In a still further aspect, R¹ is C1-C8 acyclic alkyl substituted with 1, 2, or 3 groups independently selected from halogen, —CN, —NH₂, —NO₂, —OH, —C(O)NR^{20a}R^{20b}, —CO₂H, and —CO₂(C1-C4 alkyl). In yet a further aspect, R¹ is C1-C8 acyclic alkyl substituted with 0, 1, or 2 groups independently selected from halogen, —CN, —NH₂, —NO₂, —OH, —C(O)NR^{20a}R^{20b}, —CO₂H, and —CO₂(C1-C4 alkyl). In an even further aspect, R¹ is C1-C8 acyclic alkyl substituted with 0 or 1 group selected from halogen, —CN, —NH₂, —NO₂, —OH, —C(O)NR^{20a}R^{20b}, —CO₂H, and —CO₂(C1-C4 alkyl). In a still further aspect, R¹ is C1-C8 acyclic alkyl monosubstituted with a group selected from halogen, —CN, —NH₂, —NO₂, —OH, —C(O)NR^{20a}R^{20b}, —CO₂H, and —CO₂(C1-C4 alkyl). In yet a further aspect, R¹ is unsubstituted C1-C8 acyclic alkyl.

In a further aspect, R¹ is C1-C8 acyclic alkyl substituted with 1, 2, or 3 groups independently selected from halogen, —CN, —NH₂, —NO₂, —OH, —C(O)NR^{20a}R^{20b}, —CO₂H, and —CO₂(C1-C4 alkyl). In a still further aspect, R¹ is C1-C8 acyclic alkyl substituted with 1 or 2 groups independently selected from halogen, —CN, —NH₂, —NO₂, —OH, —C(O)NR^{20a}R^{20b}, —CO₂H, and —CO₂(C1-C4 alkyl). In yet a further aspect, R¹ is C1-C8 acyclic alkyl substituted with 2 or 3 groups independently selected from halogen, —CN, —NH₂, —NO₂, —OH, —C(O)NR^{20a}R^{20b}, —CO₂H, and —CO₂(C1-C4 alkyl). In an even further aspect, R¹ is C1-C8 acyclic alkyl substituted with 1 or 3 groups independently selected from halogen, —CN, —NH₂, —NO₂, —OH, —C(O)NR^{20a}R^{20b}, —CO₂H, and —CO₂(C1-C4 alkyl). In a still further aspect, R¹ is C1-C8 acyclic alkyl substituted with 2 groups independently selected from halogen, —CN, —NH₂, —NO₂, —OH, —C(O)NR^{20a}R^{20b}, —CO₂H, and —CO₂(C1-C4 alkyl). In yet a further aspect, R¹ is C1-C8 acyclic alkyl substituted with 3 groups independently selected from halogen, —CN, —NH₂, —NO₂, —OH, —C(O)NR^{20a}R^{20b}, —CO₂H, and —CO₂(C1-C4 alkyl).

In a further aspect, R¹ is C1-C8 acyclic alkyl substituted with 1, 2, or 3 groups independently selected from halogen, —CN, —NH₂, —NO₂, —OH, —C(O)NR^{20a}R^{20b}, —CO₂H, and —CO₂(C1-C4 alkyl). In a still further aspect, R¹ is selected from ethyl, n-propyl, isopropyl, n-butyl, isobutyl, sec-butyl, and t-butyl.

In a further aspect, R¹ is selected from (CH₂)_{q}Cy¹ and Cy¹. In a still further aspect, R¹ is (CH₂)_{q}Cy¹. In yet a further aspect, R¹ is Cy¹.

In a further aspect, R¹ is selected from CH₂Cy¹ and Cy¹. In a still further aspect, R¹ is CH₂Cy¹.

In a further aspect, R¹ is C1-C8 acyclic alkyl. In a still further aspect, R¹ is C1-C4 acyclic alkyl. In yet a further aspect, R¹ is selected from methyl, ethyl, n-propyl, and i-propyl. In an even further aspect, R¹ is selected from methyl and ethyl. In a still further aspect, R¹ is selected from n-propyl, and i-propyl. In yet a further aspect, R¹ is ethyl. In an even further aspect, R¹ is methyl.

c. R^{2A}, R^{2B}, R^{2C}, R^{2D}, and R^{2E} Groups

In one aspect, each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a further aspect, least one of R^{2a}, R^{2b}, R^{2c}, R^{2d}, and R^{2e} is —OH, C1-C4 alkoxy, or C1-C4 haloalkoxy. In a still further aspect, when at least one of R², R^{2b}, R^{2c}, R^{2d}, and R^{2e} is C1-C4 alkoxy and R¹ is C1-C8 acyclic alkyl, then R¹ is substituted with 1, 2, or 3 groups independently selected from halogen, —CN, —NH₂, —NO₂, —OH, —C(O)NR^{20a}R^{20b}, —CO₂H, and —CO₂(C1-C4 alkyl).

In a further aspect, each of R^{2a}, R^{2b}, R^{2c}, R^{2d}, and R^{2e} is hydrogen.

In a further aspect, each of R^{2a}, R^{2b}, R^{2c}, R^{2d}, and R^{2e} is independently selected from hydrogen, —F, —Cl, —CN, —NH₂, —NO₂, —OH, methyl, ethyl, n-propyl, i-propyl, n-butyl, t-butyl, s-butyl, i-butyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, and —N(CH₃)CH(CH₃)₂. In a still further aspect, each of R^{2a}, R^{2b}, R^{2c}, R^{2d}, and R^{2e} is independently selected from hydrogen, —F, —Cl, —CN, —NH₂, —NO₂, —OH, methyl, ethyl, n-propyl, i-propyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, and —N(CH₃)CH(CH₃)₂. In yet a further aspect, each of R^{2a}, R^{2b}, R^{2c}, R^{2d}, and R^{2e} is independently selected from hydrogen, —F, —Cl, —CN, —NH₂, —NO₂, —OH, methyl, ethyl, —CH₂F, —CH₂CH₂F, —CH₂Cl, —CH₂CH₂Cl, —CH₂OH, —CH₂CH₂OH, —OCH₃, —OCH₂CH₃, —NHCH₃, —NHCH₂CH₃, —N(CH₃)₂, —N(CH₃)CH₂CH₃, and —N(CH₂CH₃)₂. In an even further aspect, each of R^{2a}, R^{2b}, R^{2c}, R^{2d}, and R^{2e} is independently selected from hydrogen, —F, —Cl, —CN, —NH₂, —NO₂, —OH, methyl, —CH₂F, —CH₂Cl, —CH₂OH, —OCH₃, —NHCH₃, and —N(CH₃)₂.

In a further aspect, each of R^{2a}, R^{2b}, R^{2c}, R^{2d}, and R^{2e} is independently selected from hydrogen, —F, —Cl, —CN, —NH₂, —NO₂, —OH, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, and —N(CH₃)CH(CH₃)₂. In yet a further aspect, each of R^{2a}, R^{2b}, R^{2c}, R^{2d}, and R^{2e} is independently selected from hydrogen, —F, —Cl, —CN, —NH₂, —NO₂, —OH, —CH₂OH, —CH₂CH₂OH, —OCH₃, —OCH₂CH₃, —NHCH₃, —NHCH₂CH₃, —N(CH₃)₂, —N(CH₃)CH₂CH₃, and —N(CH₂CH₃)₂. In an even further aspect, each of R^{2a}, R^{2b}, R^{2c}, R^{2d}, and R^{2e} is independently selected from hydrogen, —F, —Cl, —CN, —NH₂, —NO₂, —OH, —CH₂OH, —OCH₃, —NHCH₃, and —N(CH₃)₂.

In a further aspect, each of R^{2a}, R^{2b}, R^{2c}, R^{2d}, and R^{2e} is independently selected from hydrogen, —F, —Cl, methyl, ethyl, n-propyl, i-propyl, n-butyl, t-butyl, s-butyl, i-butyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, and —N(CH₃)CH(CH₃)₂. In a still further aspect, each of R^{2a}, R^{2b}, R^{2c}, R^{2d}, and R^{2e} is independently selected from hydrogen, —F, —Cl, methyl, ethyl, n-propyl, i-propyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, and —N(CH₃)CH(CH₃)₂. In yet a further aspect, each of R^{2a}, R^{2b}, R^{2c}, R^{2d}, and R^{2e} is independently selected from hydrogen, —F, —Cl, methyl, ethyl, —CH₂F, —CH₂CH₂F, —CH₂Cl, —CH₂CH₂Cl, —CH₂OH, —CH₂CH₂OH, —OCH₃, —OCH₂CH₃, —NHCH₃, —NHCH₂CH₃, —N(CH₃)₂, —N(CH₃)CH₂CH₃, and —N(CH₂CH₃)₂. In an even further aspect, each of R^{2a}, R^{2b}, R^{2c}, R^{2d}, and R^{2e} is independently selected from hydrogen, —F, —Cl, methyl, —CH₂F, —CH₂Cl, —CH₂OH, —OCH₃, —NHCH₃, and —N(CH₃)₂.

In a further aspect, each of R^{2a}, R^{2b}, R^{2c}, R^{2d}, and R^{2e} is independently selected from hydrogen and halogen. In a still further aspect, each of R^{2a}, R^{2b}, R^{2c}, R^{2d}, and R^{2e} is independently selected from hydrogen, —F, —Cl, and —Br. In yet a further aspect, each of R^{2a}, R^{2b}, R^{2c}, R^{2d}, and R^{2e} is independently selected from hydrogen, —F, and —Cl. In an even further aspect, each of R^{2a}, R^{2b}, R^{2c}, R^{2d}, and R^{2e} is independently selected from hydrogen and —F. In a still further aspect, each of R^{2a}, R^{2b}, R^{2c}, R^{2d}, and R^{2e} is independently selected from hydrogen and —Cl.

In a further aspect, each of R^{2a}, R^{2b}, R^{2c}, R^{2d}, and R^{2e} is independently selected from hydrogen and C1-C4 alkyl. In a still further aspect, each of R^{2a}, R^{2b}, R^{2c}, R^{2d}, and R^{2e} is independently selected from hydrogen, methyl, ethyl, n-propyl, and i-propyl. In yet a further aspect, each of R^{2a}, R^{2b}, R^{2c}, R^{2d}, and R^{2e} is independently selected from hydrogen, methyl, and ethyl. In an even further aspect, each of R^{2a}, R^{2b}, R^{2c}, R^{2d}, and R^{2e} is independently selected from hydrogen and ethyl. In a still further aspect, each of R^{2a}, R^{2b}, R^{2c}, R^{2d}, and R^{2e} is independently selected from hydrogen and methyl.

In a further aspect, each of R^{2a}, R^{2b}, R^{2c}, R^{2d}, and R^{2e} is independently selected from hydrogen, —NH₂, —OH, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, and —N(CH₃)CH(CH₃)₂. In yet a further aspect, each of R^{2a}, R^{2b}, R^{2c}, R^{2d}, and R^{2e} is independently selected from hydrogen, —NH₂, —OH, —CH₂OH, —CH₂CH₂OH, —OCH₃, —OCH₂CH₃, —NHCH₃, —NHCH₂CH₃, —N(CH₃)₂, —N(CH₃)CH₂CH₃, and —N(CH₂CH₃)₂. In an even further aspect, each of R^{2a}, R^{2b}, R^{2c}, R^{2d}, and R^{2e} is independently selected from hydrogen, —NH₂, —OH, —CH₂OH, —OCH₃, —NHCH₃, and —N(CH₃)₂.

In a further aspect, each of R^{2a}, R^{2b}, R^{2c}, R^{2d}, and R^{2e} is independently selected from hydrogen, —NH₂, —OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, and —N(CH₃)CH(CH₃)₂. In yet a further aspect, each of R^{2a}, R^{2b}, R^{2c}, R^{2d}, and R^{2e} is independently selected from hydrogen, —NH₂, —OH, —OCH₃, —OCH₂CH₃, —NHCH₃, —NHCH₂CH₃, —N(CH₃)₂, —N(CH₃)CH₂CH₃, and —N(CH₂CH₃)₂. In an even further aspect, each of R^{2a}, R^{2b}, R^{2c}, R^{2d}, and R^{2e} is independently selected from hydrogen, —NH₂, —OH, —OCH₃, —NHCH₃, and —N(CH₃)₂.

In a further aspect, each of R^{2a}, R^{2b}, R^{2c}, R^{2d}, and R^{2e} is independently selected from hydrogen, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)₂, —N(CH₃)CH₂CH₂CH₃, and —N(CH₃)CH(CH₃)₂. In yet a further aspect, each of R^{2a}, R^{2b}, R^{2c}, R^{2d}, and R^{2e} is independently selected from hydrogen, —NH₂, —NHCH₃, —NHCH₂CH₃, —N(CH₃)₂, —N(CH₃)CH₂CH₃, and —N(CH₂CH₃)₂. In an even further aspect, each of R^{2a}, R^{2b}, R^{2c}, R^{2d}, and R^{2e} is independently selected from hydrogen, —NH₂, —NHCH₃, and —N(CH₃)₂.

In a further aspect, each of R^{2a}, R^{2b}, R^{2c}, R^{2d}, and R^{2e} is independently selected from hydrogen, —OH, —OCH₃, —OCH₂CH₃, —OCH₂CH₂CH₃, and —OCH(CH₃)₂. In yet a further aspect, each of R^{2a}, R^{2b}, R^{2c}, R^{2d}, and R^{2e} is independently selected from hydrogen, —OH, —OCH₃, and —OCH₂CH₃. In an even further aspect, each of R^{2a}, R^{2b}, R^{2c}, R^{2d}, and R^{2e} is independently selected from hydrogen, —OH, and —OCH₃.

In a further aspect, at least one of R^{2a}, R^{2b}, R^{2c}, R^{2d}, and R^{2e} is C1-C4 alkoxy or C1-C4 haloalkoxy. In a still further aspect, at least two of R^{2a}, R^{2b}, R^{2c}, R^{2d}, and R^{2e} are C1-C4 alkoxy or C1-C4 haloalkoxy. In yet a further aspect, at least three of R^{2a}, R^{2b}, R^{2c}, R^{2d}, and R^{2e} are C1-C4 alkoxy or C1-C4 haloalkoxy.

In a further aspect, at least one of R^{2a}, R^{2b}, R^{2c}, R^{2d}, and R^{2e} is C1-C4 alkoxy. In a still further aspect, at least two of R^{2a}, R^{2b}, R^{2c}, R^{2d}, and R^{2e} are C1-C4 alkoxy. In yet a further aspect, at least three of R^{2a}, R^{2b}, R^{2c}, R^{2d}, and R^{2e} are C1-C4 alkoxy.

In a further aspect, at least one of R^{2a}, R^{2b}, R^{2c}, R^{2d}, and R^{2e} is methoxy. In a still further aspect, at least two of R^{2a}, R^{2b}, R^{2c}, R^{2d}, and R^{2e} are methoxy. In yet a further aspect, at least three of R^{2a}, R^{2b}, R^{2c}, R^{2d}, and R^{2e} are methoxy.

In a further aspect, each of R^{2b}, R^{2c}, and R^{2d} is methoxy.

d. R³ Groups

In one aspect, R³, when present, is selected from C1-C8 alkyl, (CH₂)_{q}Cy¹, and Cy¹. In a further aspect, R³, when present, is selected from C1-C4 alkyl, (CH₂)_{q}Cy¹, and Cy¹. In a still further aspect, R³, when present, is selected from methyl, ethyl, n-propyl, i-propyl, (CH₂)_{q}Cy¹, and Cy¹.

In a further aspect, R³, when present, is selected from (CH₂)_{q}Cy¹ and Cy¹. In a still further aspect, R³, when present, (CH₂)_{q}Cy¹. In yet a further aspect, R³, when present, Cy¹.

In a further aspect, R³, when present, is selected from CH₂Cy¹ and Cy¹. In a still further aspect, R³, when present, is CH₂Cy¹.

In a further aspect, R³, when present, is C1-C8 alkyl. In a still further aspect, R³, when present, is C1-C4 alkyl. In yet a further aspect, R³, when present, is selected from methyl, ethyl, n-propyl, and i-propyl. In an even further aspect, R³, when present, is selected from methyl and ethyl. In a still further aspect, R³, when present, is selected from n-propyl, and i-propyl. In yet a further aspect, R³, when present, is ethyl. In an even further aspect, R³, when present, is methyl.

e. R^{20A} and R^{20B} Groups

In one aspect, each of R^{20a} and R^{20b}, when present, is independently selected from hydrogen and C1-C4 alkyl. In a further aspect, each of R^{20a} and R^{20b}, when present, is independently selected from hydrogen, methyl, ethyl, n-propyl, and i-propyl. In a still further aspect, each of R^{20a} and R^{20b}, when present, is independently selected from hydrogen, methyl, and ethyl. In yet a further aspect, each of R^{20a} and R^{20b}, when present, is independently selected from hydrogen and ethyl. In an even further aspect, each of R^{20a} and R^{20b}, when present, is independently selected from hydrogen and methyl. In a still further aspect, each of R^{20a} and R^{20b}, when present, is hydrogen.

In a further aspect, each of R^{20a} and R^{20b}, when present, is independently C1-C4 alkyl. In a still further aspect, each of R^{20a} and R^{20b}, when present, is independently selected from methyl, ethyl, n-propyl, and i-propyl. In yet a further aspect, each of R^{20a} and R^{20b}, when present, is independently selected from methyl and ethyl. In an even further aspect, each of R^{20a} and R^{20b}, when present, is ethyl. In a still further aspect, each of R^{20a} and R^{20b}, when present, is methyl.

In a further aspect, R^{20a}, when present, is hydrogen and R^{20b}, when present, is selected from hydrogen and C1-C4 alkyl. In a still further aspect, R^{20a}, when present, is hydrogen and R^{20b}, when present, is selected from hydrogen, methyl, ethyl, n-propyl, and i-propyl. In yet a further aspect, R^{20a}, when present, is hydrogen and R^{20b}, when present, is selected from hydrogen, methyl, and ethyl. In an even further aspect, R^{20a}, when present, is hydrogen and R^{20b}, when present, is selected from hydrogen and ethyl. In a still further aspect, R^{20a}, when present, is hydrogen and R^{20b}, when present, is selected from hydrogen and methyl.

In a further aspect, R^{20b}, when present, is hydrogen and R^{20a}, when present, is selected from hydrogen and C1-C4 alkyl. In a still further aspect, R^{20b}, when present, is hydrogen and R^{20a}, when present, is selected from hydrogen, methyl, ethyl, n-propyl, and i-propyl. In yet a further aspect, R^{20b}, when present, is hydrogen and R^{20a}, when present, is selected from hydrogen, methyl, and ethyl. In an even further aspect, R^{20b}, when present, is hydrogen and R^{20a}, when present, is selected from hydrogen and ethyl. In a still further aspect, R^{20b}, when present, is hydrogen and R^{20a}, when present, is selected from hydrogen and methyl.

f. Ar¹ Groups

In one aspect, Ar¹ is selected from C6-aryl and pyridinyl, and is substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a further aspect, Ar¹ is selected from C6-aryl and pyridinyl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Ar¹ is selected from C6-aryl and pyridinyl, and is substituted with 0, 1, or 2 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In yet a further aspect, Ar¹ is selected from C6-aryl and pyridinyl, and is substituted with 0 or 1 group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In an even further aspect, Ar¹ is selected from C6-aryl and pyridinyl, and is monosubstituted with a group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Ar¹ is selected from C6-aryl and pyridinyl, and is unsubstituted.

In a further aspect, Ar¹ is selected from C6-aryl and pyridinyl, and is substituted with 0, 1, 2, or 3 C1-C4 alkoxy groups. In a still further aspect, Ar¹ is selected from C6-aryl and pyridinyl, and is substituted with 1, 2, or 3 C1-C4 alkoxy groups. In yet a further aspect, Ar¹ is selected from C6-aryl and pyridinyl, and is substituted with 0, 1, 2, or 3 methoxy groups. In an even further aspect, Ar¹ is selected from C6-aryl and pyridinyl, and is substituted with 1, 2, or 3 methoxy groups.

In various aspects, Ar¹ is C6-aryl substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a further aspect, Ar¹ is C6-aryl substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Ar¹ is C6-aryl substituted with 0, 1, or 2 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In yet a further aspect, Ar¹ is C6-aryl substituted with 0 or 1 group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In an even further aspect, Ar¹ is C6-aryl monosubstituted with a group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Ar¹ is unsubstituted C6-aryl.

In a further aspect, Ar¹ is C6-aryl substituted with 0, 1, 2, or 3 C1-C4 alkoxy groups. In a still further aspect, Ar¹ is C6-aryl substituted with 1, 2, or 3 C1-C4 alkoxy groups. In yet a further aspect, Ar¹ is C6-aryl substituted with 0, 1, 2, or 3 methoxy groups. In an even further aspect, Ar¹ is C6-aryl substituted with 1, 2, or 3 methoxy groups.

In various aspects, Ar¹ is pyridinyl substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a further aspect, Ar¹ is pyridinyl substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Ar¹ is pyridinyl substituted with 0, 1, or 2 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In yet a further aspect, Ar¹ is pyridinyl substituted with 0 or 1 group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In an even further aspect, Ar¹ is pyridinyl monosubstituted with a group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Ar¹ is unsubstituted pyridinyl.

In a further aspect, Ar¹ is pyridinyl substituted with 0, 1, 2, or 3 C1-C4 alkoxy groups. In a still further aspect, Ar¹ is pyridinyl substituted with 1, 2, or 3 C1-C4 alkoxy groups. In yet a further aspect, Ar¹ is pyridinyl substituted with 0, 1, 2, or 3 methoxy groups. In an even further aspect, Ar¹ is pyridinyl substituted with 1, 2, or 3 methoxy groups.

g. Cy¹ Groups

In one aspect, Cy¹ is selected from cyclohexyl, a 6-membered heterocycloalkyl, and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a further aspect, Cy¹ is selected from cyclohexyl, a 6-membered heterocycloalkyl, and a 6-membered monocyclic aryl, and is substituted with 0, 1, or 2 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Cy¹ is selected from cyclohexyl, a 6-membered heterocycloalkyl, and a 6-membered monocyclic aryl, and is substituted with 0 or 1 group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In yet a further aspect, Cy¹ is selected from cyclohexyl, a 6-membered heterocycloalkyl, and a 6-membered monocyclic aryl, and is monosubstituted with a group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In an even further aspect, Cy¹ is selected from cyclohexyl, a 6-membered heterocycloalkyl, and a 6-membered monocyclic aryl, and is unsubstituted.

In various aspects, Cy¹ is selected from cyclohexyl and 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a further aspect, Cy¹ is selected from cyclohexyl and 6-membered monocyclic aryl, and is substituted with 0, 1, or 2 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Cy¹ is selected from cyclohexyl and 6-membered monocyclic aryl, and is substituted with 0 or 1 group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In yet a further aspect, Cy¹ is selected from cyclohexyl and 6-membered monocyclic aryl, and is monosubstituted with a group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In an even further aspect, Cy¹ is selected from cyclohexyl and 6-membered monocyclic aryl, and is unsubstituted.

In a further aspect, Cy¹ is cyclohexyl substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Cy¹ is cyclohexyl substituted with 0, 1, or 2 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In yet a further aspect, Cy¹ is cyclohexyl substituted with 0 or 1 group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In an even further aspect, Cy¹ is cyclohexyl monosubstituted with a group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Cy¹ is unsubstituted cyclohexyl.

In a further aspect, Cy¹ is a 6-membered heterocycloalkyl substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. Examples of 6-membered heterocycloalkyls include, but are not limited to, piperidinyl, piperidino, piperazinyl, piperazino, morpholinyl, morpholino, thiomorpholinyl, and thiomorpholino. In a still further aspect, Cy¹ is a 6-membered heterocycloalkyl substituted with 0, 1, or 2 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In yet a further aspect, Cy¹ is a 6-membered heterocycloalkyl substituted with 0 or 1 group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In an even further aspect, Cy¹ is a 6-membered heterocycloalkyl monosubstituted with a group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Cy¹ is unsubstituted 6-membered heterocycloalkyl substituted.

In a further aspect, Cy¹ is a 6-membered monocyclic aryl substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Cy¹ is a 6-membered monocyclic aryl substituted with 0, 1, or 2 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In yet a further aspect, Cy¹ is a 6-membered monocyclic aryl substituted with 0 or 1 group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In an even further aspect, Cy¹ is a 6-membered monocyclic aryl monosubstituted with a group selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a still further aspect, Cy¹ is unsubstituted 6-membered monocyclic aryl.

### 2. Example Compounds

In one aspect, a compound can be present as the following structure:

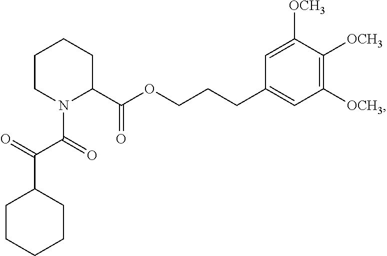 [SMILES: COc1cc(CCCOC(=O)C2CCCCN2C(=O)C(=O)C2CCCCC2)cc(OC)c1OC]

or a pharmaceutically acceptable salt thereof.

In one aspect, a compound can be present as the following structure:

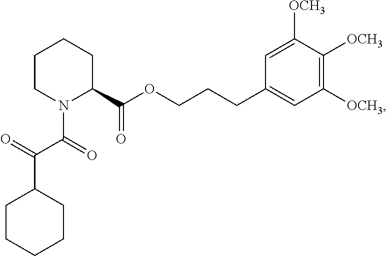 [SMILES: COc1cc(CCCOC(=O)[C@@H]2CCCCN2C(=O)C(=O)C2CCCCC2)cc(OC)c1OC]

or a pharmaceutically acceptable salt thereof.

In one aspect, a compound can be present as one or more of the following structures:

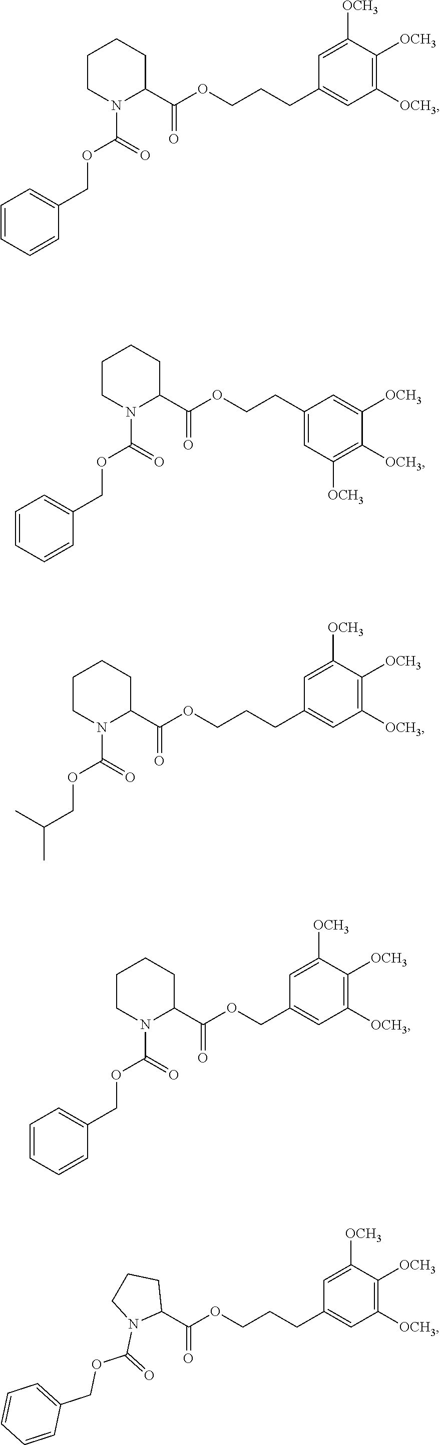 [SMILES: COc1cc(CCCOC(=O)C2CCCCN2C(=O)OCc2ccccc2)cc(OC)c1OC ; COc1cc(CCOC(=O)C2CCCCN2C(=O)OCc2ccccc2)cc(OC)c1OC ; COc1cc(CCCOC(=O)C2CCCCN2C(=O)OCC(C)C)cc(OC)c1OC ; COc1cc(COC(=O)C2CCCCN2C(=O)OCc2ccccc2)cc(OC)c1OC ; COc1cc(CCCOC(=O)C2CCCN2C(=O)OCc2ccccc2)cc(OC)c1OC]

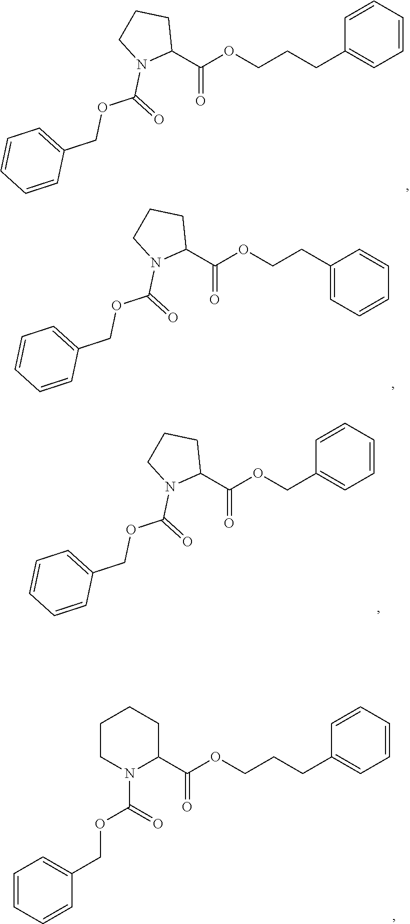 [SMILES: O=C(OCCCc1ccccc1)C1CCCN1C(=O)OCc1ccccc1 ; O=C(OCCc1ccccc1)C1CCCN1C(=O)OCc1ccccc1 ; O=C(OCc1ccccc1)C1CCCN1C(=O)OCc1ccccc1 ; O=C(OCCCc1ccccc1)C1CCCCN1C(=O)OCc1ccccc1]

or a pharmaceutically acceptable salt thereof.

In one aspect, a compound can be present as one or more of the following structures:

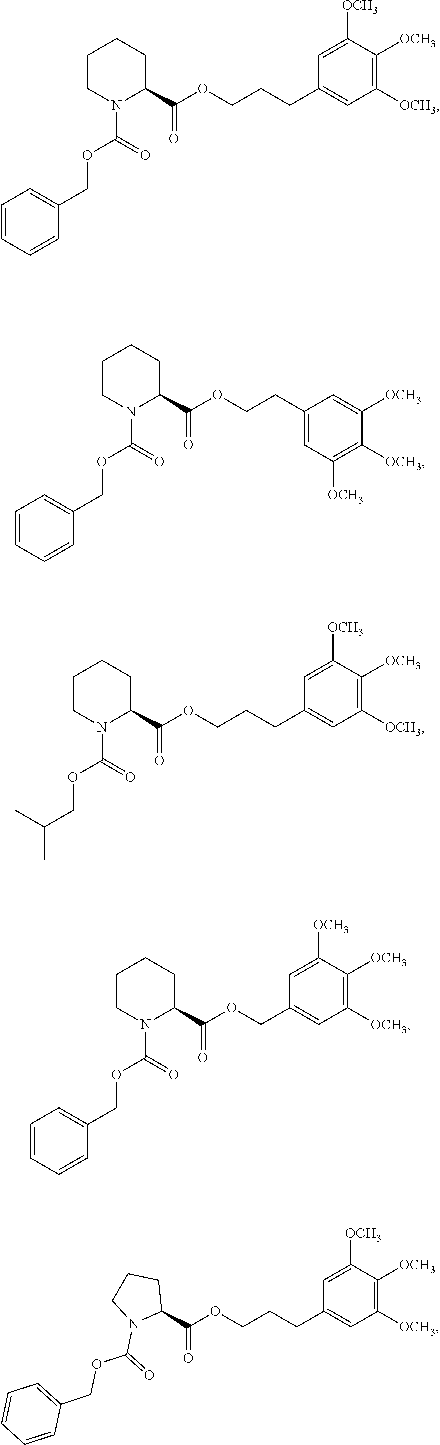 [SMILES: COc1cc(CCCOC(=O)[C@@H]2CCCCN2C(=O)OCc2ccccc2)cc(OC)c1OC ; COc1cc(CCOC(=O)[C@@H]2CCCCN2C(=O)OCc2ccccc2)cc(OC)c1OC ; COc1cc(CCCOC(=O)[C@@H]2CCCCN2C(=O)OCC(C)C)cc(OC)c1OC ; COc1cc(COC(=O)[C@@H]2CCCCN2C(=O)OCc2ccccc2)cc(OC)c1OC ; COc1cc(CCCOC(=O)[C@@H]2CCCN2C(=O)OCc2ccccc2)cc(OC)c1OC]

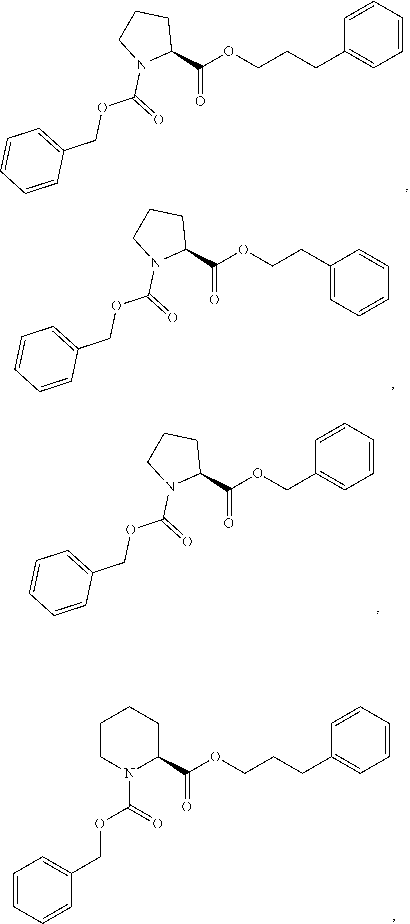 [SMILES: O=C(OCCCc1ccccc1)[C@@H]1CCCN1C(=O)OCc1ccccc1 ; O=C(OCCc1ccccc1)[C@@H]1CCCN1C(=O)OCc1ccccc1 ; O=C(OCc1ccccc1)[C@@H]1CCCN1C(=O)OCc1ccccc1 ; O=C(OCCCc1ccccc1)[C@@H]1CCCCN1C(=O)OCc1ccccc1]

or a pharmaceutically acceptable salt thereof.

In one aspect, a compound can be present as the following structure:

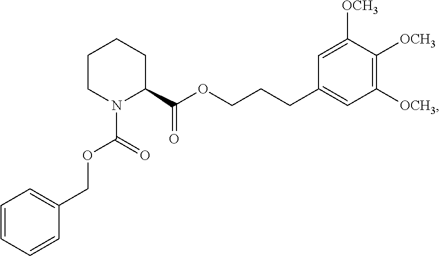 [SMILES: COc1cc(CCCOC(=O)[C@@H]2CCCCN2C(=O)OCc2ccccc2)cc(OC)c1OC]

or a pharmaceutically acceptable salt thereof.

In one aspect, a compound can be present as the following structure:

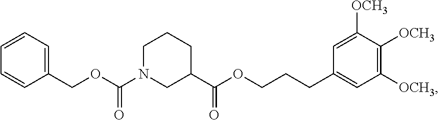 [SMILES: COc1cc(CCCOC(=O)C2CCCN(C(=O)OCc3ccccc3)C2)cc(OC)c1OC]

or a pharmaceutically acceptable salt thereof.

In one aspect, a compound can be present as the following structure:

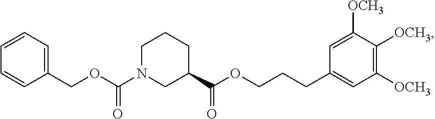 [SMILES: COc1cc(CCCOC(=O)[C@@H]2CCCN(C(=O)OCc3ccccc3)C2)cc(OC)c1OC]

or a pharmaceutically acceptable salt thereof.

### 3. Prophetic Compound Examples

The following compound examples are prophetic, and can be prepared using the synthesis methods described herein above and other general methods as needed as would be known to one skilled in the art. It is anticipated that the prophetic compounds would be active as proteasome inhibitors, and such activity can be determined using the assay methods described herein.

In one aspect, a compound can be selected from:

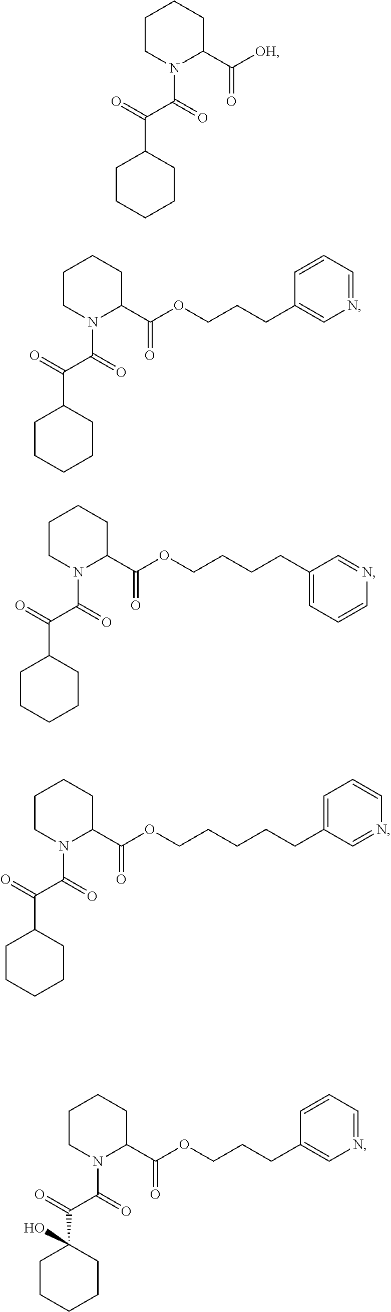

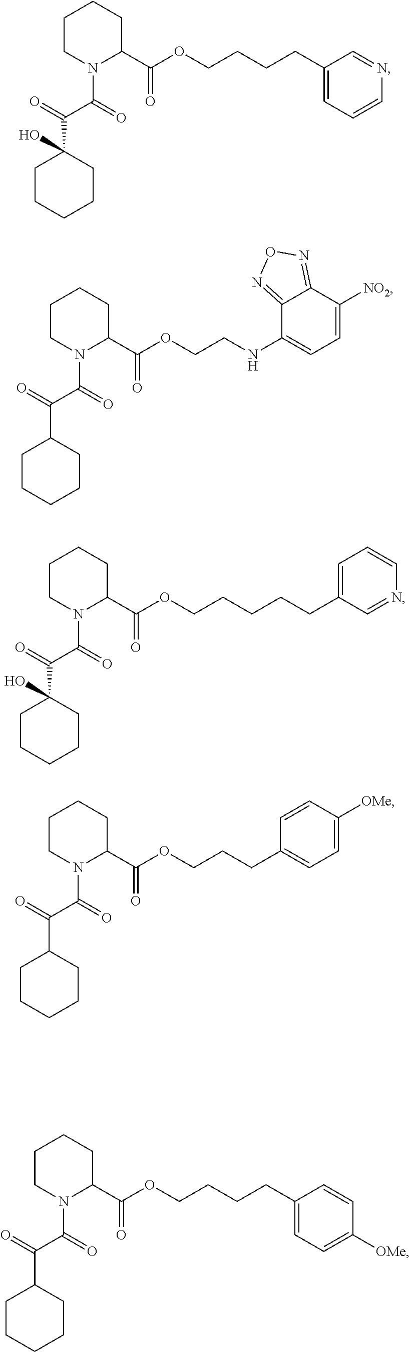

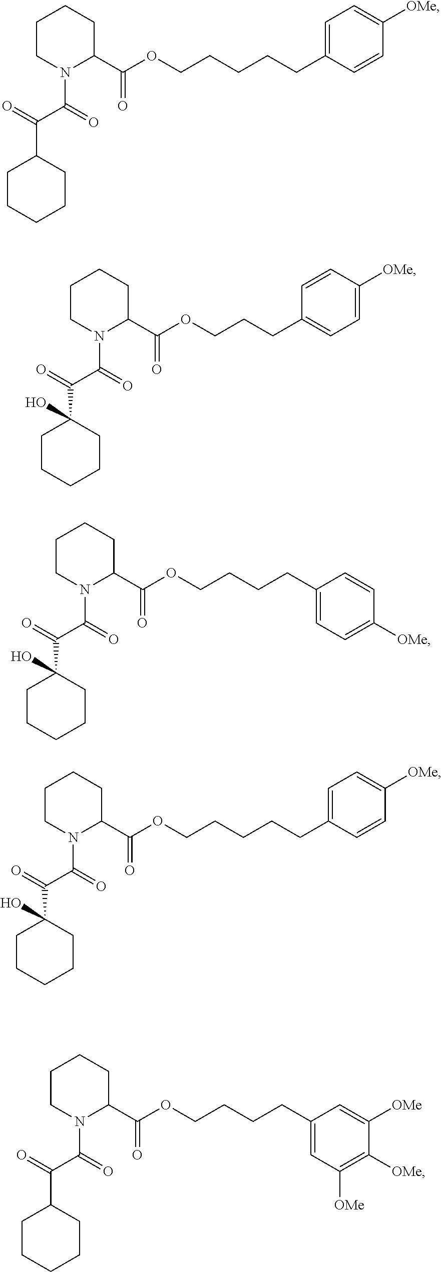

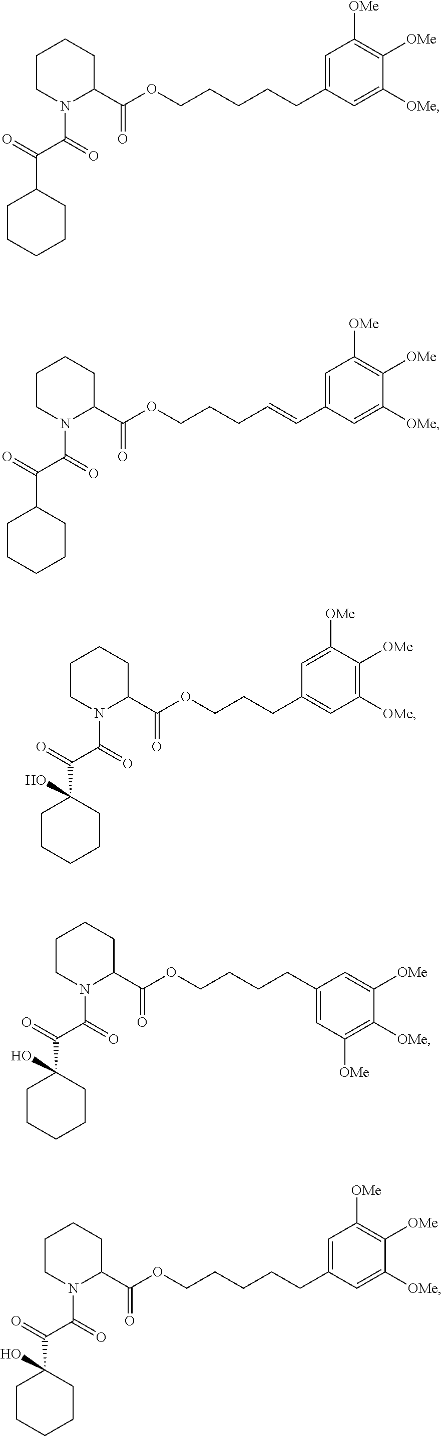

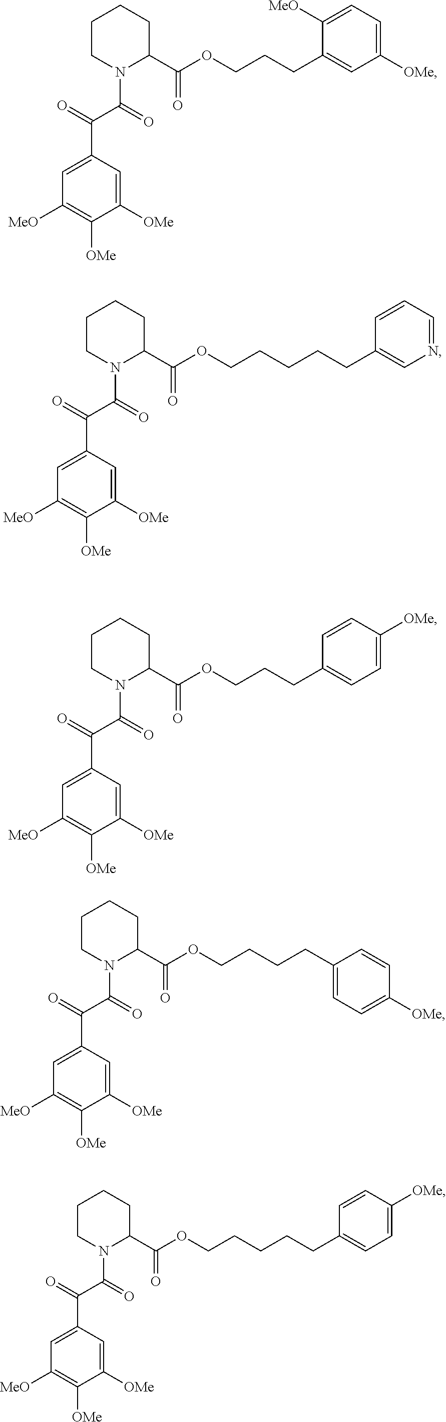

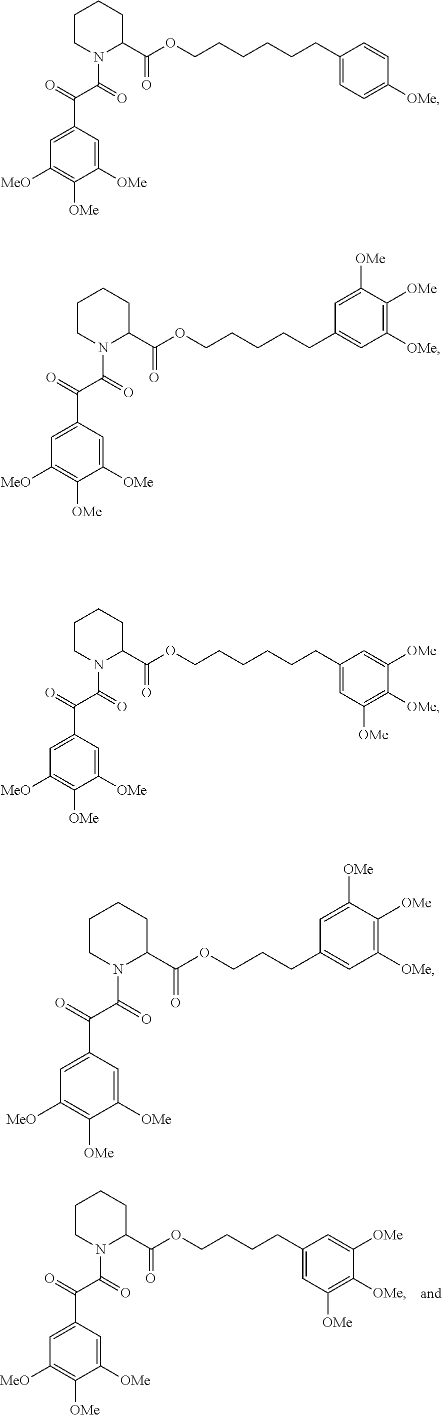

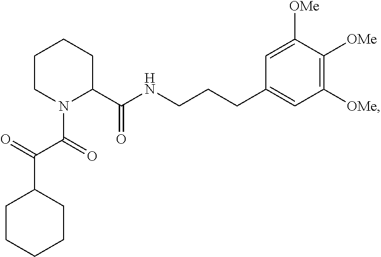 [SMILES: COc1cc(CCCNC(=O)C2CCCCN2C(=O)C(=O)C2CCCCC2)cc(OC)c1OC]

or a pharmaceutically acceptable salt thereof.

In one aspect, a compound can be selected from:

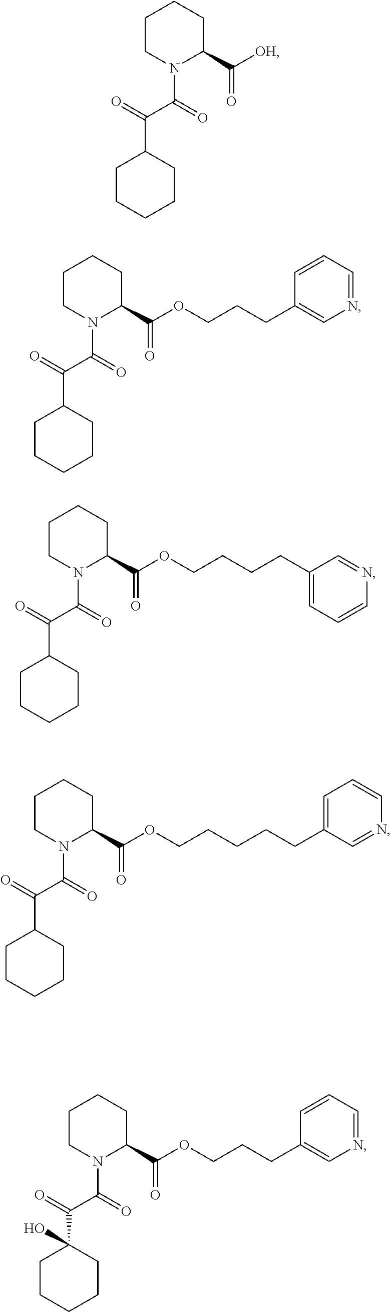

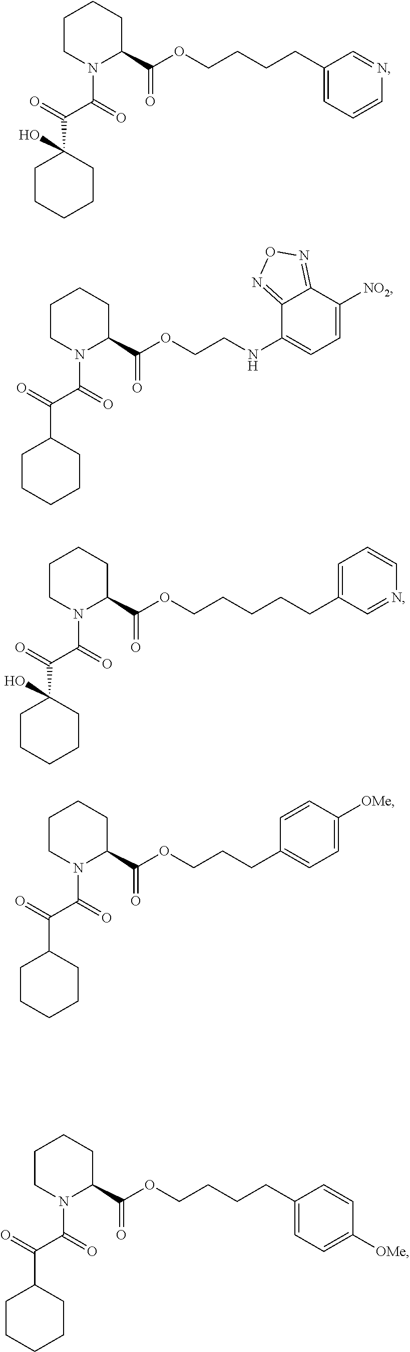

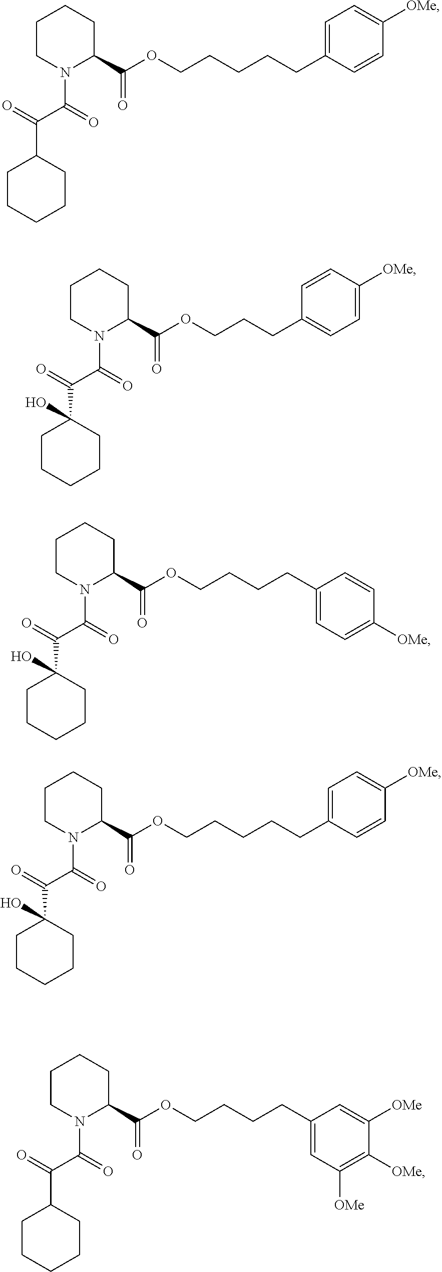

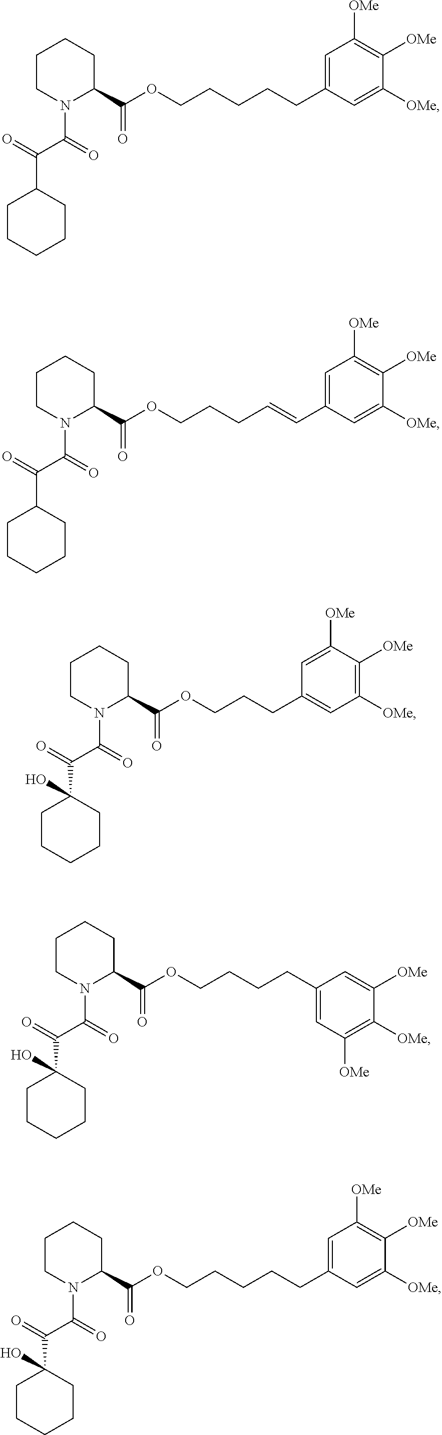

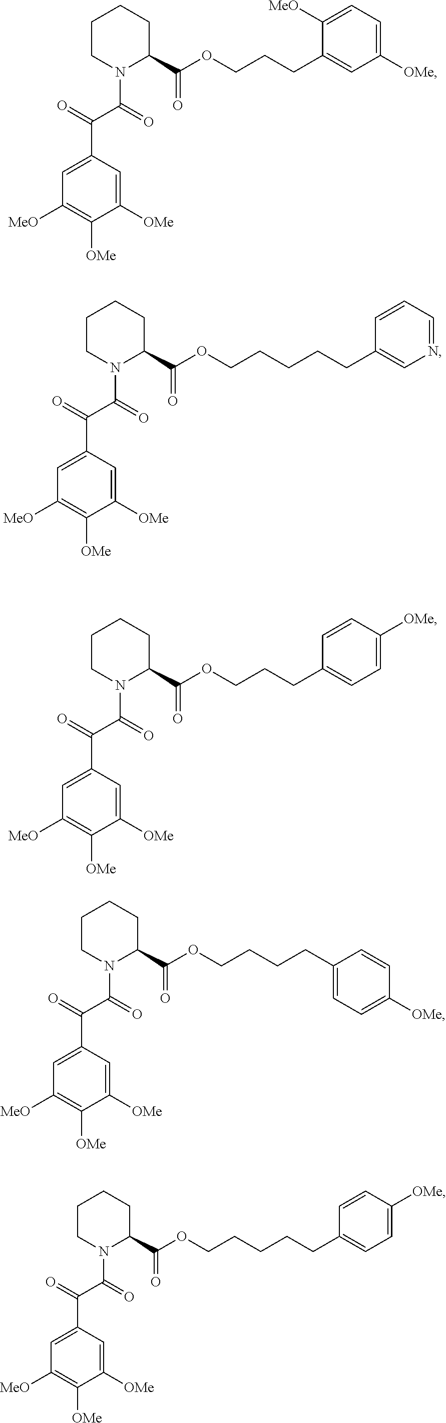

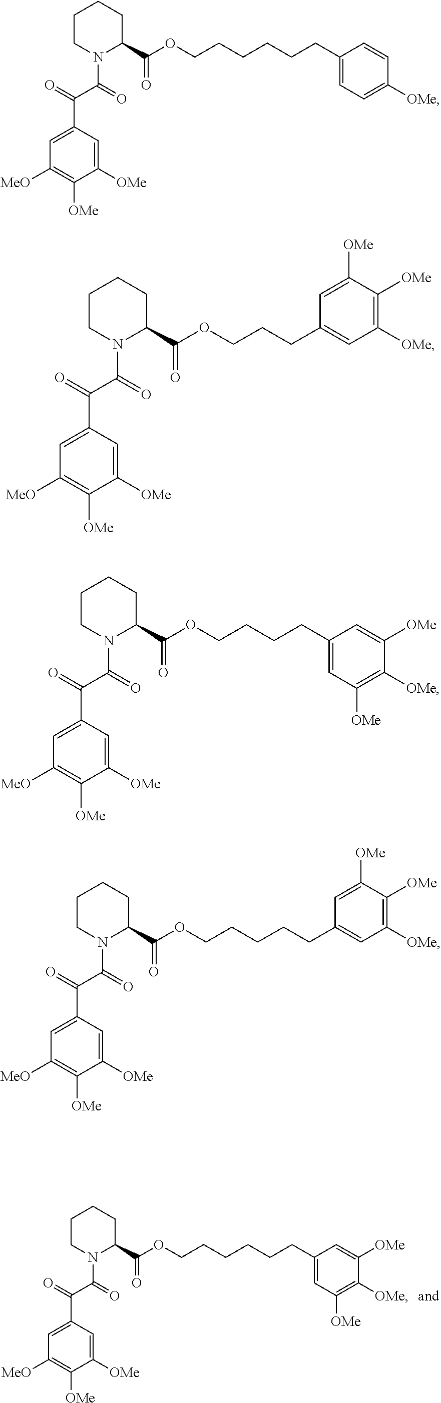 [SMILES: COc1ccc(CCCCCCOC(=O)[C@@H]2CCCCN2C(=O)C(=O)c2cc(OC)c(OC)c(OC)c2)cc1 ; COc1cc(CCCOC(=O)[C@@H]2CCCCN2C(=O)C(=O)c2cc(OC)c(OC)c(OC)c2)cc(OC)c1OC ; COc1cc(CCCCOC(=O)[C@@H]2CCCCN2C(=O)C(=O)c2cc(OC)c(OC)c(OC)c2)cc(OC)c1OC ; COc1cc(CCCCCOC(=O)[C@@H]2CCCCN2C(=O)C(=O)c2cc(OC)c(OC)c(OC)c2)cc(OC)c1OC ; COc1cc(CCCCCCOC(=O)[C@@H]2CCCCN2C(=O)C(=O)c2cc(OC)c(OC)c(OC)c2)cc(OC)c1OC]

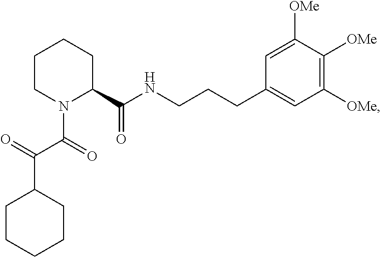 [SMILES: COc1cc(CCCNC(=O)[C@@H]2CCCCN2C(=O)C(=O)C2CCCCC2)cc(OC)c1OC]

or a pharmaceutically acceptable salt thereof.

In one aspect, a compound can be selected from:

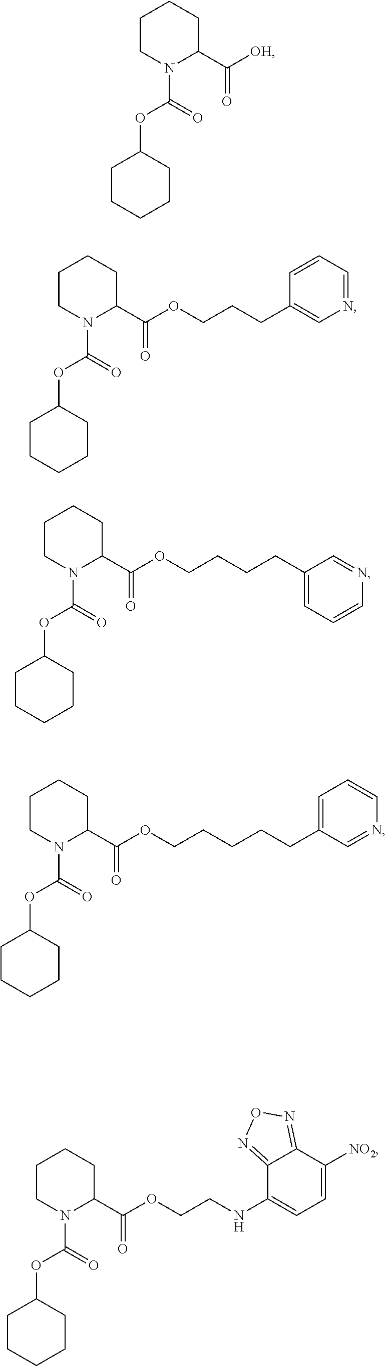

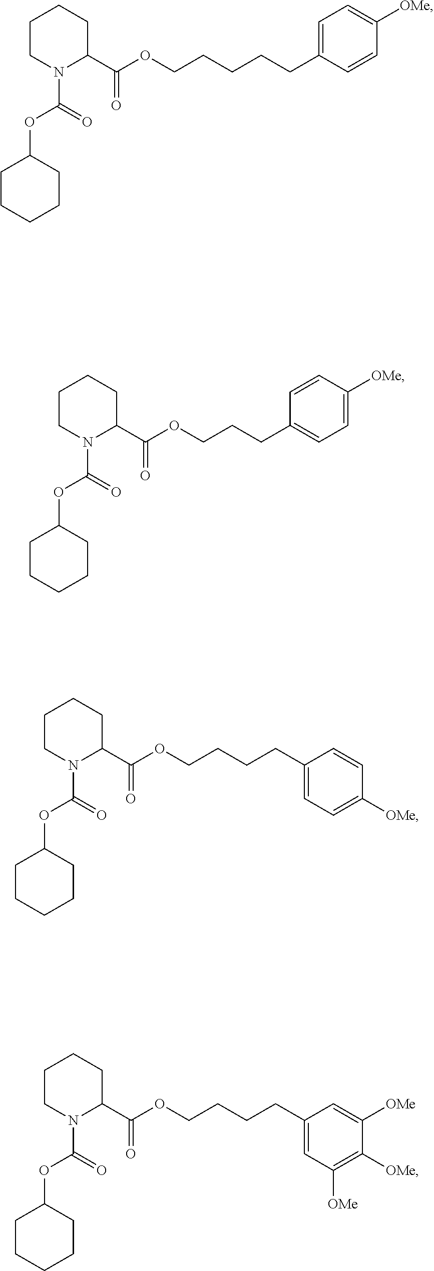 [SMILES: COc1ccc(CCCCCOC(=O)C2CCCCN2C(=O)OC2CCCCC2)cc1 ; COc1ccc(CCCOC(=O)C2CCCCN2C(=O)OC2CCCCC2)cc1 ; COc1ccc(CCCCOC(=O)C2CCCCN2C(=O)OC2CCCCC2)cc1 ; COc1cc(CCCCOC(=O)C2CCCCN2C(=O)OC2CCCCC2)cc(OC)c1OC]

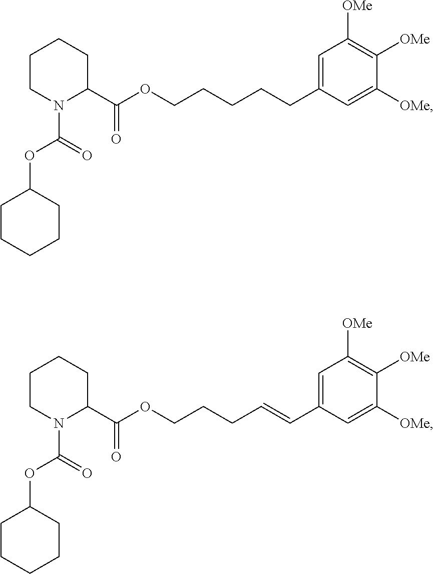 [SMILES: COc1cc(CCCCCOC(=O)C2CCCCN2C(=O)OC2CCCCC2)cc(OC)c1OC ; COc1cc(/C=C/CCCOC(=O)C2CCCCN2C(=O)OC2CCCCC2)cc(OC)c1OC]

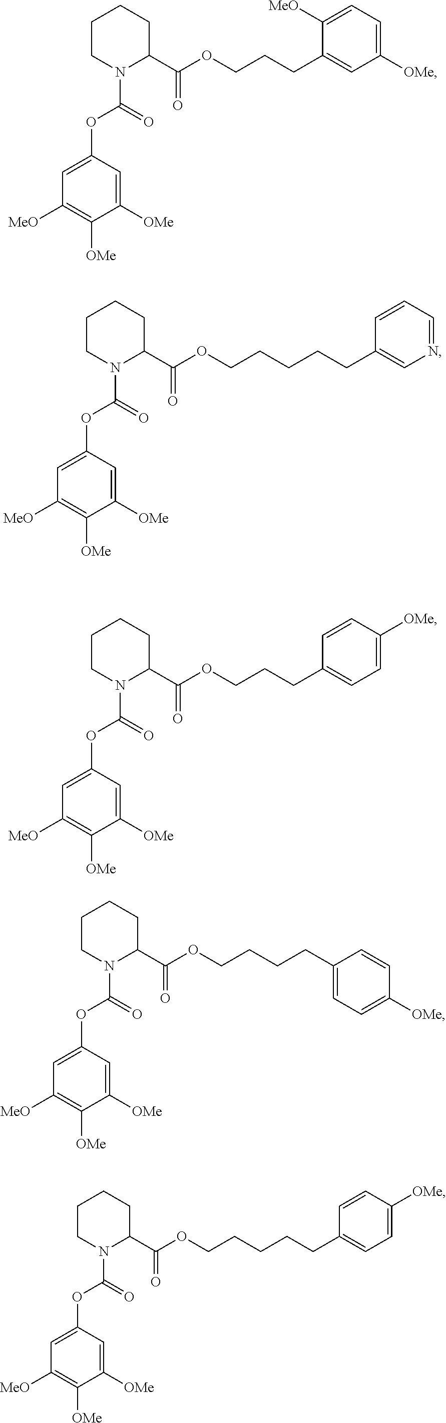

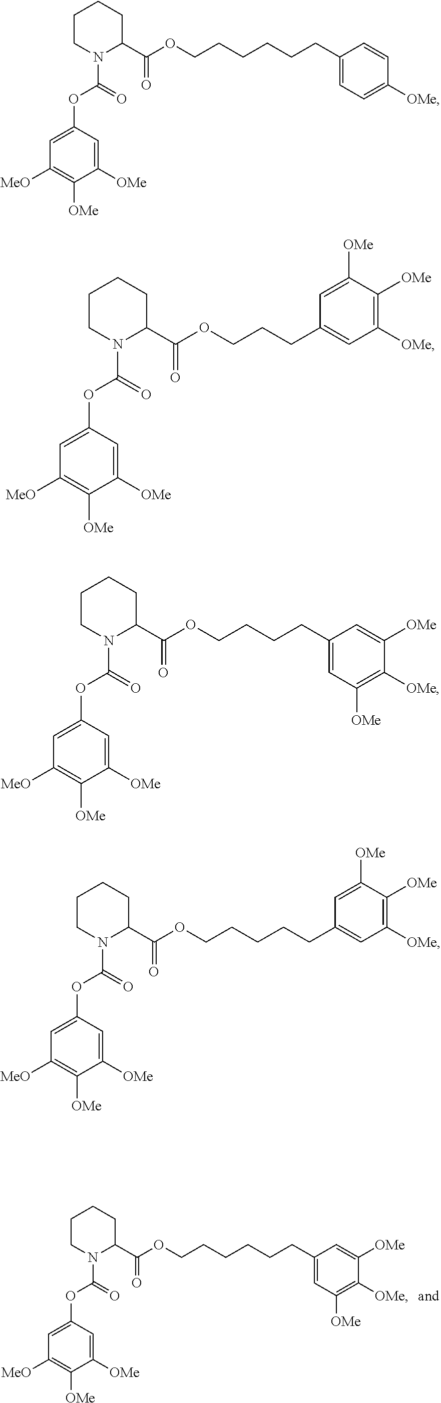 [SMILES: COc1ccc(CCCCCCOC(=O)C2CCCCN2C(=O)Oc2cc(OC)c(OC)c(OC)c2)cc1 ; COc1cc(CCCOC(=O)C2CCCCN2C(=O)Oc2cc(OC)c(OC)c(OC)c2)cc(OC)c1OC ; COc1cc(CCCCOC(=O)C2CCCCN2C(=O)Oc2cc(OC)c(OC)c(OC)c2)cc(OC)c1OC ; COc1cc(CCCCCOC(=O)C2CCCCN2C(=O)Oc2cc(OC)c(OC)c(OC)c2)cc(OC)c1OC ; COc1cc(CCCCCCOC(=O)C2CCCCN2C(=O)Oc2cc(OC)c(OC)c(OC)c2)cc(OC)c1OC]

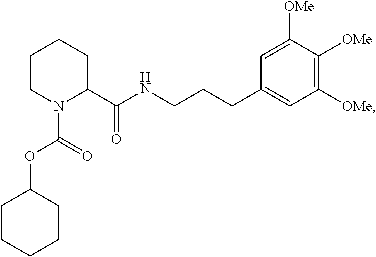 [SMILES: COc1cc(CCCNC(=O)C2CCCCN2C(=O)OC2CCCCC2)cc(OC)c1OC]

or a pharmaceutically acceptable salt thereof.

In one aspect, a compound can be selected from:

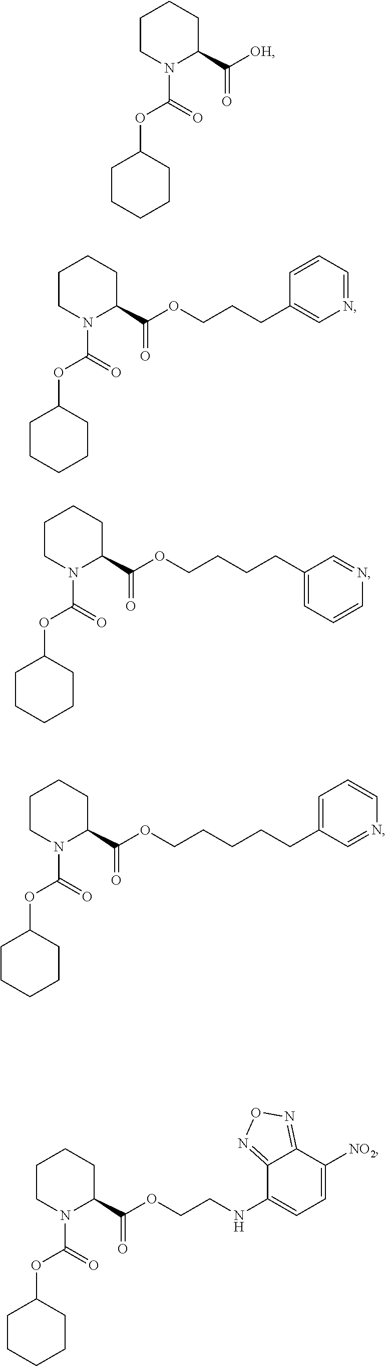

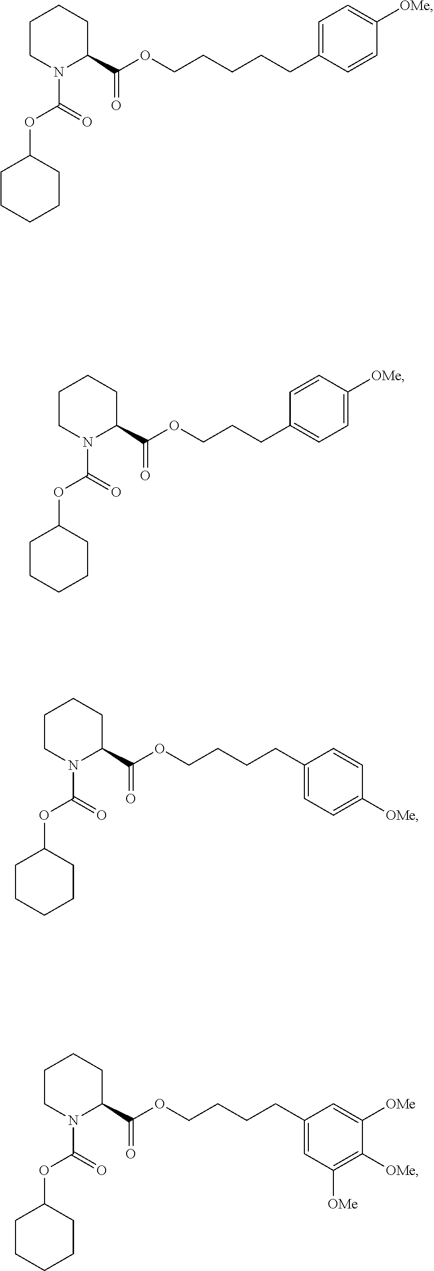 [SMILES: COc1ccc(CCCCCOC(=O)[C@@H]2CCCCN2C(=O)OC2CCCCC2)cc1 ; COc1ccc(CCCOC(=O)[C@@H]2CCCCN2C(=O)OC2CCCCC2)cc1 ; COc1ccc(CCCCOC(=O)[C@@H]2CCCCN2C(=O)OC2CCCCC2)cc1 ; COc1cc(CCCCOC(=O)[C@@H]2CCCCN2C(=O)OC2CCCCC2)cc(OC)c1OC]

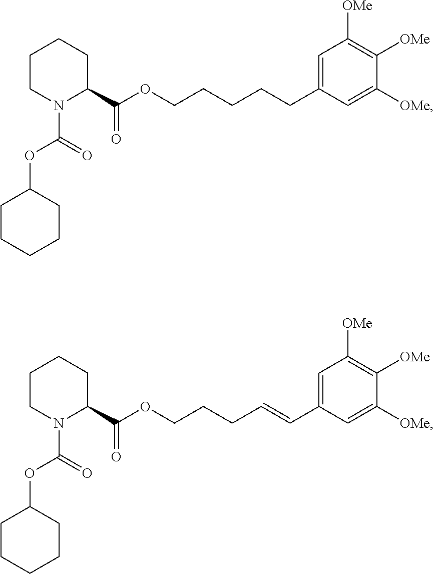 [SMILES: COc1cc(CCCCCOC(=O)[C@@H]2CCCCN2C(=O)OC2CCCCC2)cc(OC)c1OC ; COc1cc(/C=C/CCCOC(=O)[C@@H]2CCCCN2C(=O)OC2CCCCC2)cc(OC)c1OC]

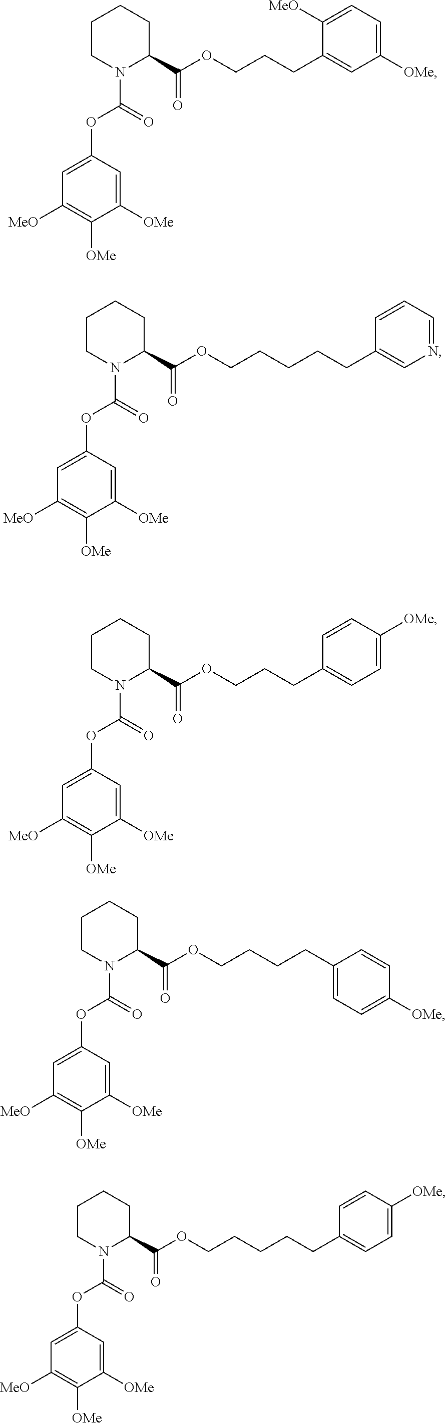

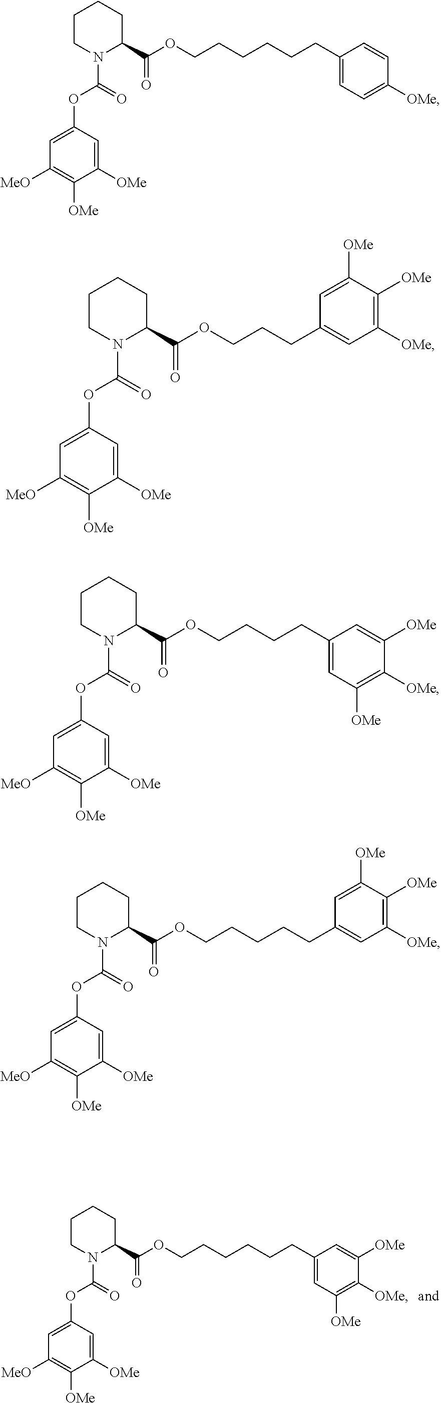 [SMILES: COc1ccc(CCCCCCOC(=O)[C@@H]2CCCCN2C(=O)Oc2cc(OC)c(OC)c(OC)c2)cc1 ; COc1cc(CCCOC(=O)[C@@H]2CCCCN2C(=O)Oc2cc(OC)c(OC)c(OC)c2)cc(OC)c1OC ; COc1cc(CCCCOC(=O)[C@@H]2CCCCN2C(=O)Oc2cc(OC)c(OC)c(OC)c2)cc(OC)c1OC ; COc1cc(CCCCCOC(=O)[C@@H]2CCCCN2C(=O)Oc2cc(OC)c(OC)c(OC)c2)cc(OC)c1OC ; COc1cc(CCCCCCOC(=O)[C@@H]2CCCCN2C(=O)Oc2cc(OC)c(OC)c(OC)c2)cc(OC)c1OC]

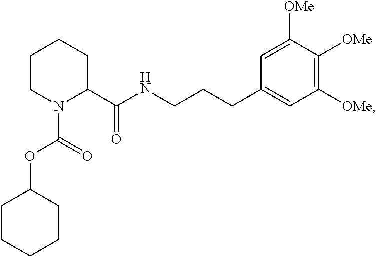 [SMILES: COc1cc(CCCNC(=O)C2CCCCN2C(=O)OC2CCCCC2)cc(OC)c1OC]

or a pharmaceutically acceptable salt thereof.

In one aspect, a compound can be selected from:

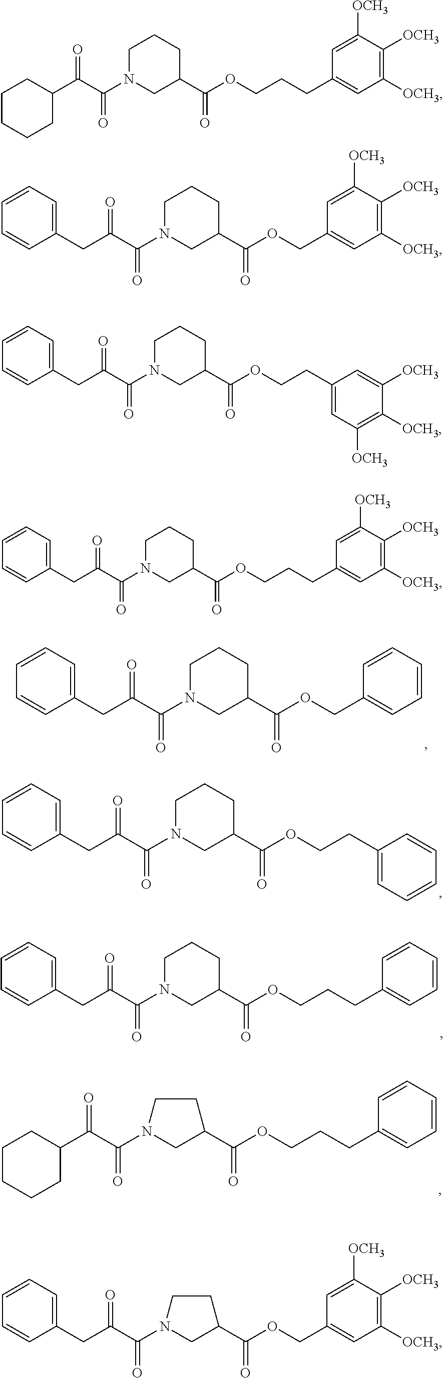

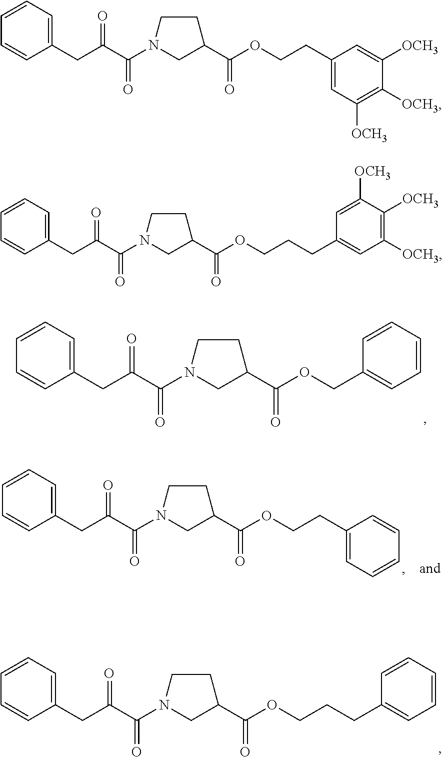

or a pharmaceutically acceptable salt thereof.

In one aspect, a compound can be selected from:

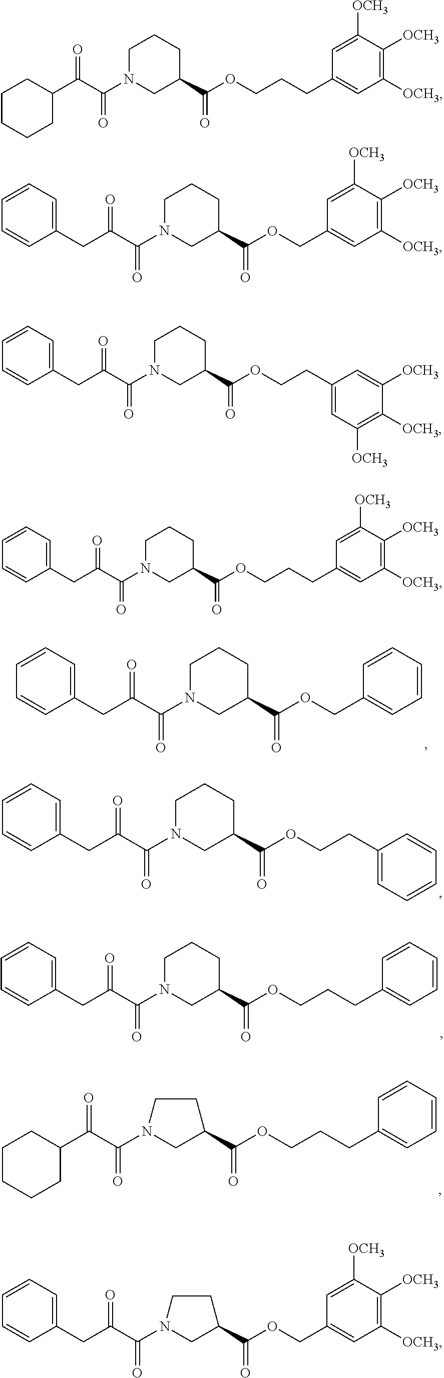

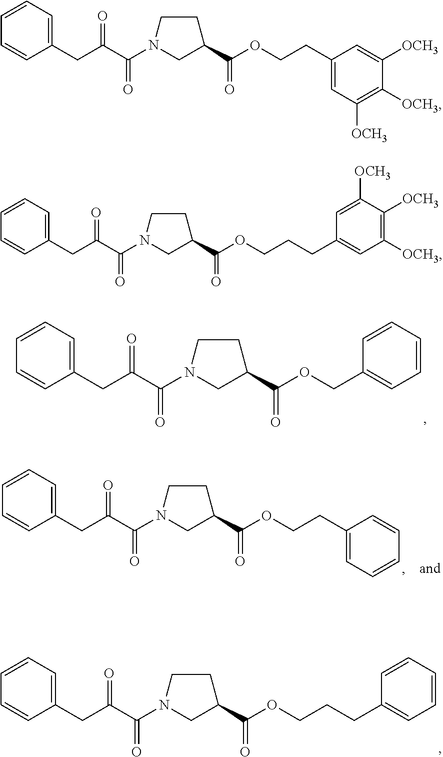

or a pharmaceutically acceptable salt thereof.

In one aspect, a compound can be selected from:

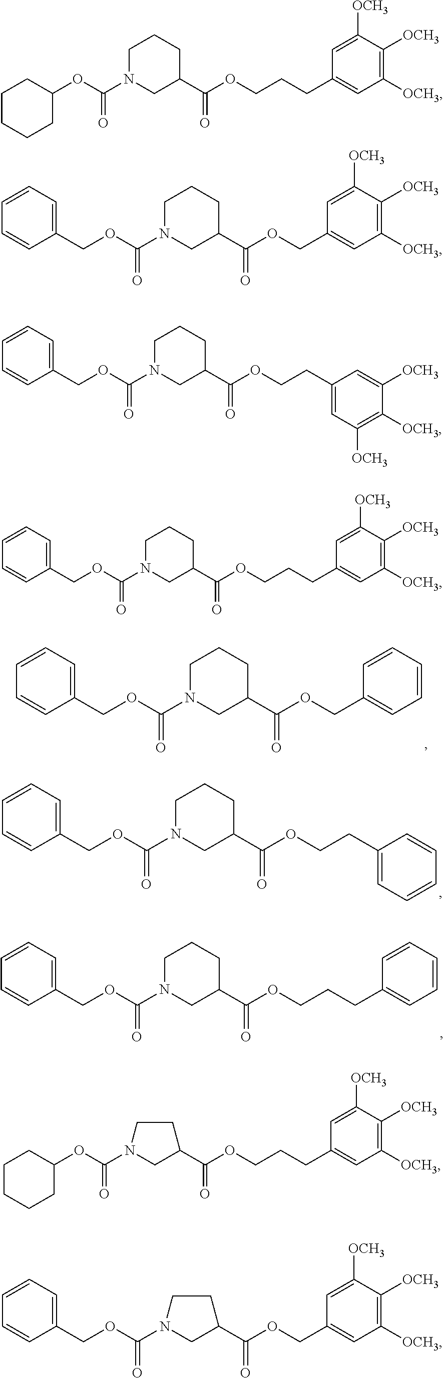

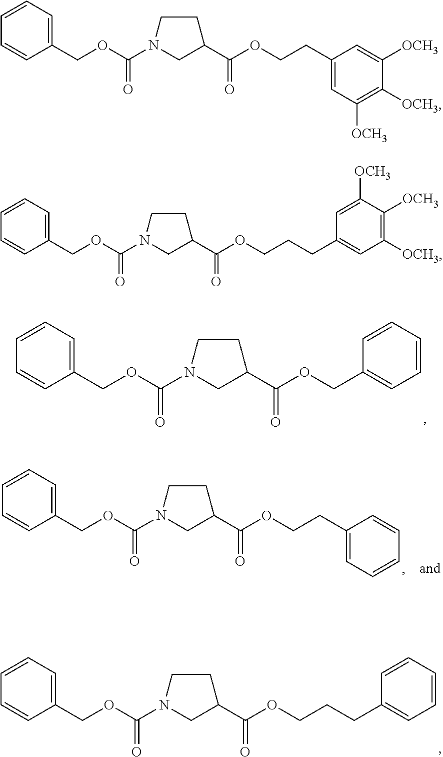

or a pharmaceutically acceptable salt thereof.

In one aspect, a compound can be selected from:

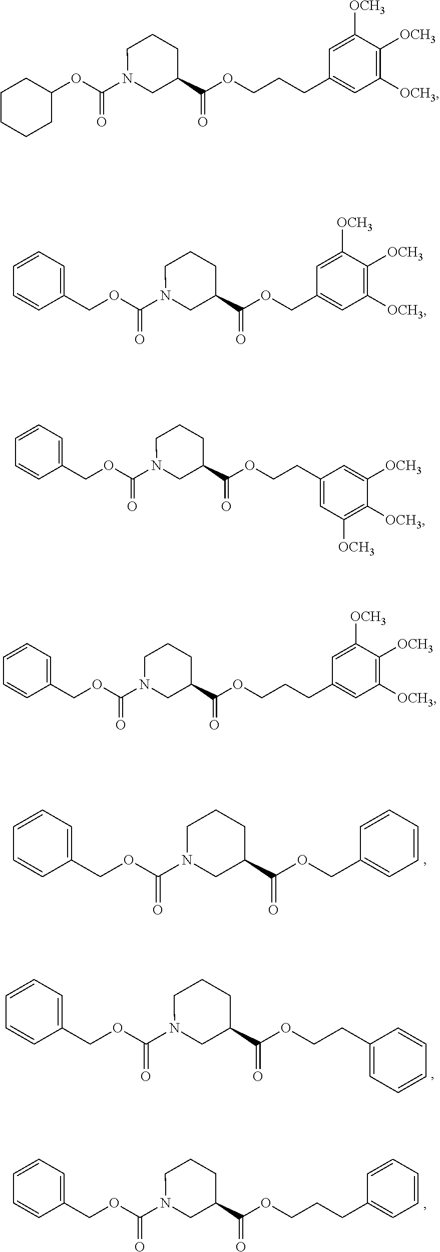

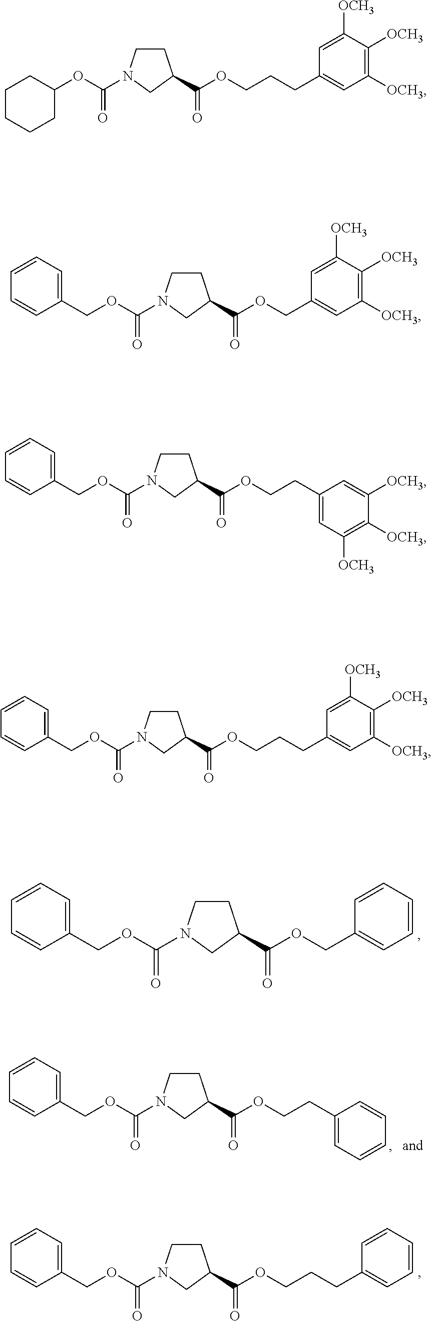

or a pharmaceutically acceptable salt thereof.

### C. Methods of Treating Muscle Wasting

In various aspects, the compounds and compositions disclosed herein are useful for treating, preventing, ameliorating, controlling or reducing the risk of muscle wasting in a subject in need thereof. Thus, in one aspect, disclosed are methods of treating muscle wasting in a subject, the method comprising administering to the subject an effective amount of at least one disclosed compound or a pharmaceutically acceptable salt thereof.

In one aspect, disclosed are methods of treating muscle wasting in a subject in need thereof, the method comprising administering to the subject an effective amount of a compound having a structure represented by a formula selected from:

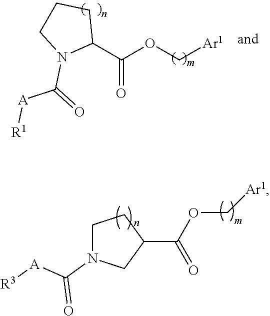

wherein n is 1 or 2; wherein m is 1, 2, or 3; wherein A is selected from —O— and —C(O)—; wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl); wherein q is 1, 2, or 3; wherein each of R^{20a} and R^{20b}, when present, is independently selected from hydrogen and C1-C4 alkyl; wherein Cy¹ is selected from cyclohexyl, a 6-membered heterocycloalkyl, and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; wherein R³ is selected from C1-C8 alkyl, (CH₂)_{q}Cy¹, and Cy¹; and wherein Ar¹ is selected from C6-aryl and pyridinyl, and is substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof, wherein the subject has not been diagnosed as having cancer prior to the administering step.

In one aspect, disclosed are methods of treating muscle wasting in a subject having a cancer, the method comprising administering to the subject an effective amount of a compound having a structure represented by a formula selected from:

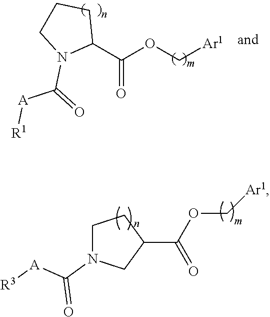

wherein n is 1 or 2; wherein m is 1, 2, or 3; wherein A is selected from —O— and —C(O)—; wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl); wherein q is 1, 2, or 3; wherein each of R^{20a} and R^{20b}, when present, is independently selected from hydrogen and C1-C4 alkyl; wherein Cy¹ is selected from cyclohexyl, a 6-membered heterocycloalkyl, and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; wherein R³ is selected from C1-C8 alkyl, (CH₂)_{q}Cy¹, and Cy¹; and wherein Ar¹ is selected from C6-aryl and pyridinyl, and is substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof, wherein the subject has experienced a weight loss of 5% or more over a time period of 12 months or less prior to the administering step.

In one aspect, disclosed are methods of treating muscle wasting in a subject having cancer, the method comprising administering to the subject an effective amount of a compound selected from:

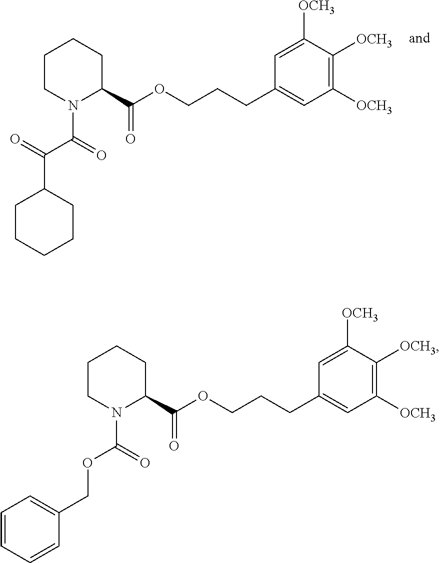 [SMILES: COc1cc(CCCOC(=O)[C@@H]2CCCCN2C(=O)C(=O)C2CCCCC2)cc(OC)c1OC ; COc1cc(CCCOC(=O)[C@@H]2CCCCN2C(=O)OCc2ccccc2)cc(OC)c1OC]

or a pharmaceutically acceptable salt thereof, wherein the cancer is selected from pancreatic cancer and gastric cancer, and wherein the subject has experienced a weight loss of 5% or more over a time period of 6 months or less prior to the administering step.

In various aspects, the disclosed compounds can be used in combination with one or more other drugs in the treatment, prevention, control, amelioration, or reduction of risk of muscle wasting for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly. Accordingly, the pharmaceutical compositions include those that contain one or more other active ingredients, in addition to a compound of the present invention.

In various aspects, n is 1. In a further aspect, n is 2.

In various aspects, A is —O—. In a further aspect, A is —C(O)—.

In various aspects, R¹ is selected from (CH₂)_{q}Cy¹, Cy¹, and C2-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl). In a further aspect, R¹ is C1-C8 acyclic alkyl substituted with 1, 2, or 3 groups independently selected from halogen, —CN, —NH₂, —NO₂, —OH, —C(O)NR^{20a}R^{20b}, —CO₂H, and —CO₂(C1-C4 alkyl). In a still further aspect, R¹ is selected from ethyl, n-propyl, isopropyl, n-butyl, isobutyl, sec-butyl, and t-butyl.

In various aspects, R¹ is selected from (CH₂)_{q}Cy¹ and Cy¹. In a further aspect, R¹ is Cy¹. In a still further aspect, R¹ is CH₂Cy¹.

In various aspects, R³ is selected from C2-C8 alkyl, (CH₂)_{q}Cy¹, and Cy¹. In a further aspect, R³ is selected from (CH₂)_{q}Cy¹ and Cy¹. In a still further aspect, R³ is Cy¹. In yet a further aspect, R³ is CH₂Cy¹.

In various aspects, Cy¹ is selected from cyclohexyl and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a further aspect, Cy¹ is an unsubstituted cyclohexyl. In a still further aspect, Cy¹ is an unsubstituted 6-membered monocyclic aryl.

In various aspects, Ar¹ is selected from C6-aryl and pyridinyl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In a further aspect, Ar¹ is selected from C6-aryl and pyridinyl, and is substituted with 0, 1, 2, or 3 C1-C4 alkoxy groups. In a still further aspect, Ar¹ is C6-aryl substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In yet a further aspect, Ar¹ is C6-aryl substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino. In an even further aspect, Ar¹ is C6-aryl substituted with 0, 1, 2, or 3 C1-C4 alkoxy groups.

In various aspects, the compound has a structure represented by a formula selected from:

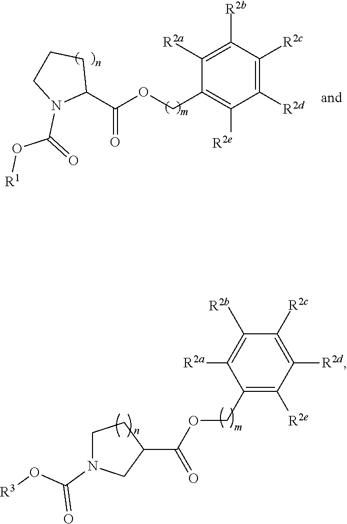

wherein n is 1 or 2; wherein m is 1, 2, or 3; wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl); wherein q, when present, is 1, 2, or 3; wherein each of R^{20a} and R^{20b}, when present, is independently selected from hydrogen and C1-C4 alkyl; wherein Cy¹ is selected from cyclohexyl and 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; and wherein each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; and wherein R³, when present, is selected from C1-C8 alkyl, (CH₂)_{q}Cy¹, and Cy¹, or a pharmaceutically acceptable salt thereof. In a further aspect, at least one of R^{2a}, R^{2b}, R^{2c}, R^{2d}, and R^{2e} is —OH, C1-C4 alkoxy, or C1-C4 haloalkoxy. In a still further aspect, when at least one of R^{2a}, R^{2b}, R^{2c}, R^{2d}, and R^{2e} is C1-C4 alkoxy and R¹ is C1-C8 acyclic alkyl, then R¹ is substituted with 1, 2, or 3 groups independently selected from halogen, —CN, —NH₂, —NO₂, —OH, —C(O)NR^{20a}R^{20b}, —CO₂H, and —CO₂(C1-C4 alkyl). In yet a further aspect, at least one of R^{2a}, R^{2b}, R^{2c}, R^{2d}, and R^{2e} is C1-C4 alkoxy or C1-C4 haloalkoxy. In an even further aspect, each of R^{2b}, R^{2c}, and R^{2d} is methoxy. In a still further aspect, the compound has a structure represented by a formula selected from:

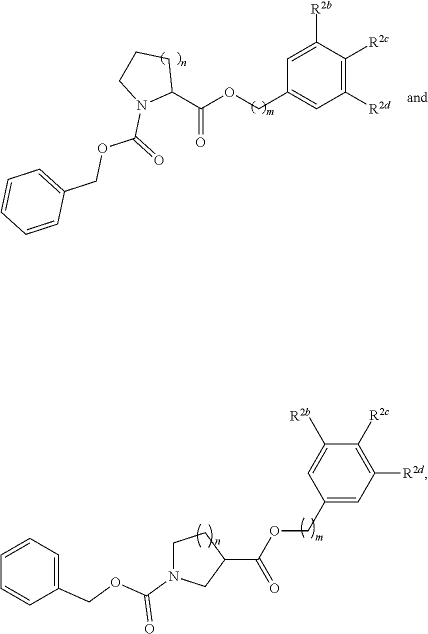

or a pharmaceutically acceptable salt thereof.

In various aspects, the compound has a structure represented by a formula selected from:

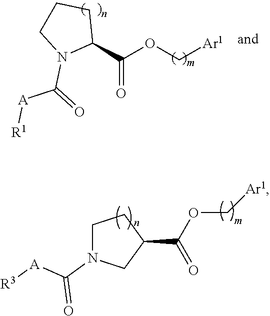

or a pharmaceutically acceptable salt thereof.

In various aspects, the compound has a structure represented by a formula:

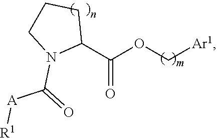

or a pharmaceutically acceptable salt thereof.

In various aspects, the compound has a structure represented by a formula:

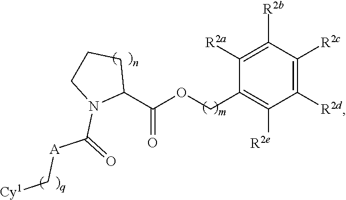

wherein each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof.

In various aspects, the compound has a structure represented by a formula:

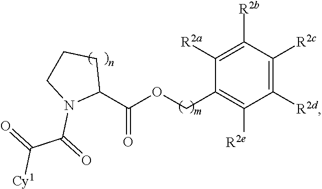

or a pharmaceutically acceptable salt thereof.

In various aspects, the compound has a structure selected from:

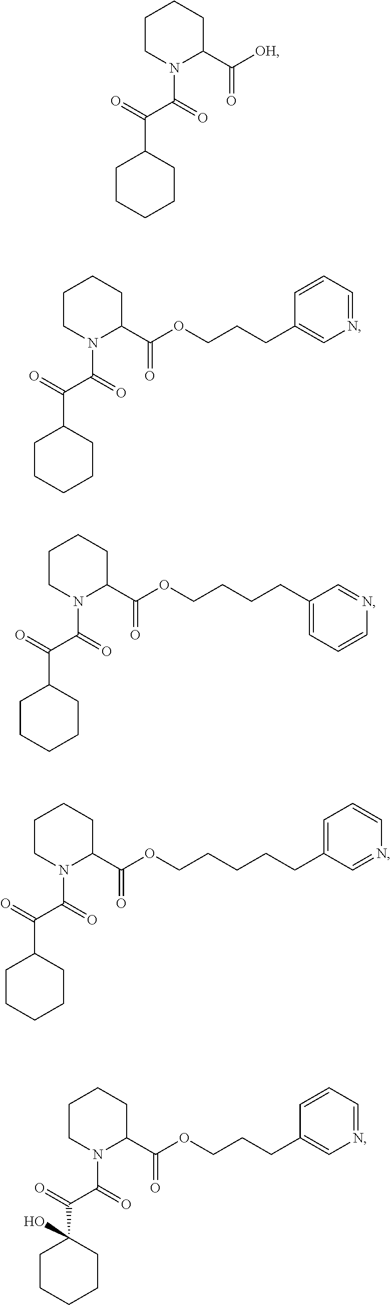

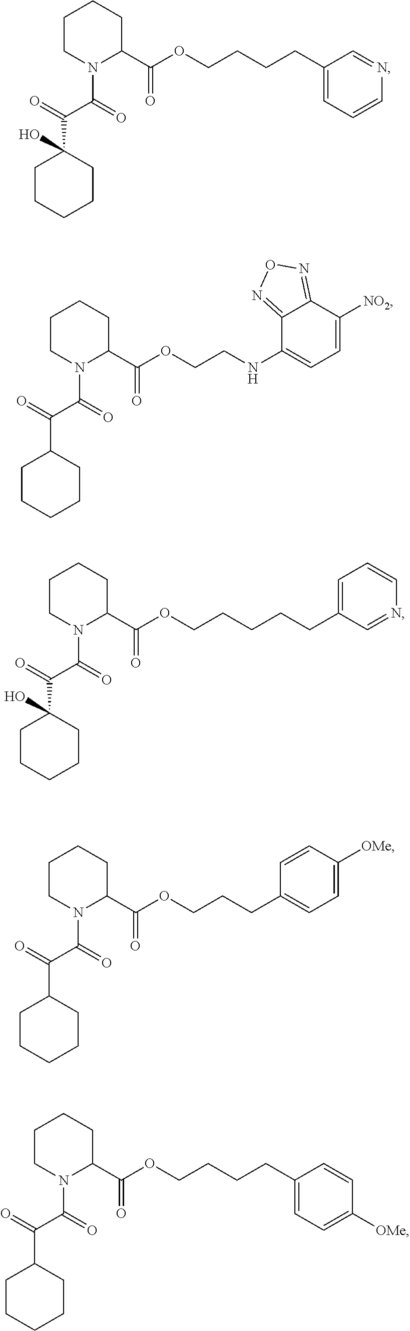

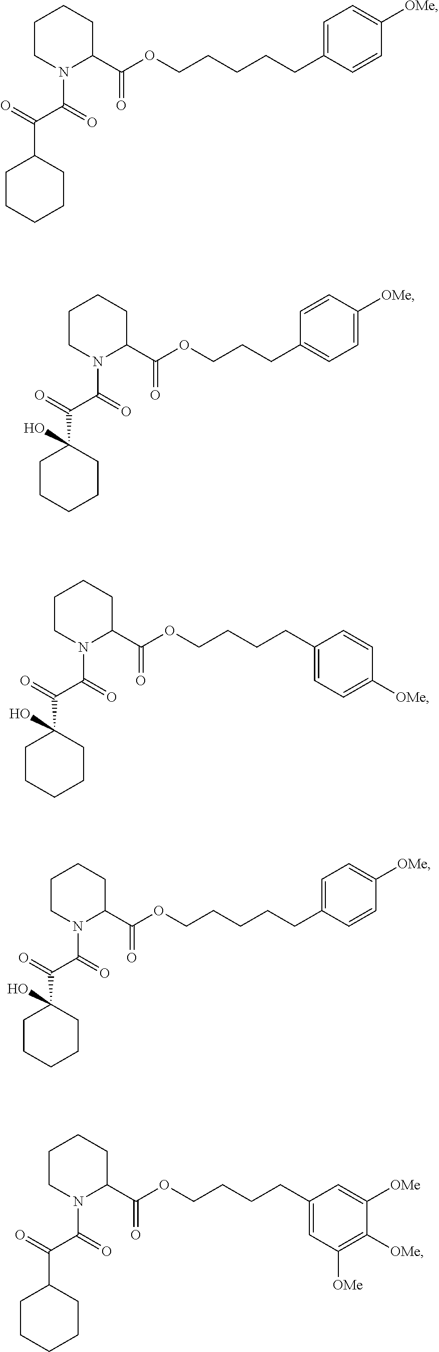

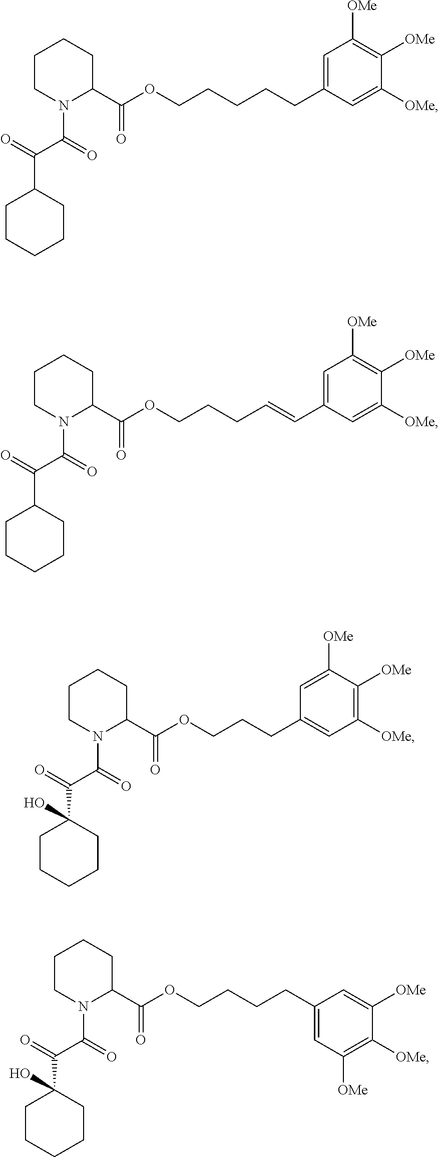

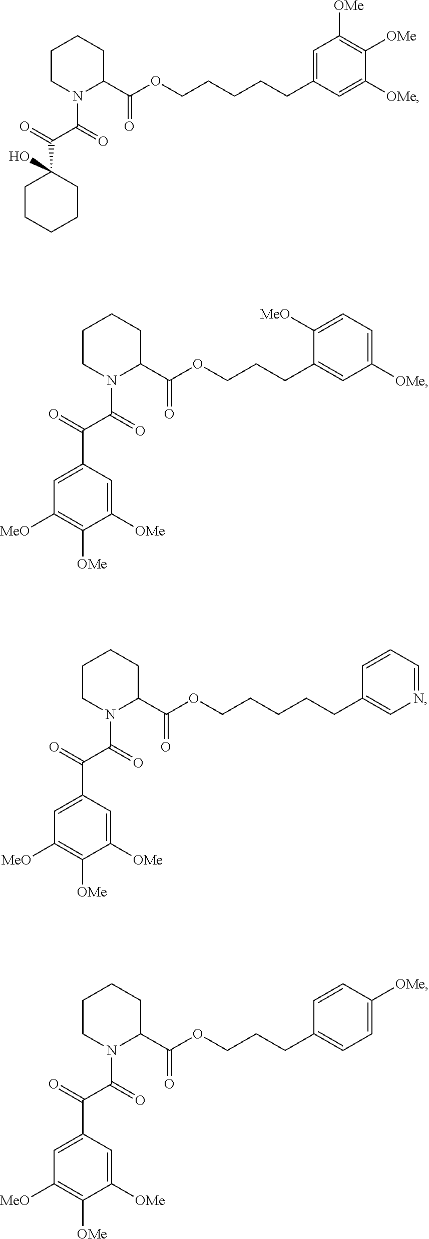

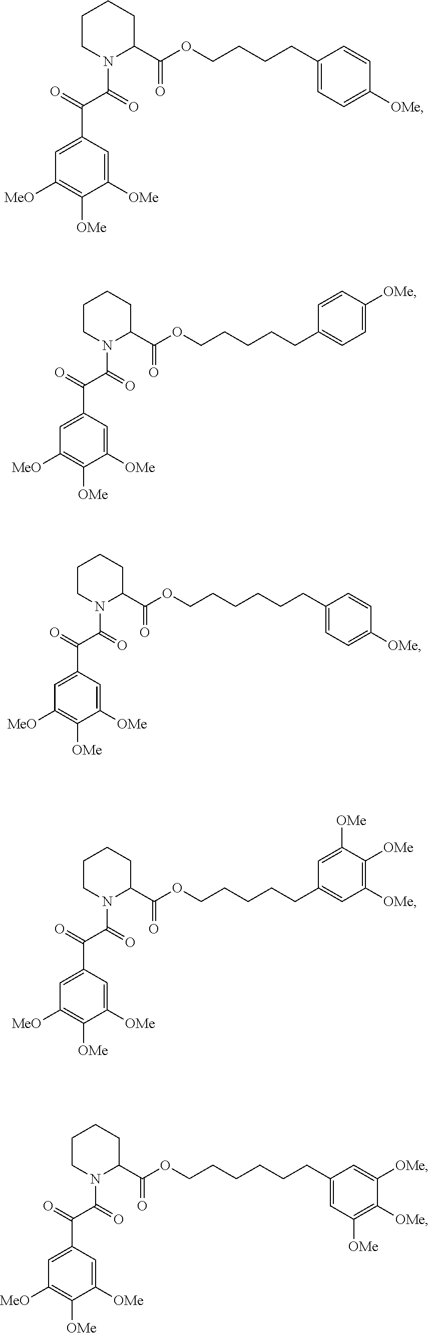 [SMILES: COc1ccc(CCCCOC(=O)C2CCCCN2C(=O)C(=O)c2cc(OC)c(OC)c(OC)c2)cc1 ; COc1ccc(CCCCCOC(=O)C2CCCCN2C(=O)C(=O)c2cc(OC)c(OC)c(OC)c2)cc1 ; COc1ccc(CCCCCCOC(=O)C2CCCCN2C(=O)C(=O)c2cc(OC)c(OC)c(OC)c2)cc1 ; COc1cc(CCCCCOC(=O)C2CCCCN2C(=O)C(=O)c2cc(OC)c(OC)c(OC)c2)cc(OC)c1OC ; COc1cc(CCCCCCOC(=O)C2CCCCN2C(=O)C(=O)c2cc(OC)c(OC)c(OC)c2)cc(OC)c1OC]

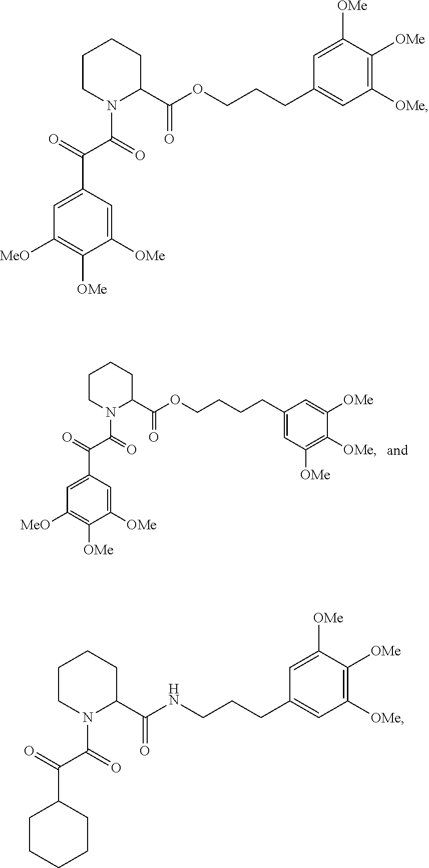 [SMILES: COc1cc(CCCOC(=O)C2CCCCN2C(=O)C(=O)c2cc(OC)c(OC)c(OC)c2)cc(OC)c1OC ; COc1cc(CCCCOC(=O)C2CCCCN2C(=O)C(=O)c2cc(OC)c(OC)c(OC)c2)cc(OC)c1OC ; "and" ; COc1cc(CCCNC(=O)C2CCCCN2C(=O)C(=O)C2CCCCC2)cc(OC)c1OC]

or a pharmaceutically acceptable salt thereof.

In various aspects, the compound has a structure selected from:

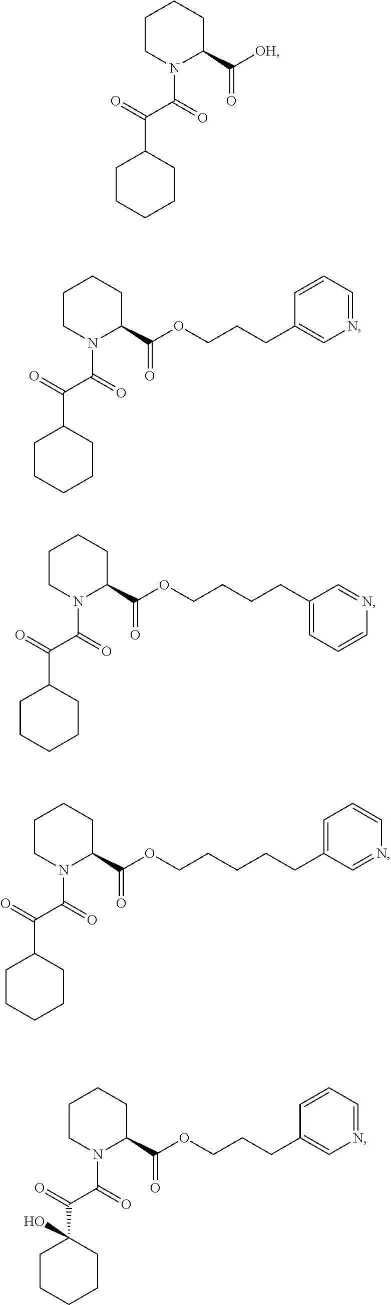

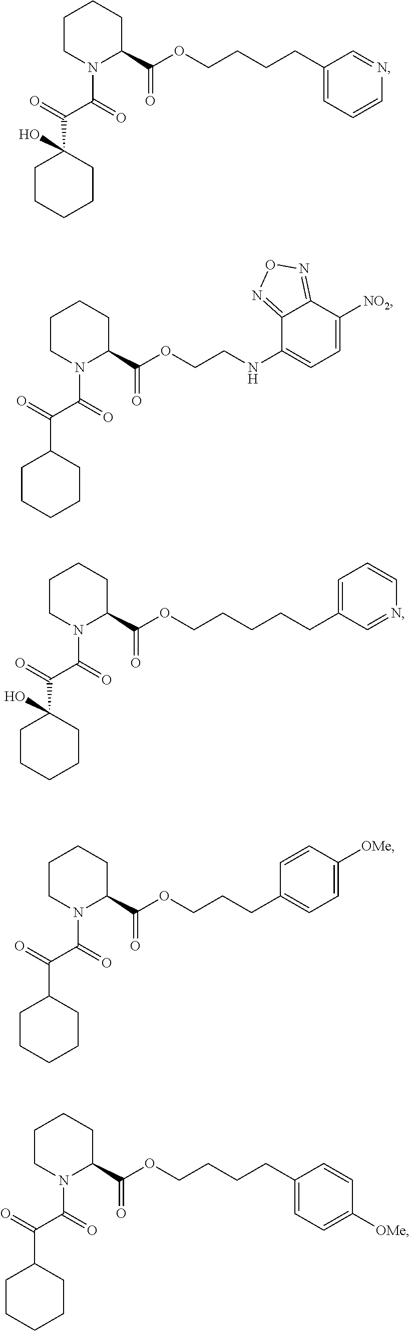

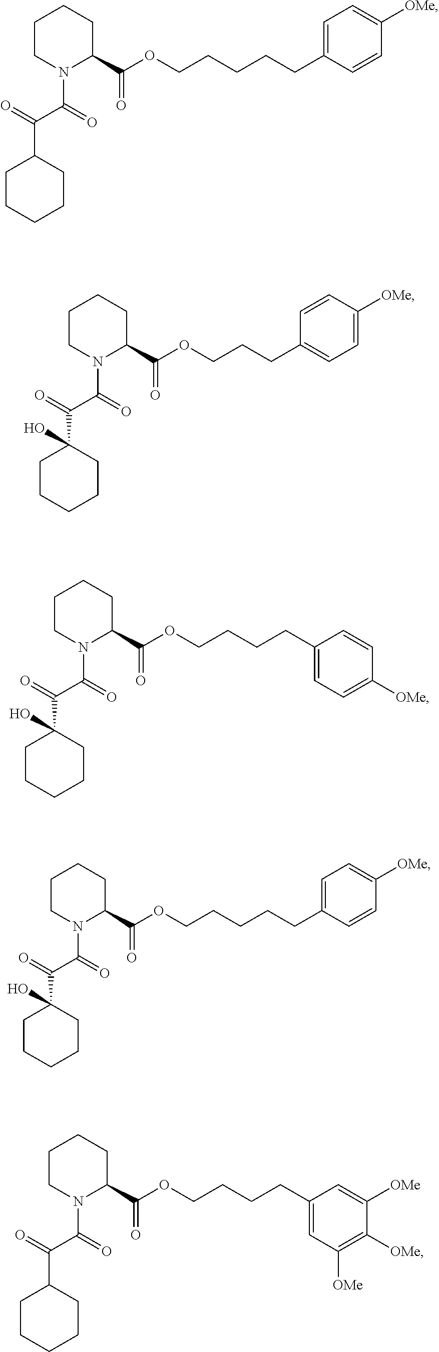

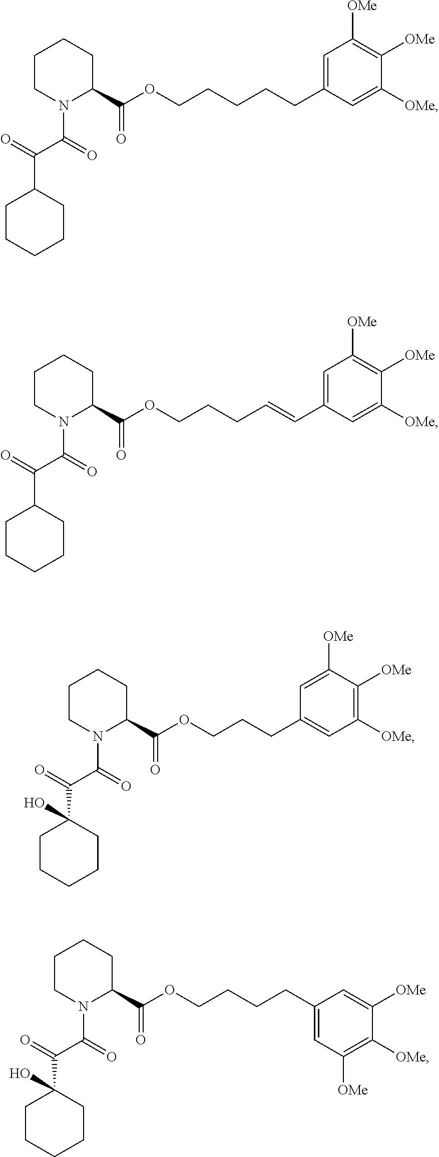

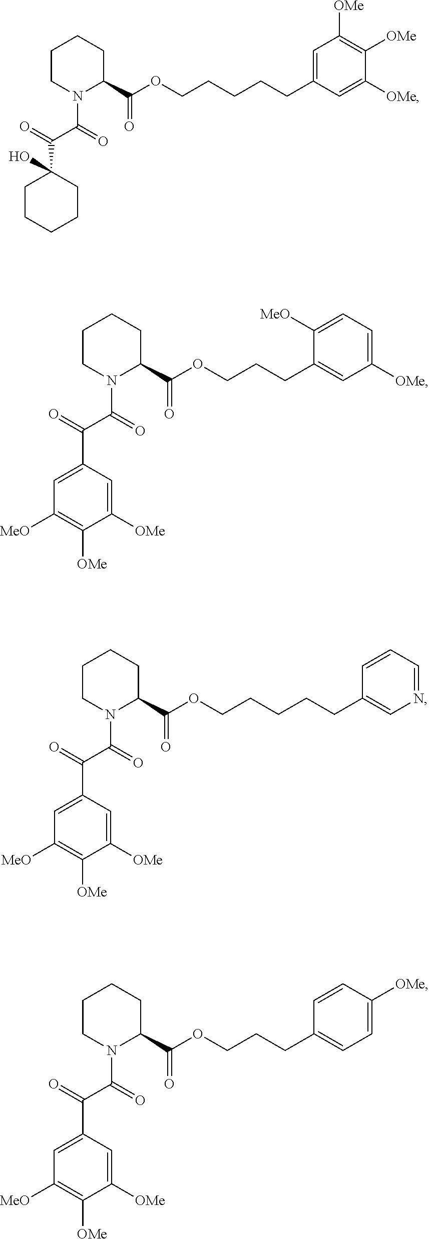

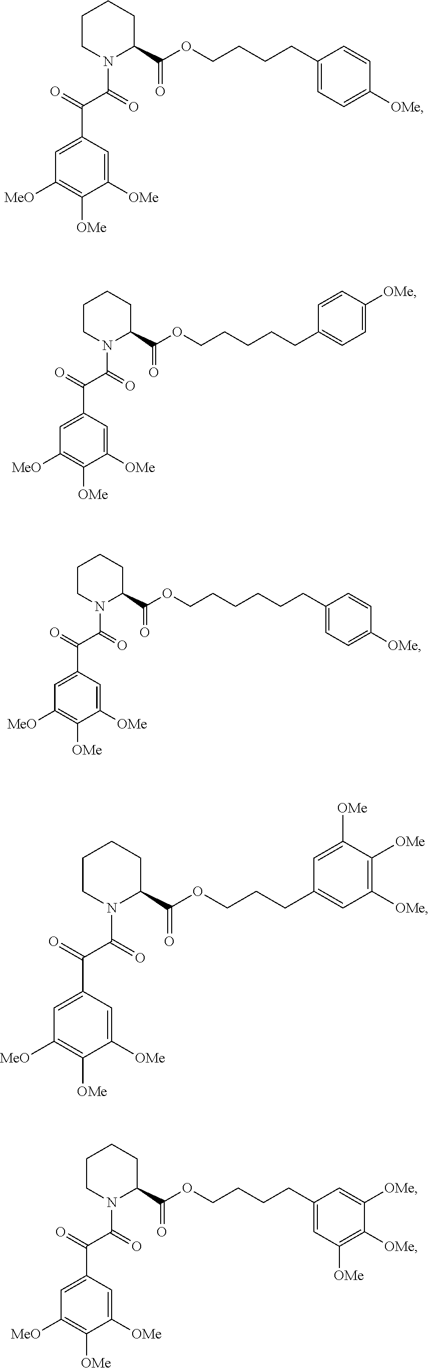 [SMILES: COc1ccc(CCCCOC(=O)[C@@H]2CCCCN2C(=O)C(=O)c2cc(OC)c(OC)c(OC)c2)cc1 ; COc1ccc(CCCCCOC(=O)[C@@H]2CCCCN2C(=O)C(=O)c2cc(OC)c(OC)c(OC)c2)cc1 ; COc1ccc(CCCCCCOC(=O)[C@@H]2CCCCN2C(=O)C(=O)c2cc(OC)c(OC)c(OC)c2)cc1 ; COc1cc(CCCOC(=O)[C@@H]2CCCCN2C(=O)C(=O)c2cc(OC)c(OC)c(OC)c2)cc(OC)c1OC ; COc1cc(CCCCOC(=O)[C@@H]2CCCCN2C(=O)C(=O)c2cc(OC)c(OC)c(OC)c2)cc(OC)c1OC]

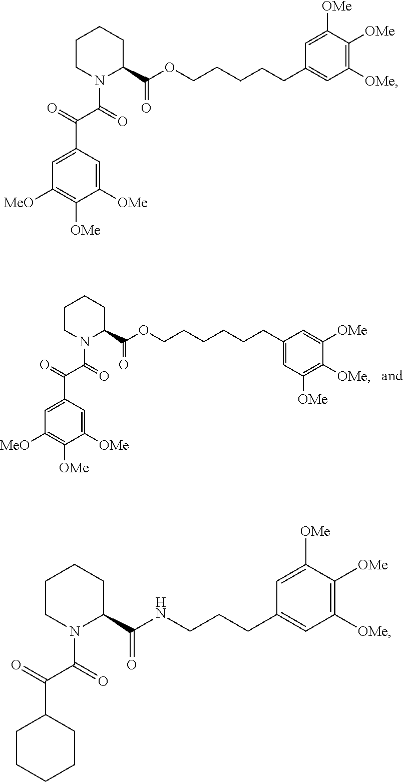 [SMILES: COc1cc(CCCCCOC(=O)[C@@H]2CCCCN2C(=O)C(=O)c2cc(OC)c(OC)c(OC)c2)cc(OC)c1OC ; COc1cc(CCCCCCOC(=O)[C@@H]2CCCCN2C(=O)C(=O)c2cc(OC)c(OC)c(OC)c2)cc(OC)c1OC ; "and" ; COc1cc(CCCNC(=O)[C@@H]2CCCCN2C(=O)C(=O)C2CCCCC2)cc(OC)c1OC]

or a pharmaceutically acceptable salt thereof.

In various aspects, the compound has a structure:

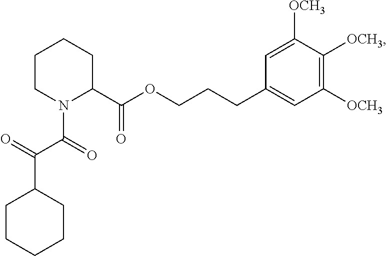 [SMILES: COc1cc(CCCOC(=O)C2CCCCN2C(=O)C(=O)C2CCCCC2)cc(OC)c1OC]

or a pharmaceutically acceptable salt thereof.

In various aspects, the compound has a structure:

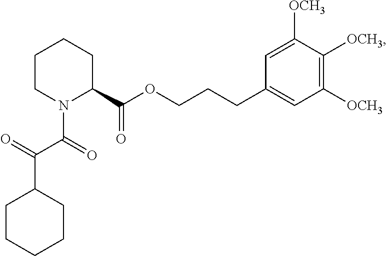 [SMILES: COc1cc(CCCOC(=O)[C@@H]2CCCCN2C(=O)C(=O)C2CCCCC2)cc(OC)c1OC]

or a pharmaceutically acceptable salt thereof.

In various aspects, the compound has a structure represented by a formula:

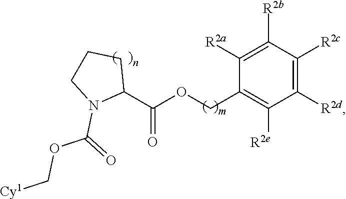

or a pharmaceutically acceptable salt thereof.

In various aspects, the compound has a structure selected from:

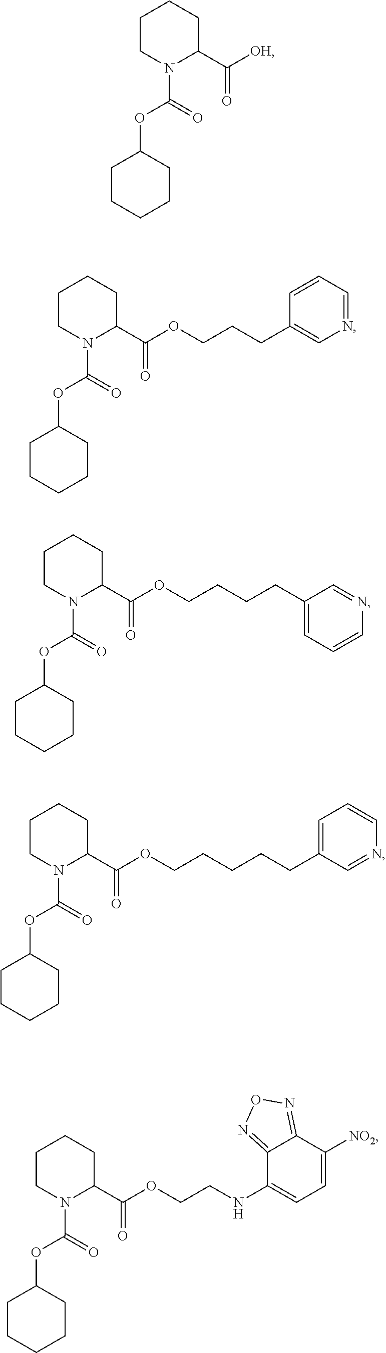

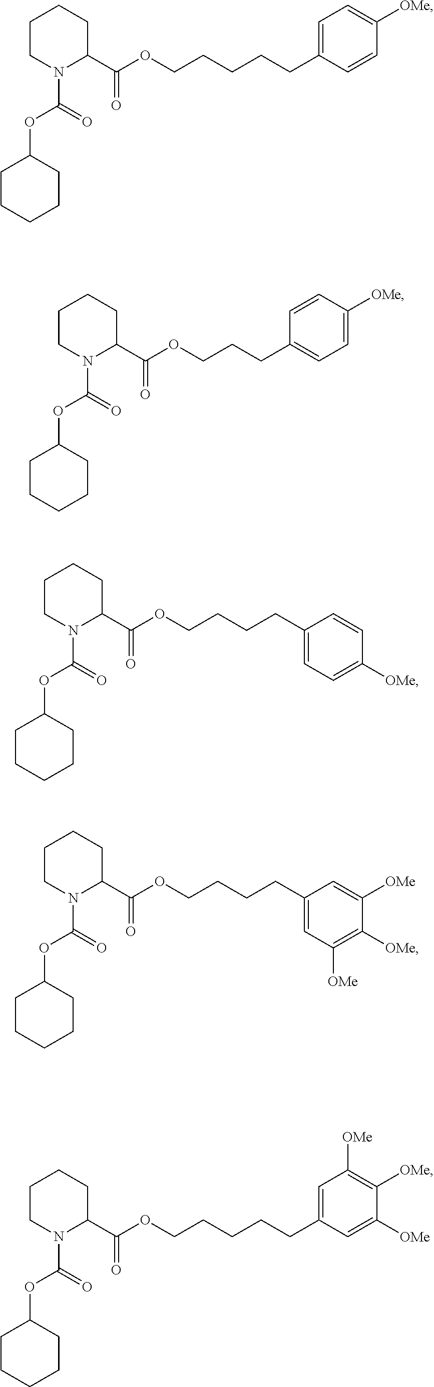 [SMILES: COc1ccc(CCCCCOC(=O)C2CCCCN2C(=O)OC2CCCCC2)cc1 ; COc1ccc(CCCOC(=O)C2CCCCN2C(=O)OC2CCCCC2)cc1 ; COc1ccc(CCCCOC(=O)C2CCCCN2C(=O)OC2CCCCC2)cc1 ; COc1cc(CCCCOC(=O)C2CCCCN2C(=O)OC2CCCCC2)cc(OC)c1OC ; COc1cc(CCCCCOC(=O)C2CCCCN2C(=O)OC2CCCCC2)cc(OC)c1OC]

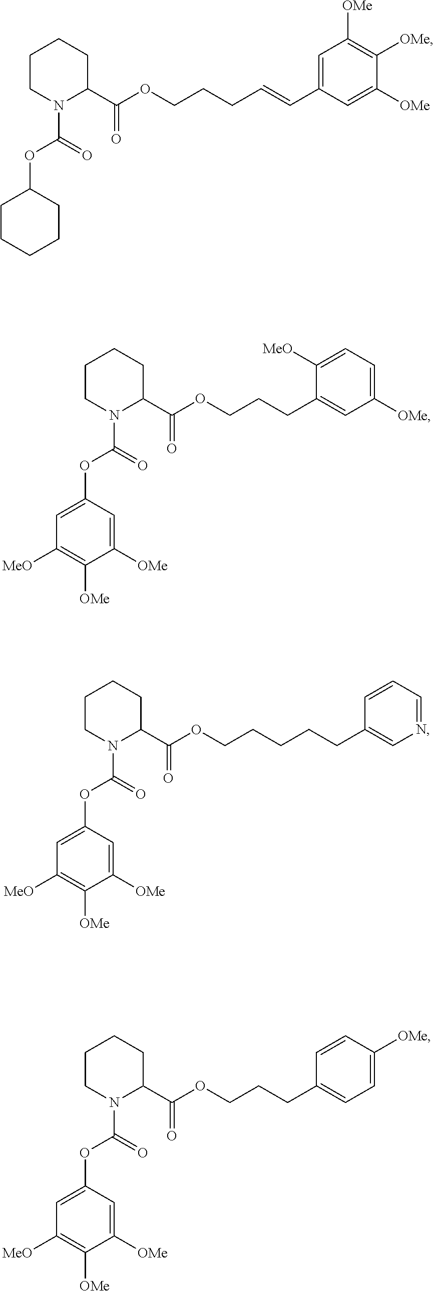 [SMILES: COc1cc(/C=C/CCCOC(=O)C2CCCCN2C(=O)OC2CCCCC2)cc(OC)c1OC ; COc1ccc(OC)c(CCCOC(=O)C2CCCCN2C(=O)Oc2cc(OC)c(OC)c(OC)c2)c1 ; COc1cc(OC(=O)N2CCCCC2C(=O)OCCCCCc2cccnc2)cc(OC)c1OC ; COc1ccc(CCCOC(=O)C2CCCCN2C(=O)Oc2cc(OC)c(OC)c(OC)c2)cc1]

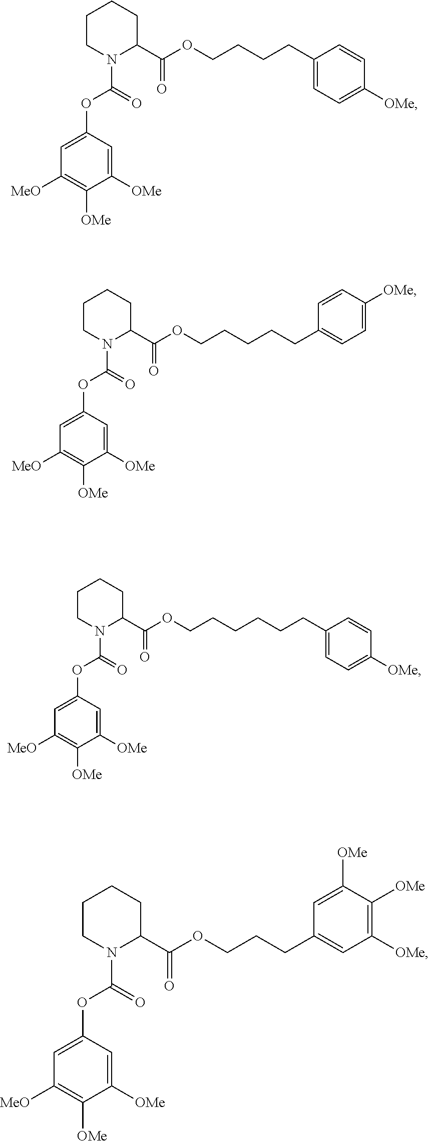 [SMILES: COc1ccc(CCCCOC(=O)C2CCCCN2C(=O)Oc2cc(OC)c(OC)c(OC)c2)cc1 ; COc1ccc(CCCCCOC(=O)C2CCCCN2C(=O)Oc2cc(OC)c(OC)c(OC)c2)cc1 ; COc1ccc(CCCCCCOC(=O)C2CCCCN2C(=O)Oc2cc(OC)c(OC)c(OC)c2)cc1 ; COc1cc(CCCOC(=O)C2CCCCN2C(=O)Oc2cc(OC)c(OC)c(OC)c2)cc(OC)c1OC]

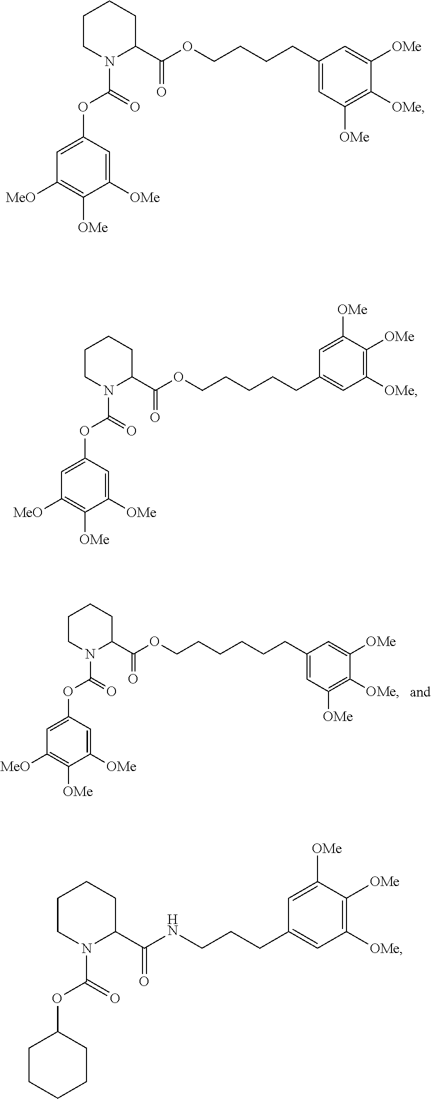 [SMILES: COc1cc(CCCCOC(=O)C2CCCCN2C(=O)Oc2cc(OC)c(OC)c(OC)c2)cc(OC)c1OC ; COc1cc(CCCCCOC(=O)C2CCCCN2C(=O)Oc2cc(OC)c(OC)c(OC)c2)cc(OC)c1OC ; COc1cc(CCCCCCOC(=O)C2CCCCN2C(=O)Oc2cc(OC)c(OC)c(OC)c2)cc(OC)c1OC ; "and" ; COc1cc(CCCNC(=O)C2CCCCN2C(=O)OC2CCCCC2)cc(OC)c1OC]

or a pharmaceutically acceptable salt thereof.

In various aspects, the compound has a structure selected from:

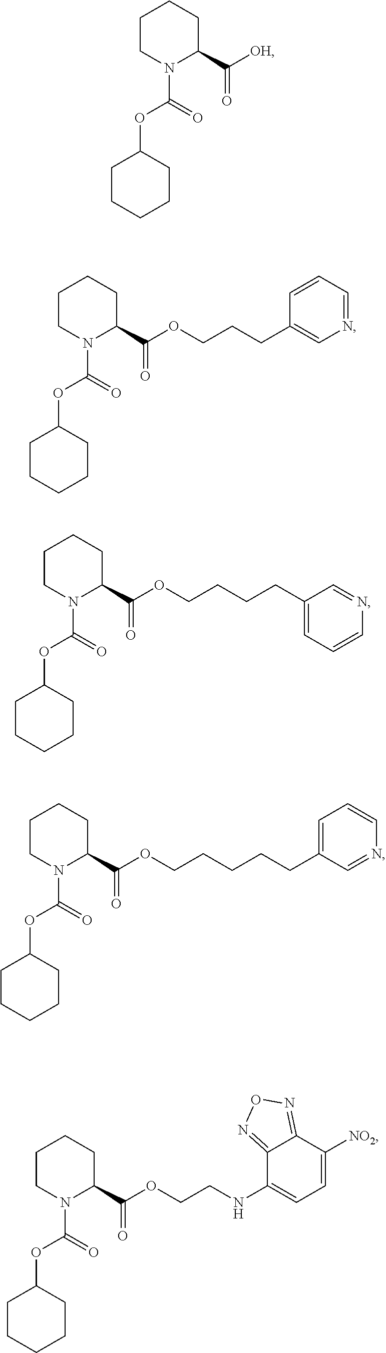

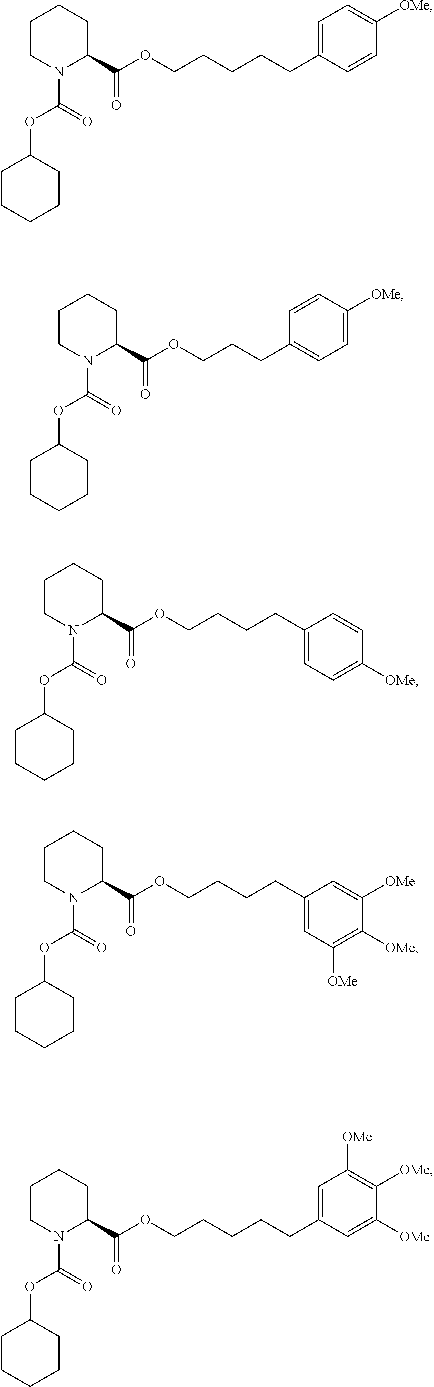 [SMILES: COc1ccc(CCCCCOC(=O)[C@@H]2CCCCN2C(=O)OC2CCCCC2)cc1 ; COc1ccc(CCCOC(=O)[C@@H]2CCCCN2C(=O)OC2CCCCC2)cc1 ; COc1ccc(CCCCOC(=O)[C@@H]2CCCCN2C(=O)OC2CCCCC2)cc1 ; COc1cc(CCCCOC(=O)[C@@H]2CCCCN2C(=O)OC2CCCCC2)cc(OC)c1OC ; COc1cc(CCCCCOC(=O)[C@@H]2CCCCN2C(=O)OC2CCCCC2)cc(OC)c1OC]

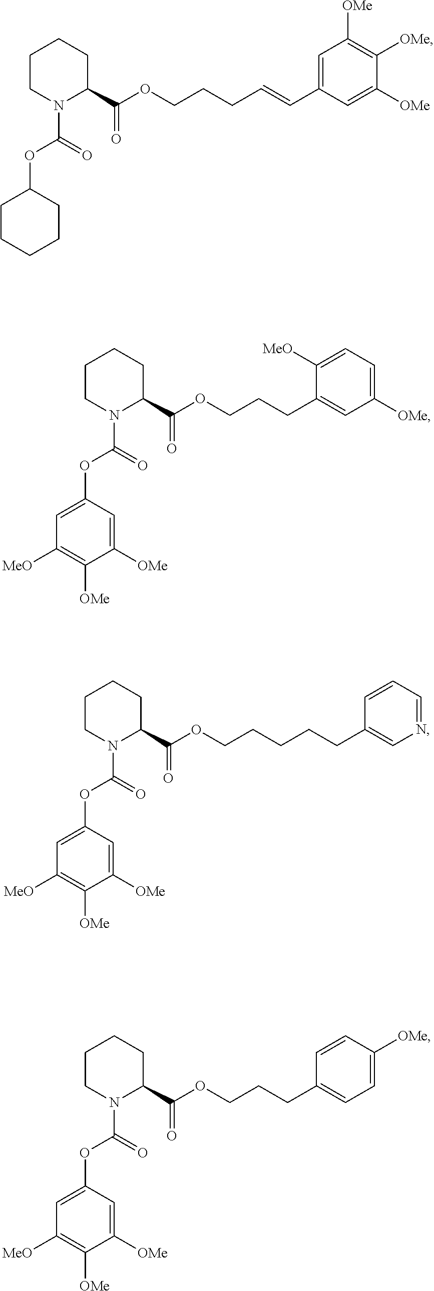 [SMILES: COc1cc(/C=C/CCCOC(=O)[C@@H]2CCCCN2C(=O)OC2CCCCC2)cc(OC)c1OC ; COc1ccc(OC)c(CCCOC(=O)[C@@H]2CCCCN2C(=O)Oc2cc(OC)c(OC)c(OC)c2)c1 ; COc1cc(OC(=O)N2CCCC[C@H]2C(=O)OCCCCCc2cccnc2)cc(OC)c1OC ; COc1ccc(CCCOC(=O)[C@@H]2CCCCN2C(=O)Oc2cc(OC)c(OC)c(OC)c2)cc1]

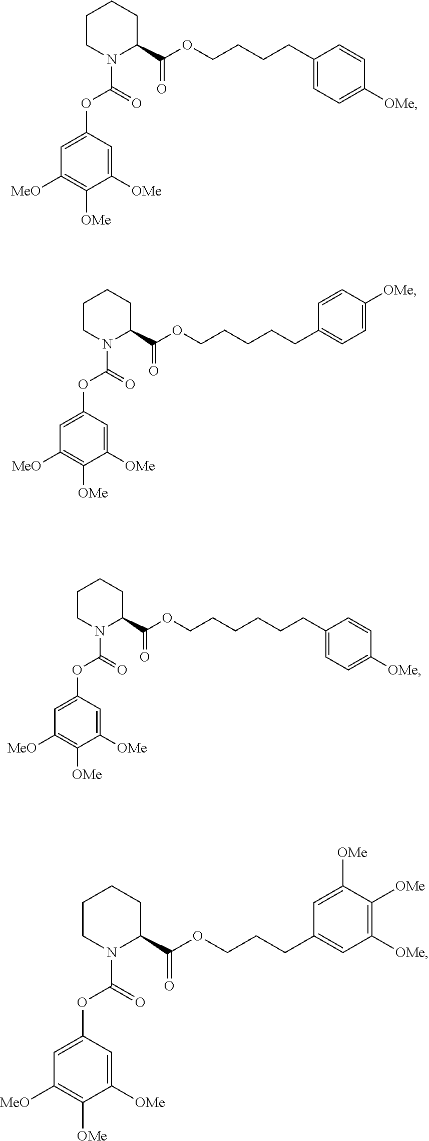 [SMILES: COc1ccc(CCCCOC(=O)[C@@H]2CCCCN2C(=O)Oc2cc(OC)c(OC)c(OC)c2)cc1 ; COc1ccc(CCCCCOC(=O)[C@@H]2CCCCN2C(=O)Oc2cc(OC)c(OC)c(OC)c2)cc1 ; COc1ccc(CCCCCCOC(=O)[C@@H]2CCCCN2C(=O)Oc2cc(OC)c(OC)c(OC)c2)cc1 ; COc1cc(CCCOC(=O)[C@@H]2CCCCN2C(=O)Oc2cc(OC)c(OC)c(OC)c2)cc(OC)c1OC]

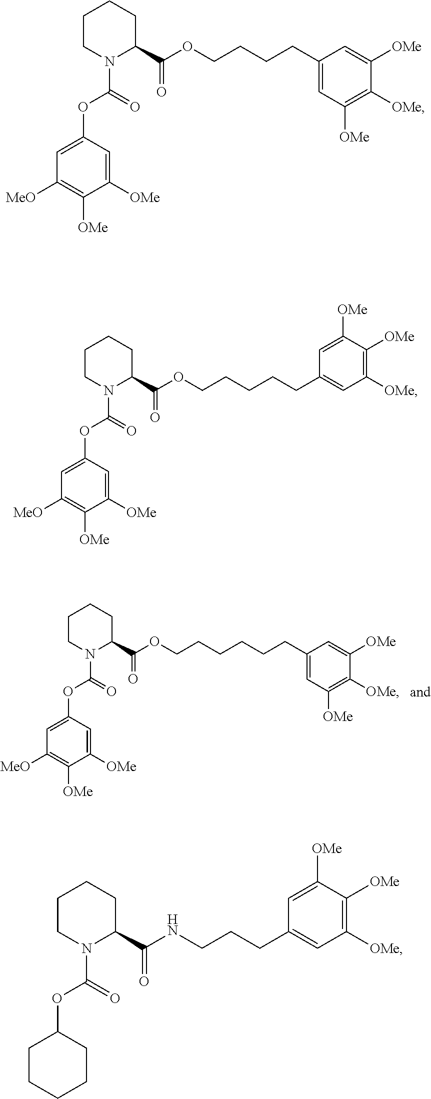 [SMILES: COc1cc(CCCCOC(=O)[C@@H]2CCCCN2C(=O)Oc2cc(OC)c(OC)c(OC)c2)cc(OC)c1OC ; COc1cc(CCCCCOC(=O)[C@@H]2CCCCN2C(=O)Oc2cc(OC)c(OC)c(OC)c2)cc(OC)c1OC ; COc1cc(CCCCCCOC(=O)[C@@H]2CCCCN2C(=O)Oc2cc(OC)c(OC)c(OC)c2)cc(OC)c1OC ; "and" ; COc1cc(CCCNC(=O)[C@@H]2CCCCN2C(=O)OC2CCCCC2)cc(OC)c1OC]

or a pharmaceutically acceptable salt thereof.

In various aspects, the compound is selected from:

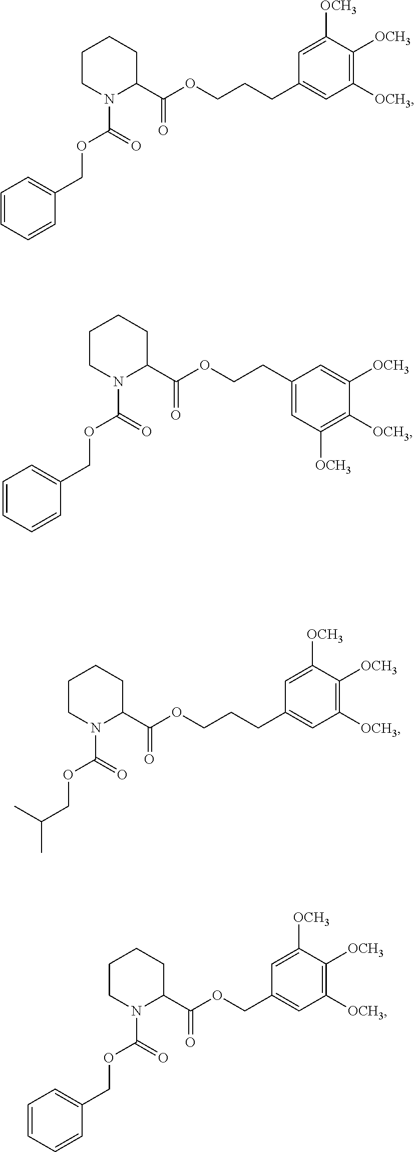 [SMILES: COc1cc(CCCOC(=O)C2CCCCN2C(=O)OCc2ccccc2)cc(OC)c1OC ; COc1cc(CCOC(=O)C2CCCCN2C(=O)OCc2ccccc2)cc(OC)c1OC ; COc1cc(CCCOC(=O)C2CCCCN2C(=O)OCC(C)C)cc(OC)c1OC ; COc1cc(COC(=O)C2CCCCN2C(=O)OCc2ccccc2)cc(OC)c1OC]

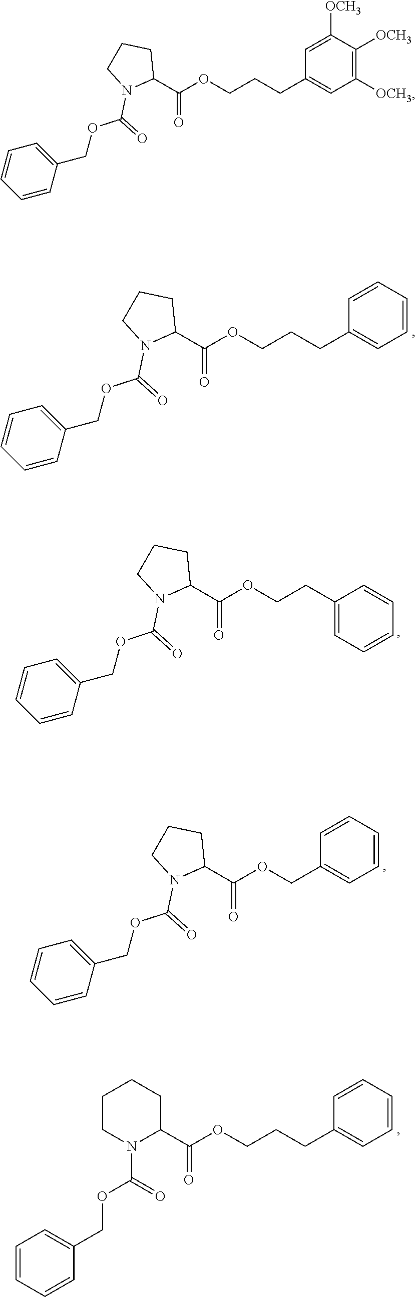 [SMILES: COc1cc(CCCOC(=O)C2CCCN2C(=O)OCc2ccccc2)cc(OC)c1OC ; O=C(OCCCc1ccccc1)C1CCCN1C(=O)OCc1ccccc1 ; O=C(OCCc1ccccc1)C1CCCN1C(=O)OCc1ccccc1 ; O=C(OCc1ccccc1)C1CCCN1C(=O)OCc1ccccc1 ; O=C(OCCCc1ccccc1)C1CCCCN1C(=O)OCc1ccccc1]

or a pharmaceutically acceptable salt thereof.

In various aspects, the compound is selected from:

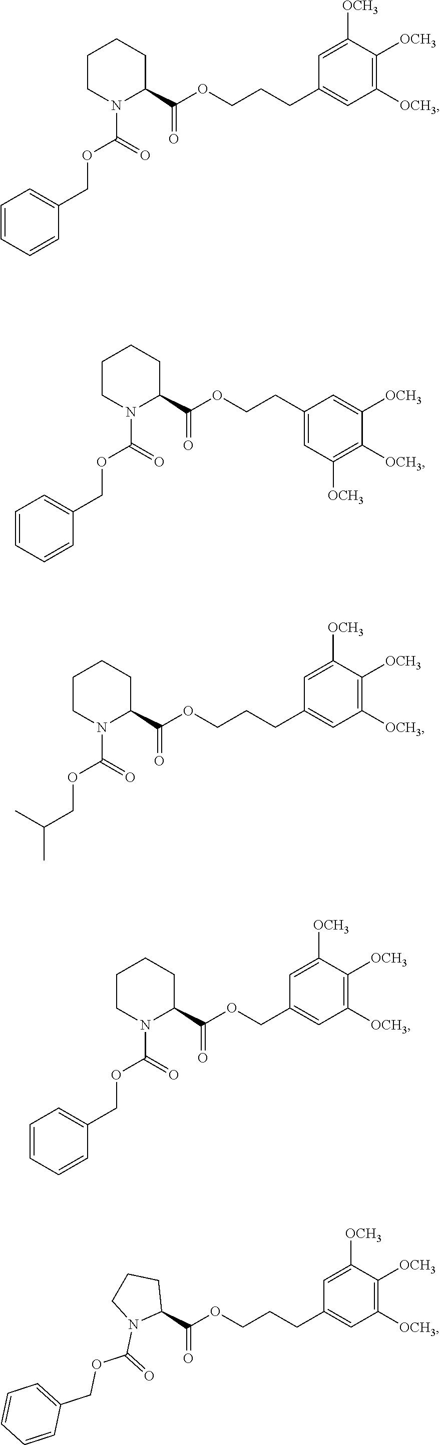 [SMILES: COc1cc(CCCOC(=O)[C@@H]2CCCCN2C(=O)OCc2ccccc2)cc(OC)c1OC ; COc1cc(CCOC(=O)[C@@H]2CCCCN2C(=O)OCc2ccccc2)cc(OC)c1OC ; COc1cc(CCCOC(=O)[C@@H]2CCCCN2C(=O)OCC(C)C)cc(OC)c1OC ; COc1cc(COC(=O)[C@@H]2CCCCN2C(=O)OCc2ccccc2)cc(OC)c1OC ; COc1cc(CCCOC(=O)[C@@H]2CCCN2C(=O)OCc2ccccc2)cc(OC)c1OC]

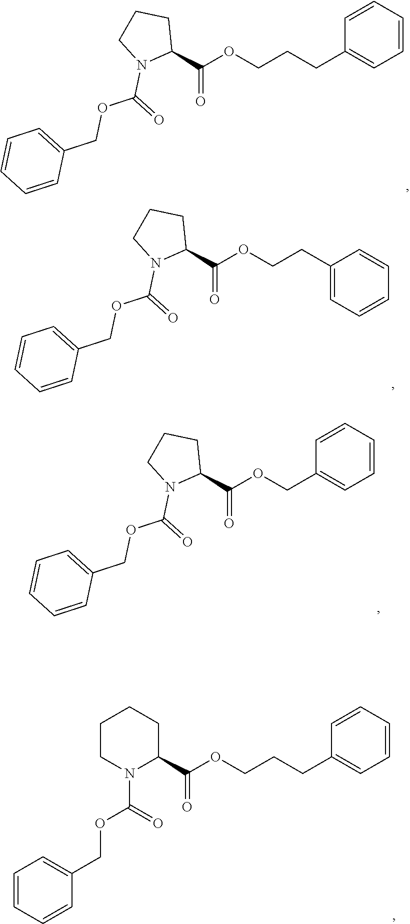 [SMILES: O=C(OCCCc1ccccc1)[C@@H]1CCCN1C(=O)OCc1ccccc1 ; O=C(OCCc1ccccc1)[C@@H]1CCCN1C(=O)OCc1ccccc1 ; O=C(OCc1ccccc1)[C@@H]1CCCN1C(=O)OCc1ccccc1 ; O=C(OCCCc1ccccc1)[C@@H]1CCCCN1C(=O)OCc1ccccc1]

or a pharmaceutically acceptable salt thereof.

In various aspects, the compound is:

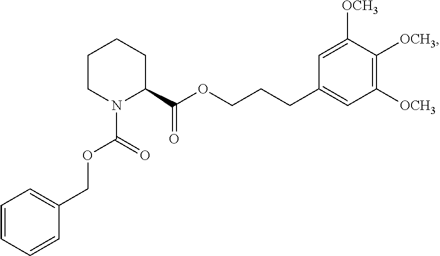 [SMILES: COc1cc(CCCOC(=O)[C@@H]2CCCCN2C(=O)OCc2ccccc2)cc(OC)c1OC]

or a pharmaceutically acceptable salt thereof.

In various aspects, the compound has a structure:

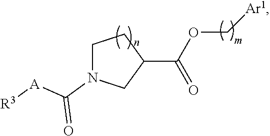

or a pharmaceutically acceptable salt thereof.

In various aspects, the compound has a structure represented by a formula:

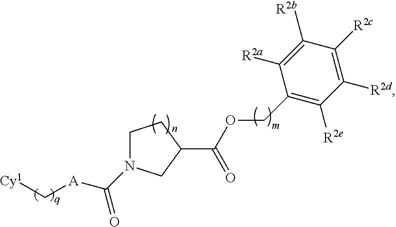

wherein each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof.

In various aspects, the compound has a structure represented by a formula:

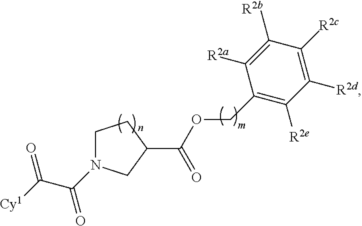

or a pharmaceutically acceptable salt thereof.

In various aspects, the compound has a structure selected from:

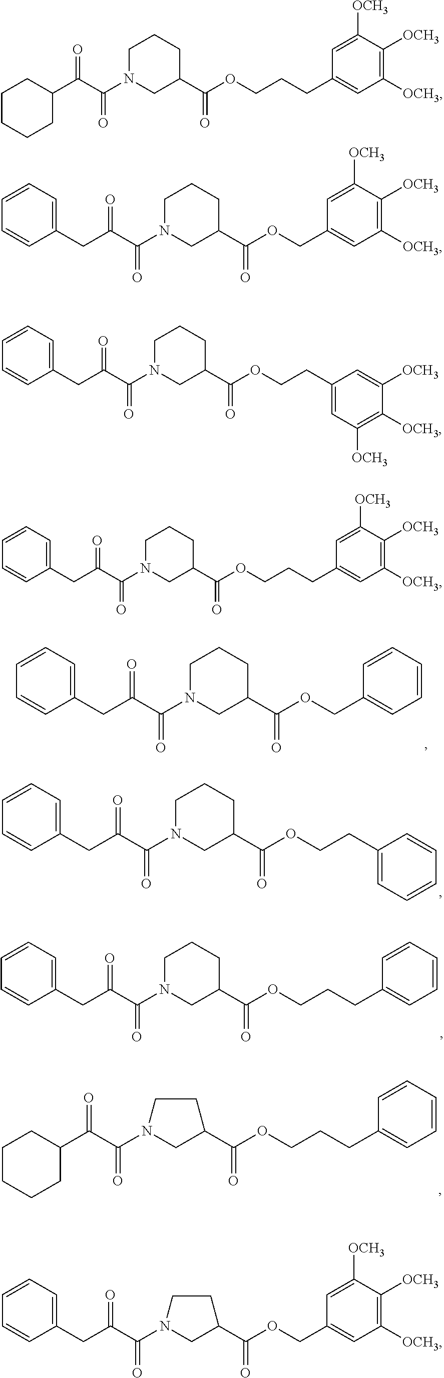

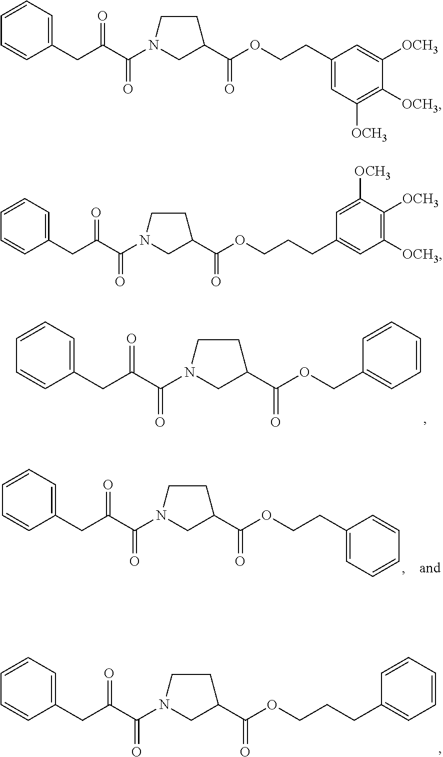

or a pharmaceutically acceptable salt thereof.

In various aspects, the compound has a structure selected from:

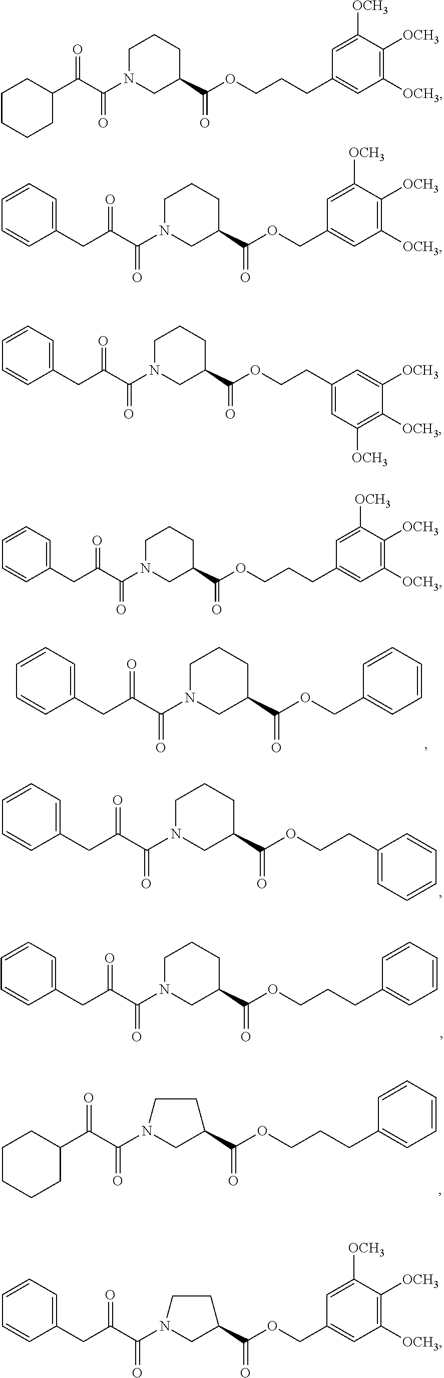

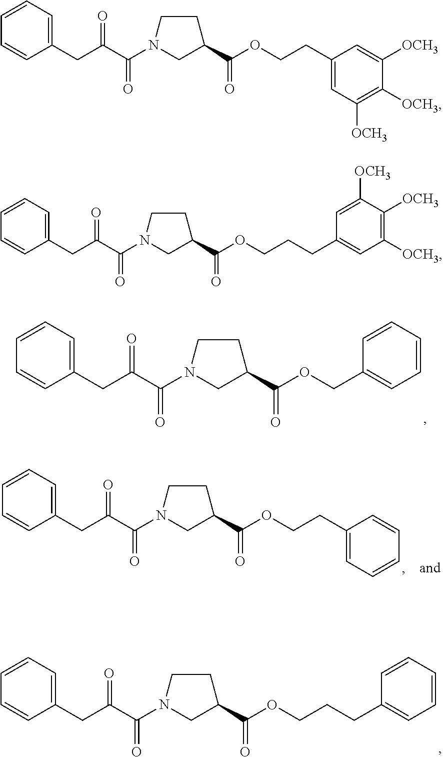

or a pharmaceutically acceptable salt thereof.

In various aspects, the compound has a structure represented by a formula:

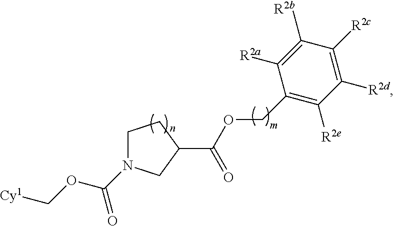

or a pharmaceutically acceptable salt thereof.

In various aspects, the compound has a structure selected from:

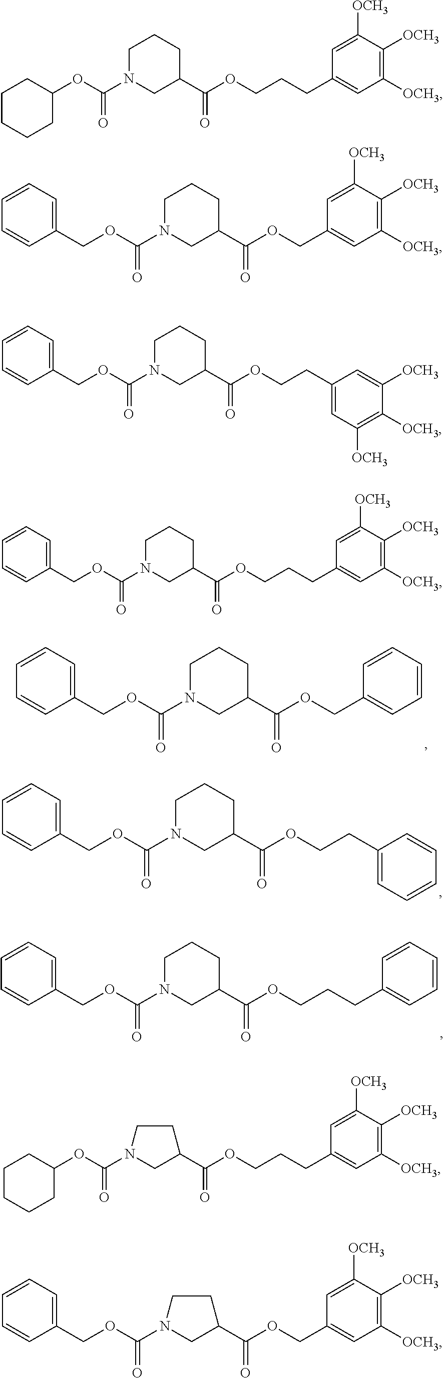

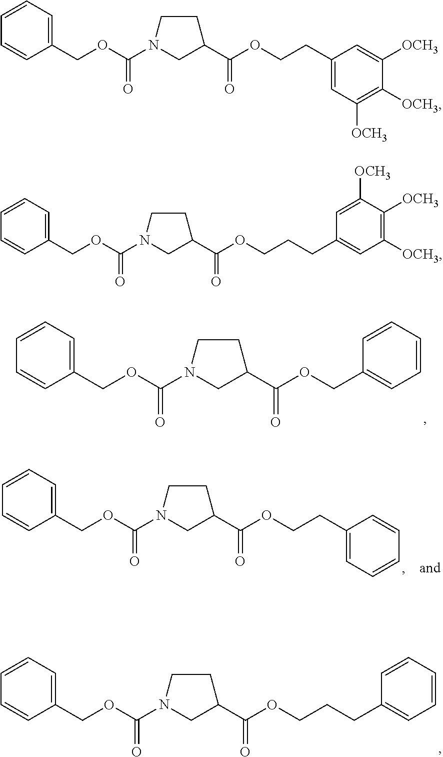

or a pharmaceutically acceptable salt thereof.

In various aspects, the compound has a structure selected from:

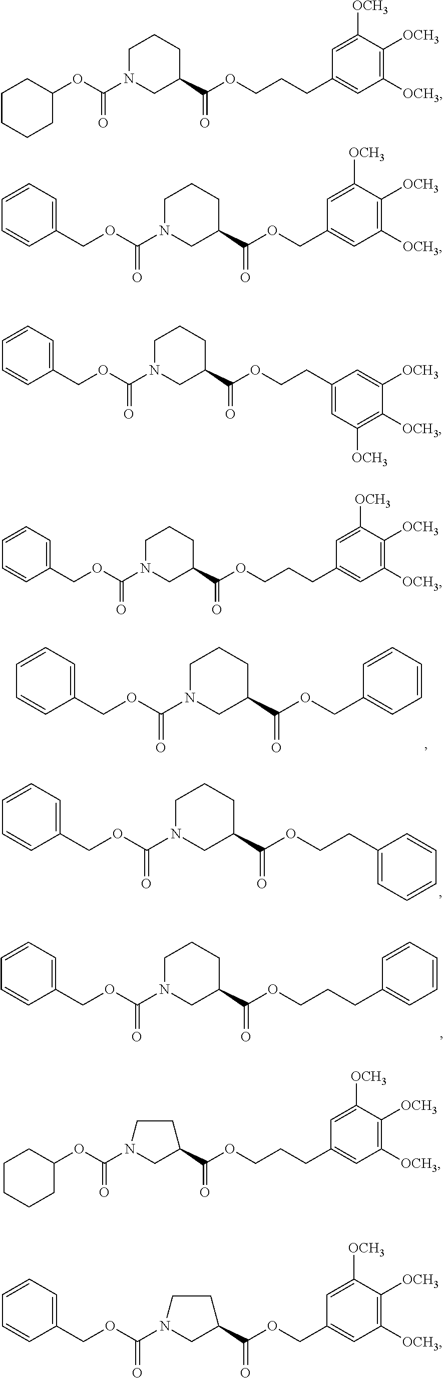

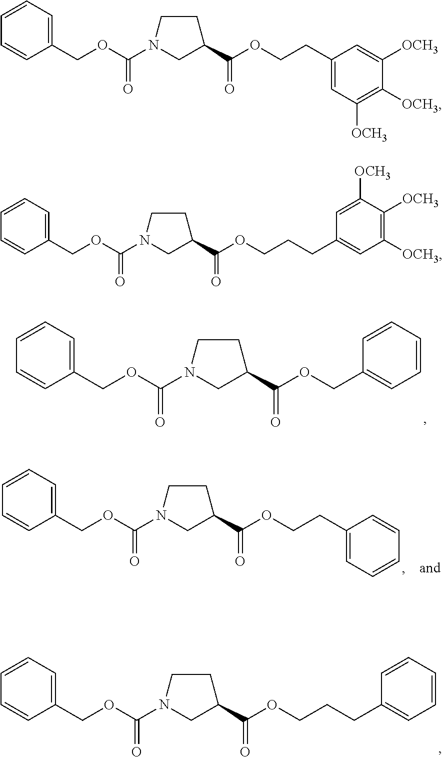

or a pharmaceutically acceptable salt thereof.

In various aspects, the compound is:

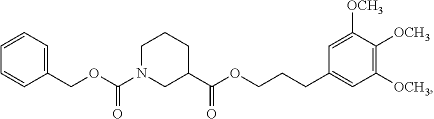 [SMILES: COc1cc(CCCOC(=O)C2CCCN(C(=O)OCc3ccccc3)C2)cc(OC)c1OC]

or a pharmaceutically acceptable salt thereof.

In various aspects, the compound is:

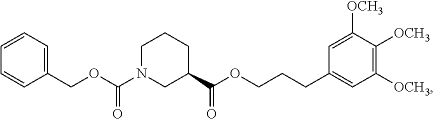 [SMILES: COc1cc(CCCOC(=O)[C@@H]2CCCN(C(=O)OCc3ccccc3)C2)cc(OC)c1OC]

or a pharmaceutically acceptable salt thereof.

In a further aspect, the compound exhibits modulation of a proteasome. In a still further aspect, the compound exhibits regulation of proteasome activity. In yet a further aspect, the compound exhibits allosteric regulation of proteasome activity.

In a further aspect, the subject is a mammal. In a still further aspect, the mammal is human.

In a further aspect, the subject has been diagnosed with a need for treatment of muscle wasting prior to the administering step. In a still further aspect, the subject is at risk for developing muscle wasting prior to the administering step.

In a further aspect, the method further comprises identifying a subject at risk for developing muscle wasting prior to the administering step. In a further aspect, the method further comprises identifying a subject in need of treatment of muscle wasting prior to the administering step.

In a further aspect, the subject is not currently diagnosed as having cancer. In a still further aspect, the subject is not currently diagnosed as having cancer during the administering step.

In a further aspect, the subject has not been diagnosed as having cancer prior to the administering step. In a still further aspect, the subject has not been diagnosed as having cancer within a time period of less than five years prior, less than three years prior, less than one year prior, less than 6 months prior, less than 3 months prior, or less than 1 month prior to the administering step.

In a further aspect, the subject has been diagnosed as having a condition selected from human immunodeficiency virus (HIV), chronic renal failure, kidney disease, chronic obstructive pulmonary disease (COPD), multiple sclerosis, cystic fibrosis, rheumatoid arthritis, advanced dementia, cachexia, or congestive heart failure (CHF) prior to the administering step. In a still further aspect, the subject has been diagnosed as having a condition selected from HIV, kidney disease, COPD, or CHF prior to the administering step.

In a further aspect, the muscle wasting is due to a condition selected from human immunodeficiency virus (HIV), chronic renal failure, kidney disease, chronic obstructive pulmonary disease (COPD), multiple sclerosis, cystic fibrosis, rheumatoid arthritis, advanced dementia, cachexia, or congestive heart failure (CHF). In a still further aspect, the muscle wasting is due to HIV, kidney disease, COPD, or CHF.

In a further aspect, the muscle wasting is due to a limb injury or immobilization.

In a further aspect, the muscle wasting is due to cancer. In a still further aspect, the muscle wasting is due to a cancer treatment (e.g., chemotherapy or radiation).

In a further aspect, the subject has cancer. Examples of cancer include, but are not limited to, pancreatic cancer, gastric cancer, esophageal cancer, stomach cancer, lung cancer, liver cancer, colon cancer, prostate cancer, non-Hodgkin lymphoma, breast cancer, a sarcoma, leukemia, and Hodgkin lymphoma. In a further aspect, the cancer is selected from pancreatic cancer and gastric cancer.

In a further aspect, the subject has a late stage cancer (e.g., stage III or stage IV). In a still further aspect, the subject has a stage III cancer. In yet a further aspect, the subject has a stage IV cancer.

In a further aspect, the subject has experienced a weight loss of 5% or more over a time period of 12 months or less prior to the administering step. In a still further aspect, the subject has experienced a weight loss of 10% or more over a time period of 12 months or less prior to the administering step.

In a further aspect, the subject has experienced a weight loss of 5% or more over a time period of 6 months or less prior to the administering step. In a still further aspect, the subject has experienced a weight loss of 10% or more over a time period of 6 months or less prior to the administering step.

In a further aspect, the subject has cancer and has experienced a weight loss of 5% or more over a time period of 12 months or less prior to the administering step. In a still further aspect, the subject has a late stage cancer and has experienced a weight loss of 5% or more over a time period of 6 months or less prior to the administering step. In yet a further aspect, the subject has a late stage cancer and has experienced a weight loss of 10% or more over a time period of 12 months or less prior to the administering step. In an even further aspect, the subject has a late stage cancer and has experienced a weight loss of 10% or more over a time period of 6 months or less prior to the administering step. In a still further aspect, the cancer is a late stage cancer. In yet a further aspect, the cancer is a late stage cancer selected from pancreatic cancer and gastric cancer.

In a further aspect, the effective amount is a therapeutically effective amount. In a still further aspect, the effective amount is a prophylactically effective amount.

### D. Kits

In various aspects, disclosed are kits comprising at least one disclosed compound or composition, and one or more of: (a) an agent known to treat muscle wasting; (b) instructions for administering the compound in connection with treating muscle wasting; and (c) instructions for treating muscle wasting.

In one aspect, disclosed are kits comprising a compound having a structure represented by a formula selected from:

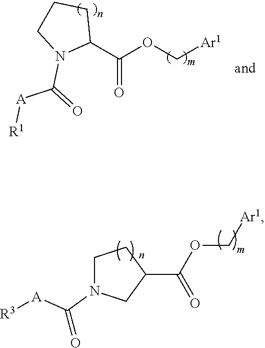

wherein n is 1 or 2; wherein m is 1, 2, or 3; wherein A is selected from —O— and —C(O)—; wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl); wherein q is 1, 2, or 3; wherein each of R^{20a} and R^{20b}, when present, is independently selected from hydrogen and C1-C4 alkyl; wherein Cy¹ is selected from cyclohexyl, a 6-membered heterocycloalkyl, and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; wherein R³ is selected from C1-C8 alkyl, (CH₂)_{q}Cy¹, and Cy¹; and wherein Ar¹ is selected from C6-aryl and pyridinyl, and is substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof, and one or more selected from: (a) an agent known to treat muscle wasting; (b) instructions for administering the compound in connection with treating muscle wasting; and (c) instructions for treating muscle wasting.

In various aspects, the agents and pharmaceutical compositions described herein can be provided in a kit. The kit can also include combinations of the agents and pharmaceutical compositions described herein.

In various aspects, the informational material can be descriptive, instructional, marketing or other material that relates to the methods described herein and/or to the use of the agents for the methods described herein. For example, the informational material may relate to the use of the agents herein to treat a subject who has, or who is at risk for developing, muscle wasting. The kits can also include paraphernalia for administering the agents of this invention to a cell (in culture or in vivo) and/or for administering a cell to a patient.

In various aspects, the informational material can include instructions for administering the pharmaceutical composition and/or cell(s) in a suitable manner to treat a human, e.g., in a suitable dose, dosage form, or mode of administration (e.g., a dose, dosage form, or mode of administration described herein). In a further aspect, the informational material can include instructions to administer the pharmaceutical composition to a suitable subject, e.g., a human having, or at risk for developing, muscle wasting.

In various aspects, the composition of the kit can include other ingredients, such as a solvent or buffer, a stabilizer, a preservative, a fragrance or other cosmetic ingredient. In such aspects, the kit can include instructions for admixing the agent and the other ingredients, or for using one or more compounds together with the other ingredients.

In a further aspect, the compound and the agent are co-formulated. In a still further aspect, the compound and the agent are co-packaged.

In a further aspect, the kit includes the agent known to treat muscle wasting. Exemplary agents known to treat muscle wasting include, but are not limited to, egestrol, somatrpoin, and serostim.

In a further aspect, the kit further comprises a plurality of dosage forms, the plurality comprising one or more doses; wherein each dose comprises an effective amount of the compound and the agent. In a still further aspect, the effective amount is a therapeutically effective amount. In yet a further aspect, the effective amount is a prophylactically effective amount. In an even further aspect, each dose of the compound and agent are co-packaged. In a still further aspect, each dose of the compound and the agent are co-formulated.

In a further aspect, the muscle wasting is due to a condition selected from human immunodeficiency virus (HIV), chronic renal failure, kidney disease, chronic obstructive pulmonary disease (COPD), multiple sclerosis, cystic fibrosis, rheumatoid arthritis, advanced dementia, cachexia, or congestive heart failure (CHF). In a still further aspect, the muscle wasting is due to HIV, kidney disease, COPD, or CHF.

In a further aspect, the muscle wasting is due to a limb injury or immobilization.

In a further aspect, the muscle wasting is due to cancer. In a still further aspect, the muscle wasting is due to a cancer treatment (e.g., chemotherapy or radiation).

### E. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Materials and Methods

a. Atomic Force Microscopy (AFM) Imaging

Cultured cells growing on dishes were scanned with a Catalyst Atomic Force Microscope (Bruker) mounted on a Nikon Ti inverted epifluorescent microscope. Nanomechanical parameters of cells were collected in the Quantitative Nanomechanical Mapping (PF-QNM) mode of the AFM. Before AFM imaging, optical images were recorded for each cell. SCANASYST-AIR probes were used for imaging after their spring constant was determined with the thermal tuning. Cell boundaries were located with peak force error (PFE) AFM images and further verified with height images. Nanomechanical parameters of cells were captured in three separate PF-QNM channels: elasticity (or stiffness—the Young's modulus), deformation and adhesion. Analysis of these parameters was performed with NanoScope Analysis software v. 1.7 (Osmulski, P. A., et al., Cancer Res. 2021 Aug. 1; 81(15):4110-4123). Force curves were fit to the Sneddon model. Mode values of the mechanical parameters for individual cells were calculated from the corresponding distribution histograms. The Texture Aspect Ratio, as a measure of the spatial isotropy or directionality of the surface texture was calculated from the square height images of cells after applying the first order flatten function (Roughness Analysis in NanoScope Analysis v. 1.7). It shows an order of linegar patterining detectable in cells (Efremov, Y. M., et al., Sci Rep 12, 4052 (2022).

b. Cell Culture

Mouse C2C12 myoblasts were purchased in American Tissue Type Collection (ATCC CRL-1772) were cultured in Dulbecco's modified Eagle's medium (DMEM) supplementec with 15% fetal bovine serum (FBS) and penicillin/streptomycin, at 37° C. and 5% CO₂. After reaching confluence the myoblasts were differentiated into myotubes by culturing for 72 hours under low-serum conditions (2% horse serum, 0.5 microgram/ml insulin. After that, model muscle atrophy was induced by the treatment with tumor necrosis factor alpha (TNFα) at 15 ng/ml for 72 hours. Vehicle (dimethylsulfoxide; DMSO) or desired compounds (B1, C3, bortezomib) were added to the culture medium together with TNFα.

Myotubes were visualized as described in Eric J. Stevenson, et al., Journal of Applied Physiology 2005 98:4, 1396-1406. All myotubes analyzed in randomly selected microscope fields, with three diameters per tube and at least 50 myotubes analyzed per experimental point.

c. In Vivo Mouse Model

The mouse model of cancer-related muscle wasting consistent of nude mice xenografted with human cultured androgen-resistant prostate cancer PC3 cells. The mice were treated intra-tumorally (IT) with the vehicle (dimethylsulfoxide; DMSO); 8 control mice) or with 10 mg/kg of compound 3. Three injections per week (Mondays, Wednesdays, and Fridays) were carried for three weeks. After that, mice were sacrificed and quadriceps muscle tissues were preserved by standard formalin fixing. Muscle tissue slices were stained with hematoxilin and eosin (H&E standard staining) to visualize muscle fibers and enable myofiber diameter measurements under a standard microscope. Myofibers for 4 control (vehicle-treated) and 4 compound 3-treated mice were measured.

d. Exemplary Proteasome-Targeting Small Molecules

The compounds shown in Table 1 below were synthesized according to the methods described in U.S. Pat. Nos. 9,918,968, 10,167,259, 11,020,383, and 11,345,659, and evaluated using the methods described herein.

TABLE 1

| No. | Structure |
|---|---|
| B1 | 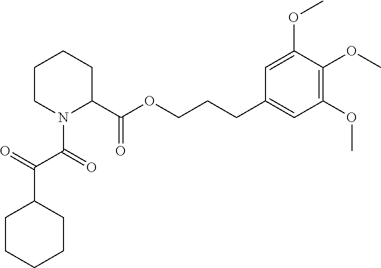 [SMILES: COc1cc(CCCOC(=O)C2CCCCN2C(=O)C(=O)C2CCCCC2)cc(OC)c1OC] |
| 2 | 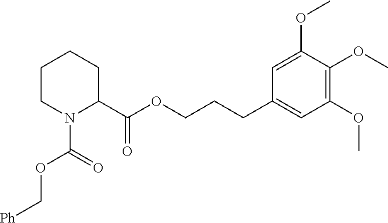 [SMILES: COc1cc(CCCOC(=O)C2CCCCN2C(=O)OCc2ccccc2)cc(OC)c1OC] |
| 3 | 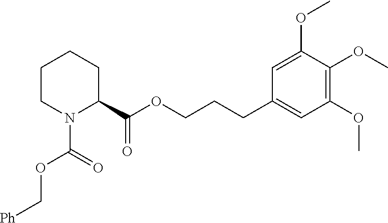 [SMILES: COc1cc(CCCOC(=O)[C@@H]2CCCCN2C(=O)OCc2ccccc2)cc(OC)c1OC] |
| 4 | 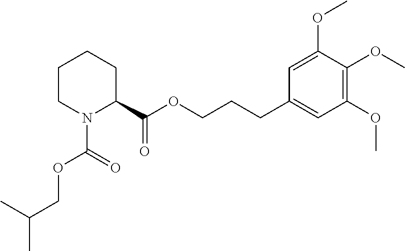 [SMILES: COc1cc(CCCOC(=O)[C@@H]2CCCCN2C(=O)OCC(C)C)cc(OC)c1OC] |
| 5 | 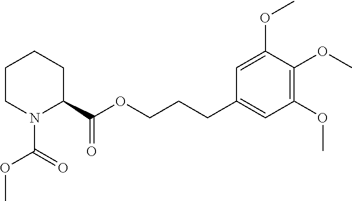 [SMILES: COC(=O)N1CCCC[C@H]1C(=O)OCCCc1cc(OC)c(OC)c(OC)c1] |
| 6 | 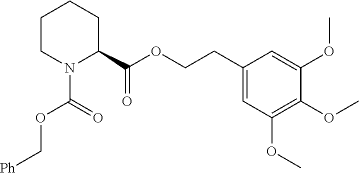 [SMILES: COc1cc(CCOC(=O)[C@@H]2CCCCN2C(=O)OCc2ccccc2)cc(OC)c1OC] |
| 7 | 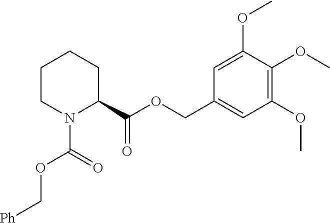 [SMILES: COc1cc(COC(=O)[C@@H]2CCCCN2C(=O)OCc2ccccc2)cc(OC)c1OC] |
| 8 | 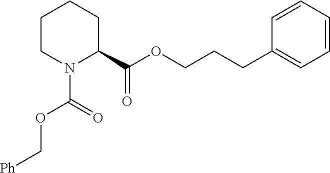 [SMILES: O=C(OCCCc1ccccc1)[C@@H]1CCCCN1C(=O)OCc1ccccc1] |
| 9 | 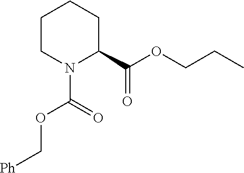 [SMILES: CCCOC(=O)[C@@H]1CCCCN1C(=O)OCc1ccccc1] |
| 10 | 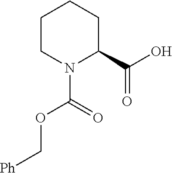 [SMILES: O=C(O)[C@@H]1CCCCN1C(=O)OCc1ccccc1] |
| 11 | 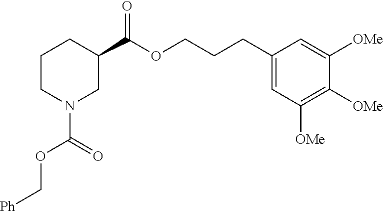 [SMILES: COc1cc(CCCOC(=O)[C@@H]2CCCN(C(=O)OCc3ccccc3)C2)cc(OC)c1OC] |
| 12 | 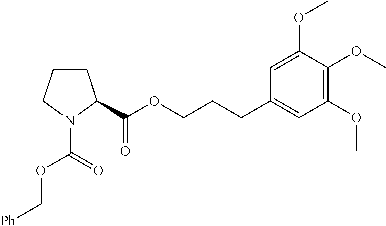 [SMILES: COc1cc(CCCOC(=O)[C@@H]2CCCN2C(=O)OCc2ccccc2)cc(OC)c1OC] |
| 13 | 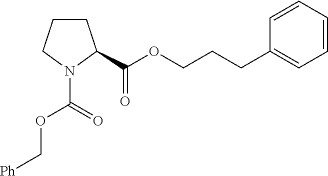 [SMILES: O=C(OCCCc1ccccc1)[C@@H]1CCCN1C(=O)OCc1ccccc1] |
| 14 | 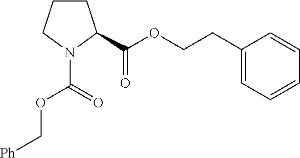 [SMILES: O=C(OCCc1ccccc1)[C@@H]1CCCN1C(=O)OCc1ccccc1] |
| 15 | 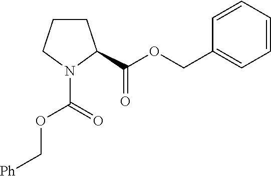 [SMILES: O=C(OCc1ccccc1)[C@@H]1CCCN1C(=O)OCc1ccccc1] |

### 2. Development of Pipecolic Ester Analogs as Proteasome Modulators

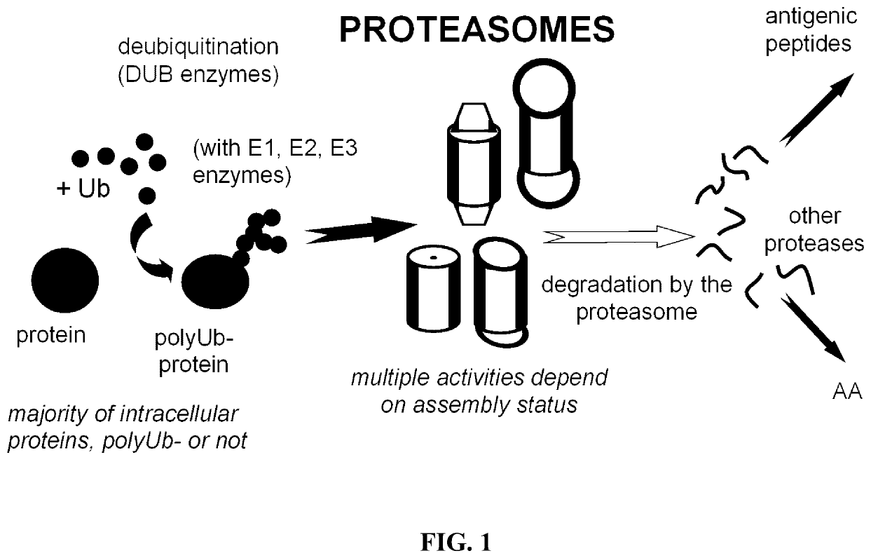

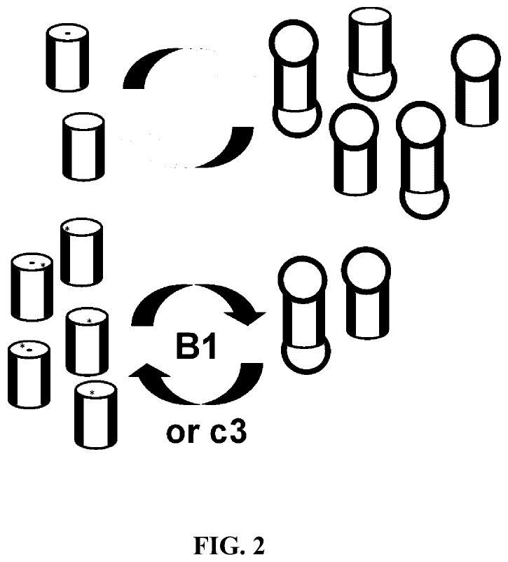

The ubiquitin-proteasome pathway is essential for intracellular proteostasis (FIG. 1). The compounds herein disclosed are indicated to be involved in the regulation of the ubiquitin-proteasome pathway in general and of proteasome activity in particular by shifting equilibrium of proteasome assemblies (FIG. 2). The compounds herein disclosed destabilize the full 26S assembly but preserve part of its activity (“single-capped” 26S) and promote formation of mixed-assemblies specialized in stress response. These small molecule modulators of the proteasome affect the protein-protein interactions in the multicomponent proteasome assembly and they guide cellular physiology to restore proteostasis.

The disclosed small molecule modulators are non-toxic and effectively prevent myotube/myofiber degeneration in a mouse model of cancer cachexia and in a cell culture model of TNFα (cachectin) dependent muscle wasting. Competitive proteasome inhibitors that by-design would ultimately abolish all proteasome-related activity are too toxic for the treatment methods described herein. For example, at least some of the known competitive drugs, including bortezomib/Velcade®, stabilize and disable the 26S proteasome, but the toxicity due to the molecular mechanism of action remains a concern. For this reason, bortezomib/Velcade was utilized as a negative control in some of the in vitro experiments described herein.

### 3. Evaluation of 20S CHT-L Activity by Pipecolic Acid Derivatives

Compounds were evaluated for proteasome activity using the 20S human housekeeping proteasome. It was activated with the Rpt5 derived 10-residue peptide that contains the C-terminal HbYX (hydrophobic-Tyr-any AA) motif activating the proteasome in trans and to some extend mimic the activation by the 19S regulatory particle (Osmulski and Gaczynska (2013) Molecular pharmacology 84: 104-113; Smith et al. (2006) J. Struct. Biol. 156: 72-83; Smith et al. (2007) Mol. Cell 27: 731-744). Proteolytic activity was monitored over time using a fluorogenic peptide substrate specific for the chymotrypsin-like active center (ChT-L, Suc-LLVY-AMC) in the presence of a concentration range of the inhibitors. In this activity assay, hydrolysis of the substrate results in release of the highly fluorescent aminomethylcoumarin fluorophore. The linear portions of the fluorescence intensity plots were used to calculate the rates of Suc-LLVY-AMC hydrolysis and an IC₅₀ (concentration of drug at which 50% of the maximum rate inhibition occurs) is determined.

Briefly, starting with the R₁-moiety, the cyclohexyl-oxoacetamide of the lead agent B1 was replaced with a benzyl carbamate to render an abridged scaffold, compound 2. The racemate compound 2 reduced the ChT-L activity of the proteasome with an IC₅₀ of 11.5 μM, which provided a great starting point to interrogate the structural requirements for activity of this new template. Next, the enantiomer of 2 was prepared, compounds (3S) starting from the corresponding chiral pipecolic acids. Gratifyingly, compound 3 was highly effective in reducing ChT-L-proteolysis by the 20S with IC₅₀ of 3.1 μM. Considering the stereochemical match with rapamycin and B1, the structure-activity relationship studies were then focused on the S-enantiomer. Replacement of the benzyl carbamate with the aliphatic mimic carbamate 4, resulted in a significant drop in activity (IC₅₀ 15.2 μM). Further reduction of size (methylcarbamate 5) completely abrogated activity. Changes in the positioning of the ester (C-2 substitution versus C-3 substitution) also resulted in a drop in activity (compound 6, IC₅₀ 14.7 μM)). In addition, shortening of the R₂-chain length from n=3 to n=2 (compound 7) or to one methylene unit (compound 8) also significantly reduced its activity. Fortunately, it was found that the trimethoxy-moieties did not significantly impact the overall potency of the ester side chain and that the free aryl group (compound 9) was found to be a potent 20S proteasome inhibitor (IC₅₀ 2.6 μM). The aryl moiety did seem to be important for activity, since the alkyl ester 10 or carboxylic acid 11 were found to be inactive.

TABLE 2

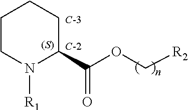

| No. | n | R₁ | R₂ | IC₅₀ (μM) |
|---|---|---|---|---|
| B1 (±) | 3 | 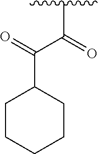 [SMILES: *C(=O)C(=O)C1CCCCC1] | 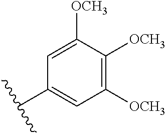 [SMILES: *c1cc(OC)c(OC)c(OC)c1] | 5.8 |
| 2 (±) | 3 | 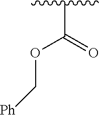 [SMILES: *C(=O)OCc1ccccc1] | 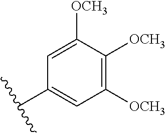 [SMILES: *c1cc(OC)c(OC)c(OC)c1] | 11.5 |
| 3 | 3 | 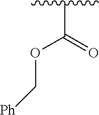 [SMILES: *C(=O)OCc1ccccc1] | 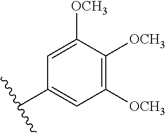 [SMILES: *c1cc(OC)c(OC)c(OC)c1] | 2.0 |
| 4 | 3 | 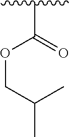 [SMILES: *C(=O)OCC(C)C] | 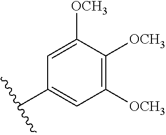 [SMILES: *c1cc(OC)c(OC)c(OC)c1] | 15.2 |
| 5 | 3 | 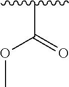 [SMILES: *C(=O)OC] | 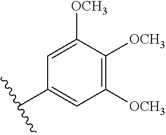 [SMILES: *c1cc(OC)c(OC)c(OC)c1] | >50 |
| 6 | 2 | 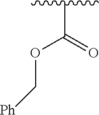 [SMILES: *C(=O)OCc1ccccc1] | 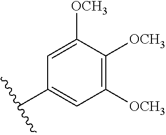 [SMILES: *c1cc(OC)c(OC)c(OC)c1] | 20.1 |
| 7 | 1 | 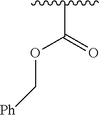 [SMILES: *C(=O)OCc1ccccc1] | 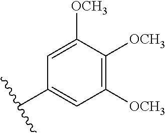 [SMILES: *c1cc(OC)c(OC)c(OC)c1] | 19.8 |
| 8 | 3 | 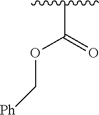 [SMILES: *C(=O)OCc1ccccc1] | 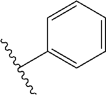 [SMILES: *c1ccccc1] | 2.6 |
| 9 | 3 | 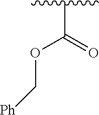 [SMILES: *C(=O)OCc1ccccc1] | H | >50 |
| 10 | 0 | 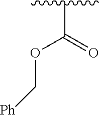 [SMILES: *C(=O)OCc1ccccc1] | H | >50 |
| 11 (ester in C-3) | 3 | 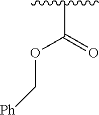 [SMILES: *C(=O)OCc1ccccc1] | 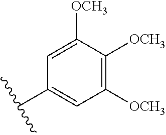 [SMILES: *c1cc(OC)c(OC)c(OC)c1] | 14.7 |

### 4. Evaluation of 20S CT-L Activity by Proline Derivatives

The ring size requirements were also investigated by replacing the pipecolic acid with a proline-type motif. The proline analogues followed the same trends as the pipecolic acid derivatives. The proline derivative 12 only had modest activity, whereas the derivative 13 exhibited good potency (IC₅₀ 6.6 μM). Reduction of the R₂-ester side chain length (compounds 14 and 15) also decreased activity, consistent with the trend seen with the pipecolic acids 7 and 8.

TABLE 3

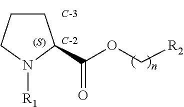

| No. | n | R₁ | R₂ | IC₅₀ (μM) |
|---|---|---|---|---|
| 12 | 3 | 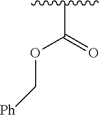 [SMILES: *C(=O)OCc1ccccc1] | 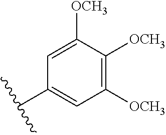 [SMILES: *c1cc(OC)c(OC)c(OC)c1] | 23.8 |
| 13 | 3 | 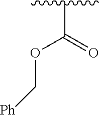 [SMILES: *C(=O)OCc1ccccc1] | 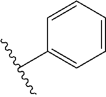 [SMILES: *c1ccccc1] | 6.6 |
| 14 | 2 | 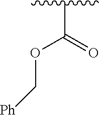 [SMILES: *C(=O)OCc1ccccc1] | 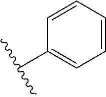 [SMILES: *c1ccccc1] | 19.2 |
| 15 | 1 | 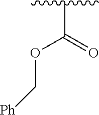 [SMILES: *C(=O)OCc1ccccc1] | 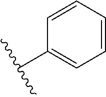 [SMILES: *c1ccccc1] | 33.5 |

### 5. In Cellulo Mouse Cell Culture Model of Myotube Degeneration

### A. Proteasome-Targeting Small Molecules Preserve Myotube Morphology

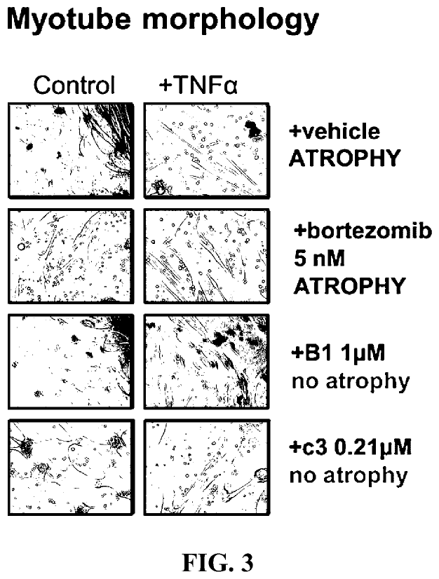

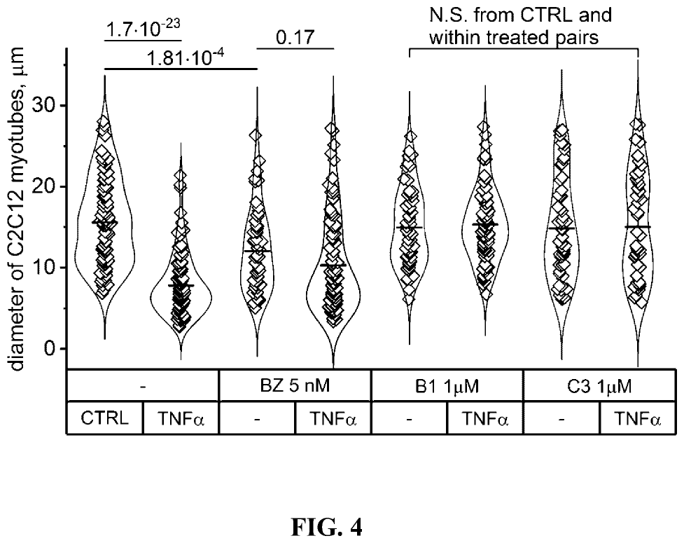

The diameters of C2C12 myotubes can be utilized to look for indications of degeneration. Comparison of cell cultures of C2C12-derived mouse myocytes of a control set and cells treated with TNFα showed the effect of these compounds on cell morphology (FIG. 3 and FIG. 4). Cells treated with only TNFα demonstrated atrophy as compared to the control. Cells treated with compound B1 or C3 showed that there was no effect on the cell morphology in the control; however, when applied alongside TNFα the compounds restored the morphogy to control-like status. The anti-cancer drug and proteasome inhibitor, bortezomib (BZ), failed to protect the myotubes from the induced wasting caused by TNFα, and actually harmed the cells as compared in the control group.

b. Proteasome-Targeting Small Molecules Preserve Organization of Cytoskeleton in Model Myotubes

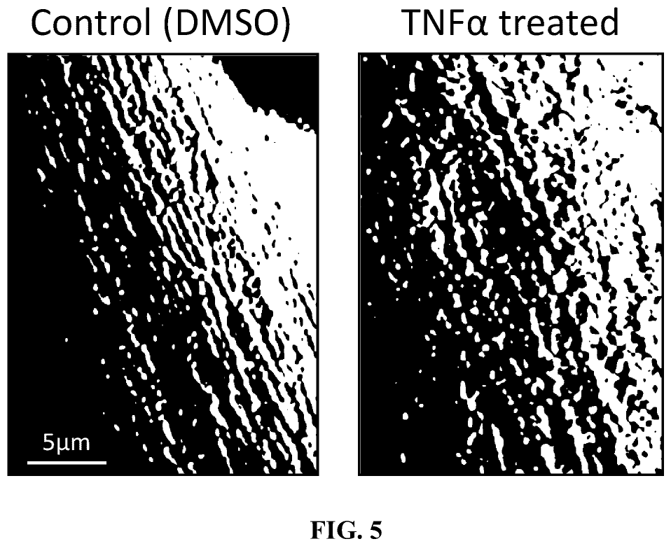

Cellular atomic force microscopy (AFM) of C2C12-derived mouse myocytes was utilized to show the effect of TNFα on the cytosketon organization of the myotube surface patterns (FIG. 5). The regularity of the pattern can be assessed by roughness analysis of the AFM images. As shown in FIG. 5, cells treated with TNFα appears less regular than that of the control cells.

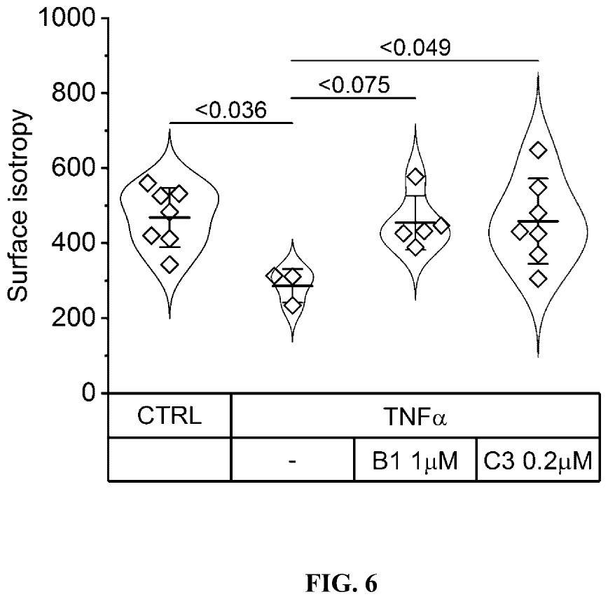

The surface isotropy parameter can quantify the regularity of the patterns observed in AFM. The higher the surface isotropy indicates a more regular pattern. Regularity of the cell surface patterning of myotubes drops significantly upon TNFα treatment (FIG. 6). As shown in FIG. 6, The patterning remains control-like (no significant difference in surface isotropy) when compounds B1 or C3 accompany the wasting-inducing agent TNFα.

c. Proteasome-Targeting Small Molecules Preserve the Mechanical Properties of Model Myotubes

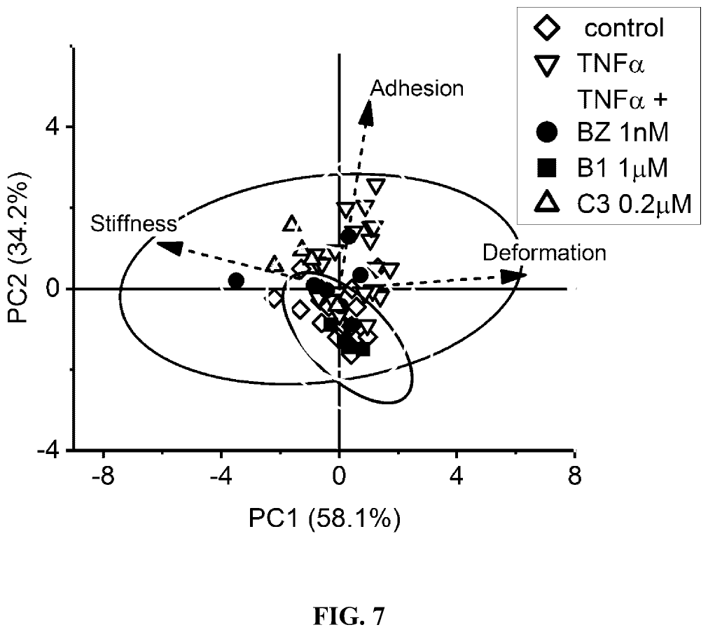

Principal component analysis (PCA) was utilized to evaluate the AFM-assessed mechanical parameters of model myotubes in C2C12 cells treated with TNFα alone or with compounds B1 or C3. Bortezomib was utilized as a further comparison (FIG. 7). As shown in FIG. 7, the mechanical parameters of “wasted” myotubes are distinct from control parameters. Compounds B1 and C3 preserve the control-like phenotypes (B1 especially well) (compare the diamonds (control) with squares (B1) and triangles (C3) ovals). However, bortezomib (BZ; circles) does not protect the phenotypes from “wasting.”

d. Insight into the Mechanism of Anti-Wasting Action of Proteasome Modulators

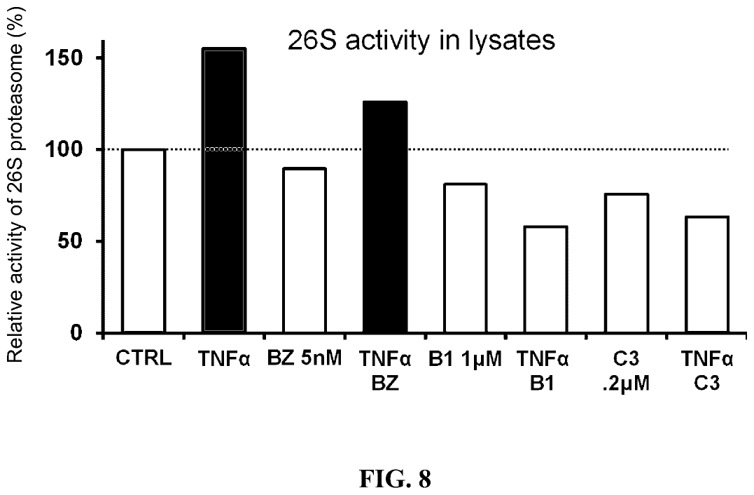

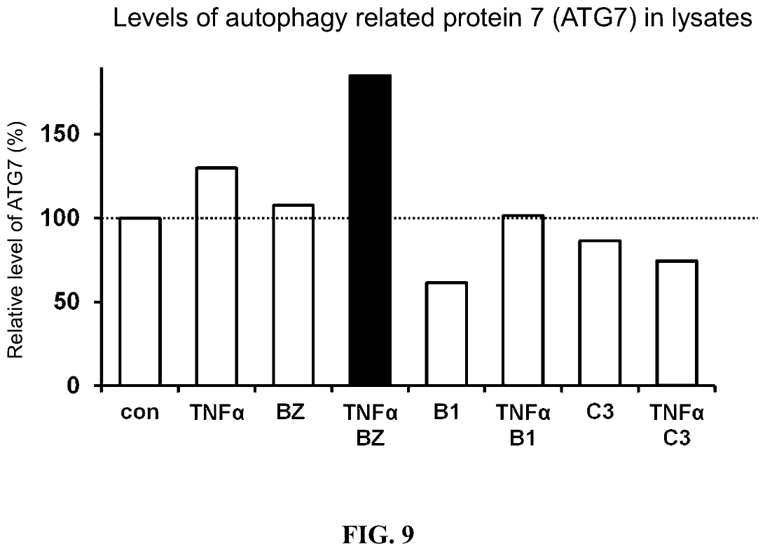

The mechanism of the anti-wasting action of proteasome modulators can be probed by measuring the 26S activity in lysates and measuring the levels of autophagy-related protein 7 (ATG7) in lysates. Excessive activity of 26S holoenzyme bears prime responsibility for degradation of muscle proteins during wasting. Treatment with B1 or C3 (but not BZ) mitigates the activity (in-gel activity assay with lysate proteins separated on 6% non-denaturing polyacrylamide gels and overlayed with model proteasome substrate Suc-LLVY-MCA to visualize activity of the gel-separated proteasomes as described in Chocron, E. S., et al., Sci. Adv. 8, eabk2252) (FIG. 8). Autophagy, alongside proteasome pathway, aggravates degeneration. Treatment with BZ upregulates autophagy in model-wasted cells, while treatment with B1 or C3 does not affect or mitigates autophagy (Western blotting) (FIG. 9).

### 6. In Vivo Mouse Model of Cancer Cachexia

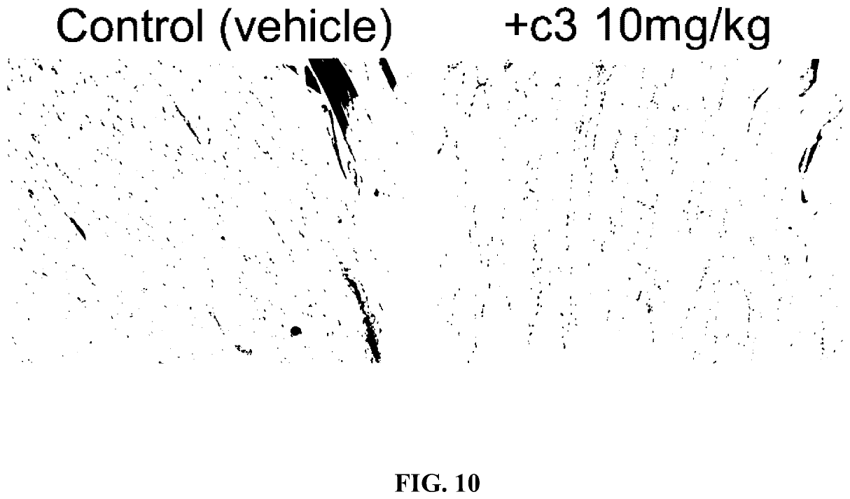

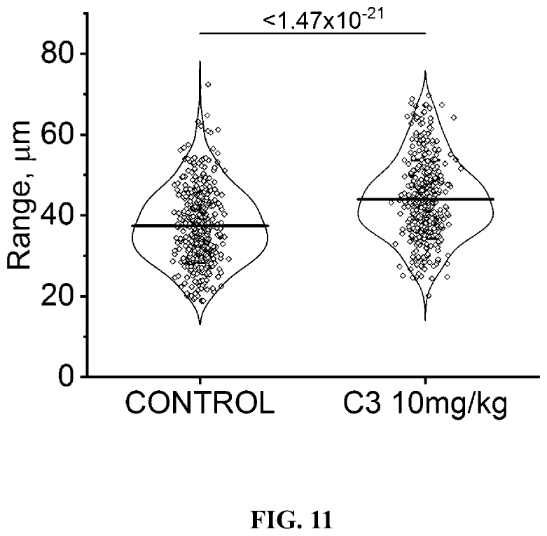

Treatment with compound C3 improved myofiber morphology in quadriceps of tumor-bearing mice (mice xenografted with aggressive human prostate cancer cells) as described above in the Materials and Methods for the in vivo mouse model. The quadriceps muscle in tumor-bearing mice treated with C3 twice a weeks for three weeks was compared to control using H&E-stained tissues slices (FIG. 10). Frequency analysis of diameters of myofibers (400 myofibers each) from tumor-bearing mice (4 each of the control-vehicle and c3 (10 mg/kg)) was performed (FIG. 11). Higher values of myofiber diameters indicate better conditions of the muscle in c3 treated mice with the difference significant at p<1.47×10⁻²¹.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of treating muscle wasting in a subject in need thereof, the method comprising administering to the subject an effective amount of a compound having a structure represented by a formula selected from: 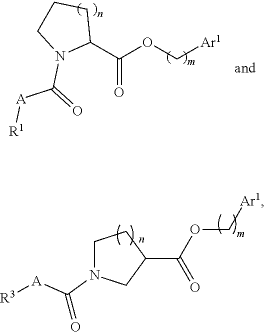
wherein n is 1 or 2;
wherein m is 1, 2, or 3;
wherein A is selected from —O— and —C(O)—;
wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl);
wherein q is 1, 2, or 3;
wherein each of R^{20a} and R^{20b}, when present, is independently selected from hydrogen and C1-C4 alkyl;
wherein Cy¹ is selected from cyclohexyl, a 6-membered heterocycloalkyl, and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino;
wherein R³ is selected from C1-C8 alkyl, (CH₂)_{q}Cy¹, and Cy¹; and
wherein Ar¹ is selected from C6-aryl and pyridinyl, and is substituted with 0, 1, 2, 3, 4, or 5 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino,
or a pharmaceutically acceptable salt thereof,
wherein the subject has not been diagnosed as having cancer prior to the administering step.

2. The method of claim 1, wherein R¹ is selected from (CH₂)_{q}Cy¹, Cy¹, and C2-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl).

3. The method of claim 1, wherein R³ is selected from C2-C8 alkyl, (CH₂)_{q}Cy¹, and Cy¹.

4. The method of claim 1, wherein Cy¹ is selected from cyclohexyl and a 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino.

5. The method of claim 1, wherein Ar¹ is selected from C6-aryl and pyridinyl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino.

6. The method of claim 1, wherein the compound has a structure represented by a formula selected from: 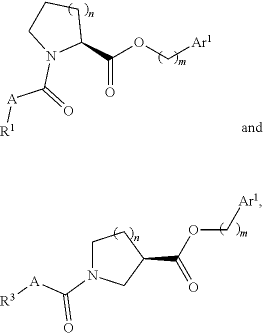
or a pharmaceutically acceptable salt thereof.

7. The method of claim 1, wherein the compound has a structure represented by a formula selected from: 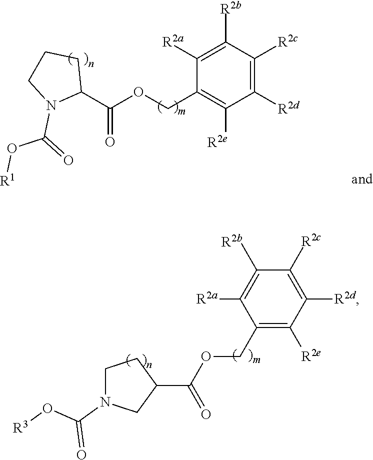
wherein n is 1 or 2;
wherein m is 1, 2, or 3;
wherein R¹ is selected from (CH₂)_{q}Cy¹, Cy¹, and C1-C8 acyclic alkyl substituted with 0, 1, 2, or 3 groups independently selected from halogen, —CN, —NH₂, —NO₂, —OH, —C(O)NR^{20a}R^{20b}, —CO₂H, and —CO₂(C1-C4 alkyl);
wherein q, when present, is 1, 2, or 3;
wherein each of R^{20a} and R^{20b}, when present, is independently selected from hydrogen and C1-C4 alkyl;
wherein Cy¹ is selected from cyclohexyl and 6-membered monocyclic aryl, and is substituted with 0, 1, 2, or 3 groups independently selected from halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; and
wherein each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino; and
wherein R³, when present, is selected from C1-C8 alkyl, (CH₂)_{q}Cy¹, and Cy¹,
or a pharmaceutically acceptable salt thereof.

8. The method of claim 1, wherein the compound has a structure represented by a formula: 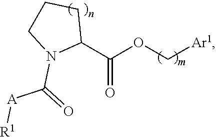
or a pharmaceutically acceptable salt thereof.

9. The method of claim 7, wherein the compound has a structure represented by a formula: 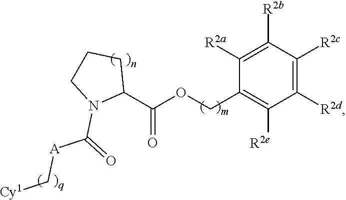
wherein each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino, or a pharmaceutically acceptable salt thereof.

10. The method of claim 7, wherein the compound has a structure selected from: 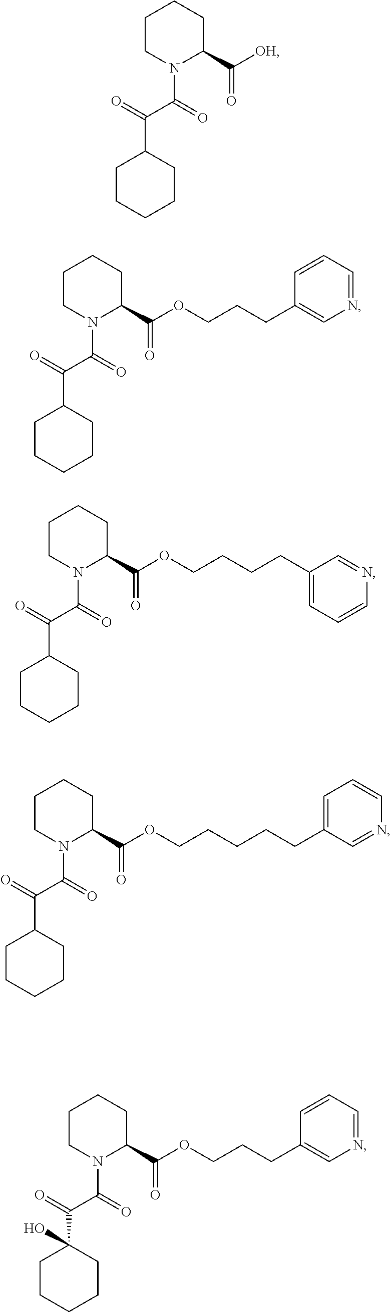 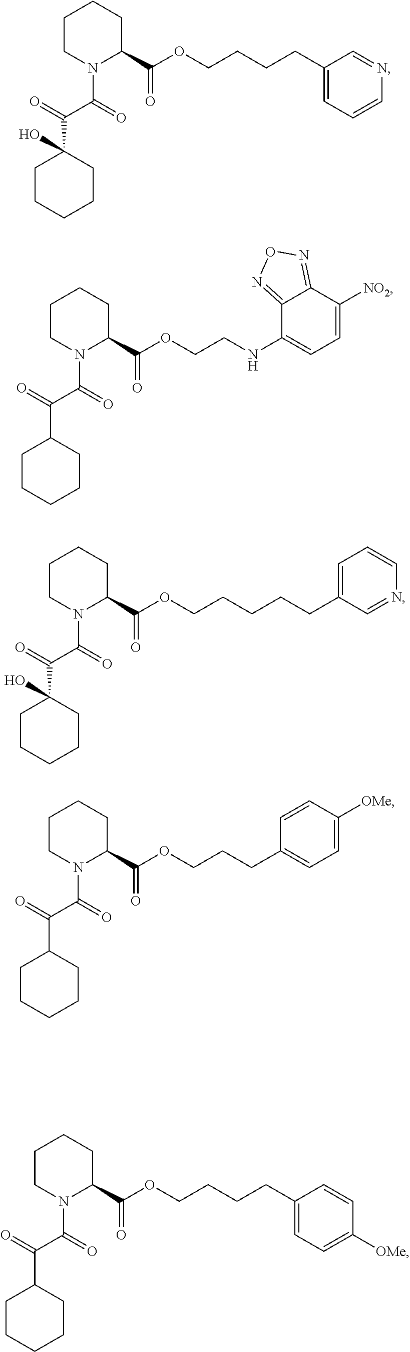 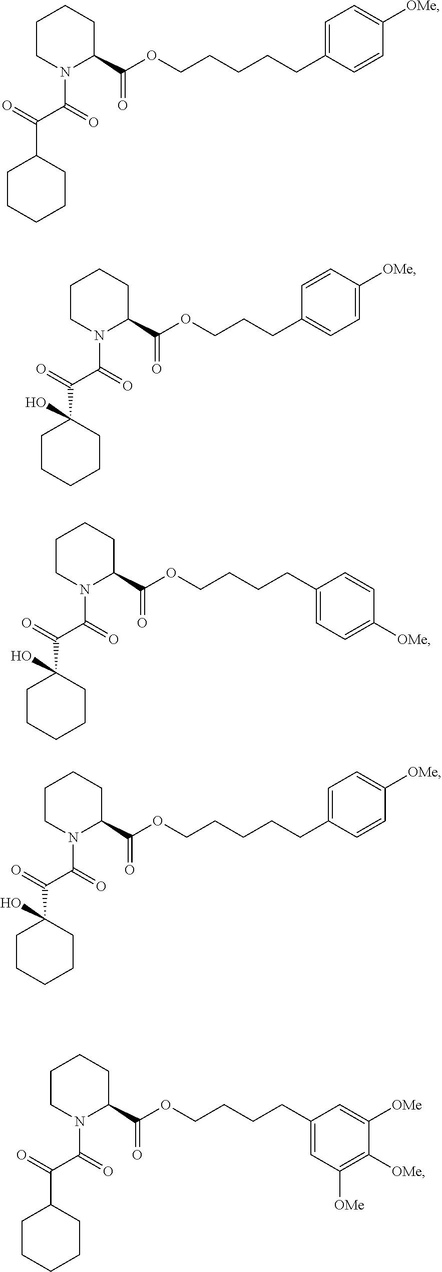 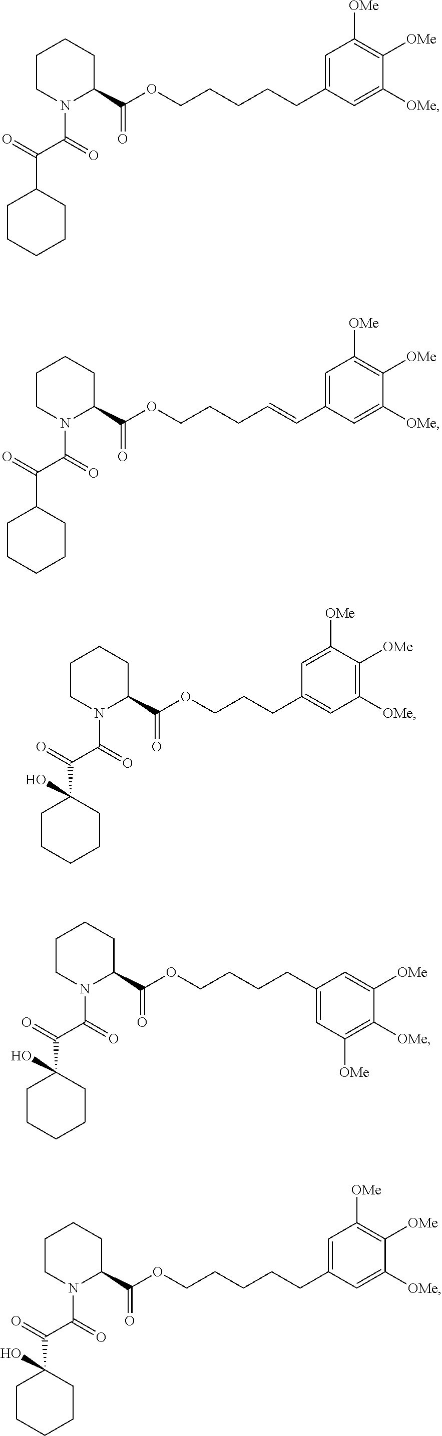 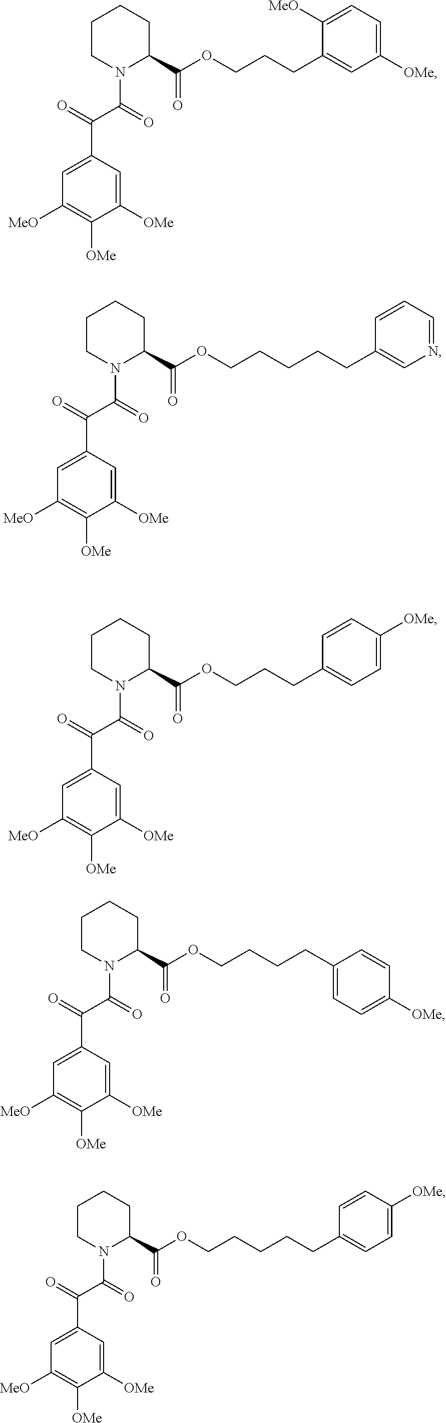 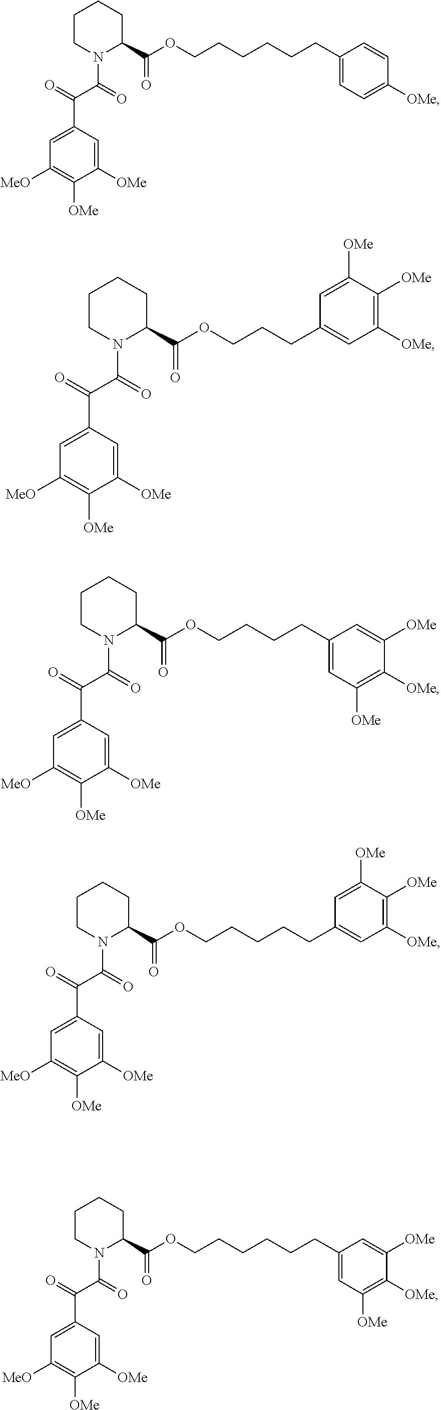 [SMILES: COc1ccc(CCCCCCOC(=O)[C@@H]2CCCCN2C(=O)C(=O)c2cc(OC)c(OC)c(OC)c2)cc1 ; COc1cc(CCCOC(=O)[C@@H]2CCCCN2C(=O)C(=O)c2cc(OC)c(OC)c(OC)c2)cc(OC)c1OC ; COc1cc(CCCCOC(=O)[C@@H]2CCCCN2C(=O)C(=O)c2cc(OC)c(OC)c(OC)c2)cc(OC)c1OC ; COc1cc(CCCCCOC(=O)[C@@H]2CCCCN2C(=O)C(=O)c2cc(OC)c(OC)c(OC)c2)cc(OC)c1OC ; COc1cc(CCCCCCOC(=O)[C@@H]2CCCCN2C(=O)C(=O)c2cc(OC)c(OC)c(OC)c2)cc(OC)c1OC] 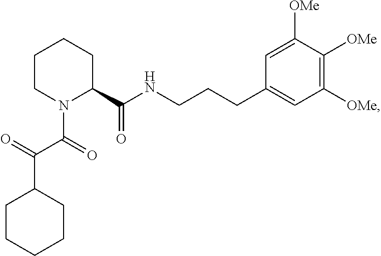 [SMILES: COc1cc(CCCNC(=O)[C@@H]2CCCCN2C(=O)C(=O)C2CCCCC2)cc(OC)c1OC] 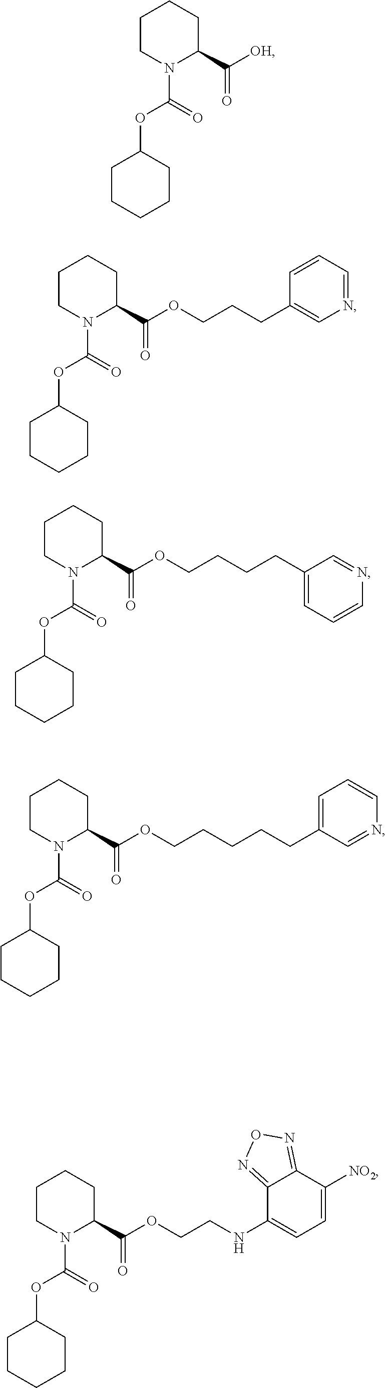 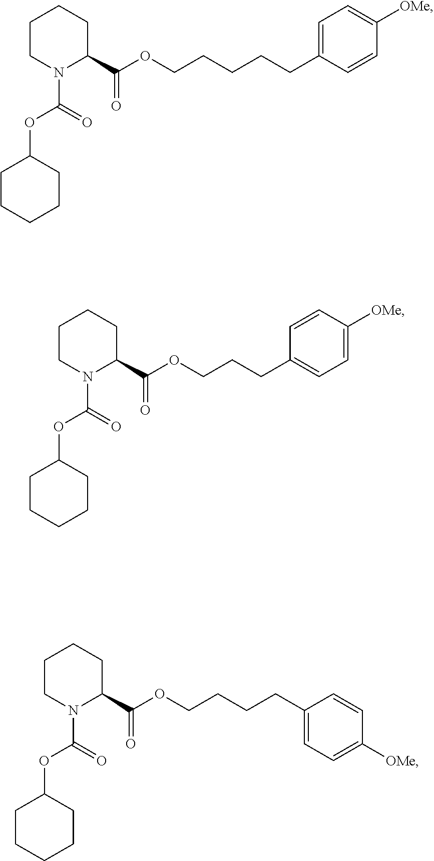 [SMILES: COc1ccc(CCCCCOC(=O)[C@@H]2CCCCN2C(=O)OC2CCCCC2)cc1 ; COc1ccc(CCCOC(=O)[C@@H]2CCCCN2C(=O)OC2CCCCC2)cc1 ; COc1ccc(CCCCOC(=O)[C@@H]2CCCCN2C(=O)OC2CCCCC2)cc1] 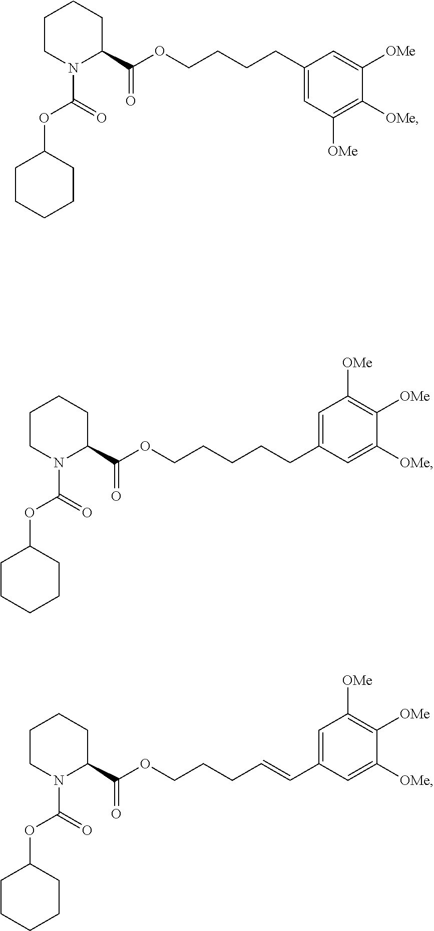 [SMILES: COc1cc(CCCCOC(=O)[C@@H]2CCCCN2C(=O)OC2CCCCC2)cc(OC)c1OC ; COc1cc(CCCCCOC(=O)[C@@H]2CCCCN2C(=O)OC2CCCCC2)cc(OC)c1OC ; COc1cc(/C=C/CCCOC(=O)[C@@H]2CCCCN2C(=O)OC2CCCCC2)cc(OC)c1OC] 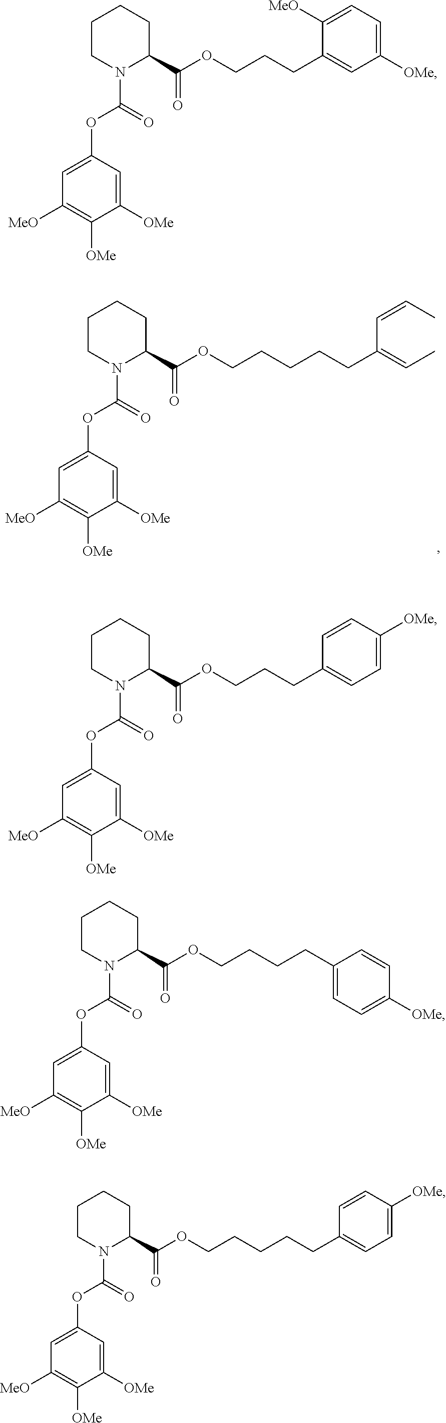 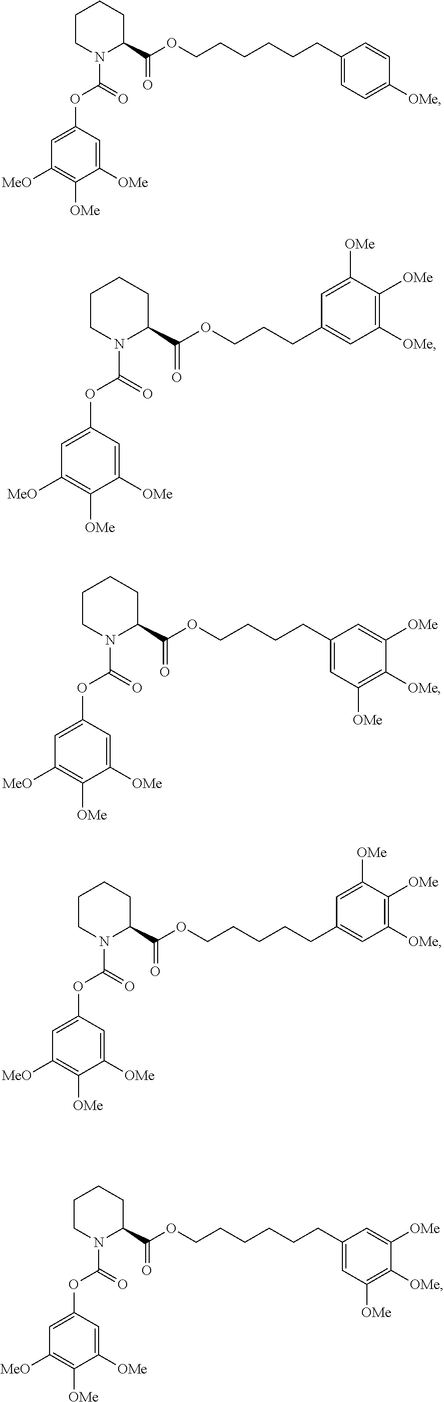 [SMILES: COc1ccc(CCCCCCOC(=O)[C@@H]2CCCCN2C(=O)Oc2cc(OC)c(OC)c(OC)c2)cc1 ; COc1cc(CCCOC(=O)[C@@H]2CCCCN2C(=O)Oc2cc(OC)c(OC)c(OC)c2)cc(OC)c1OC ; COc1cc(CCCCOC(=O)[C@@H]2CCCCN2C(=O)Oc2cc(OC)c(OC)c(OC)c2)cc(OC)c1OC ; COc1cc(CCCCCOC(=O)[C@@H]2CCCCN2C(=O)Oc2cc(OC)c(OC)c(OC)c2)cc(OC)c1OC ; COc1cc(CCCCCCOC(=O)[C@@H]2CCCCN2C(=O)Oc2cc(OC)c(OC)c(OC)c2)cc(OC)c1OC] 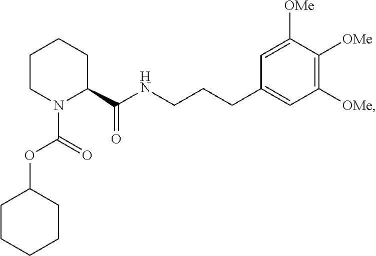 [SMILES: COc1cc(CCCNC(=O)[C@@H]2CCCCN2C(=O)OC2CCCCC2)cc(OC)c1OC] 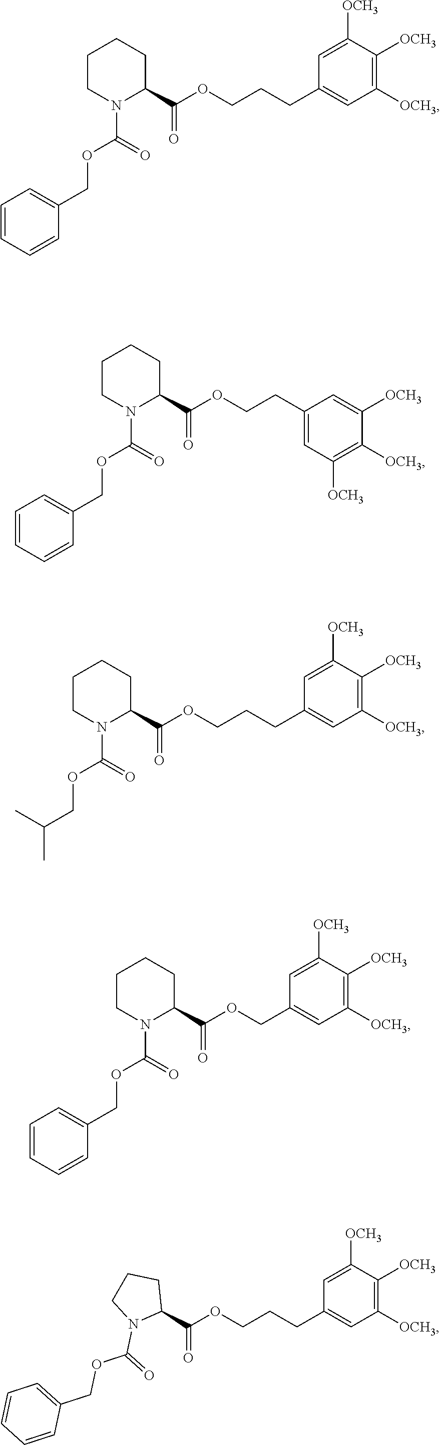 [SMILES: COc1cc(CCCOC(=O)[C@@H]2CCCCN2C(=O)OCc2ccccc2)cc(OC)c1OC ; COc1cc(CCOC(=O)[C@@H]2CCCCN2C(=O)OCc2ccccc2)cc(OC)c1OC ; COc1cc(CCCOC(=O)[C@@H]2CCCCN2C(=O)OCC(C)C)cc(OC)c1OC ; COc1cc(COC(=O)[C@@H]2CCCCN2C(=O)OCc2ccccc2)cc(OC)c1OC ; COc1cc(CCCOC(=O)[C@@H]2CCCN2C(=O)OCc2ccccc2)cc(OC)c1OC] 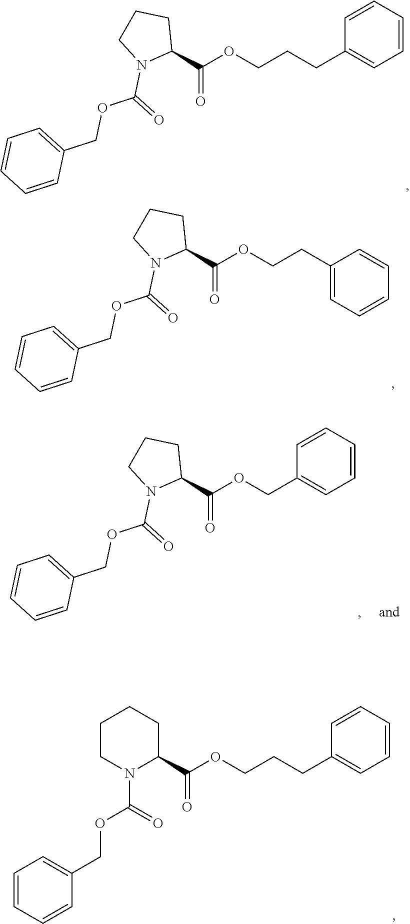 [SMILES: O=C(OCCCc1ccccc1)[C@@H]1CCCN1C(=O)OCc1ccccc1 ; O=C(OCCc1ccccc1)[C@@H]1CCCN1C(=O)OCc1ccccc1 ; O=C(OCc1ccccc1)[C@@H]1CCCN1C(=O)OCc1ccccc1 ; O=C(OCCCc1ccccc1)[C@@H]1CCCCN1C(=O)OCc1ccccc1]
or a pharmaceutically acceptable salt thereof.

11. The method of claim 7, wherein the compound is selected from: 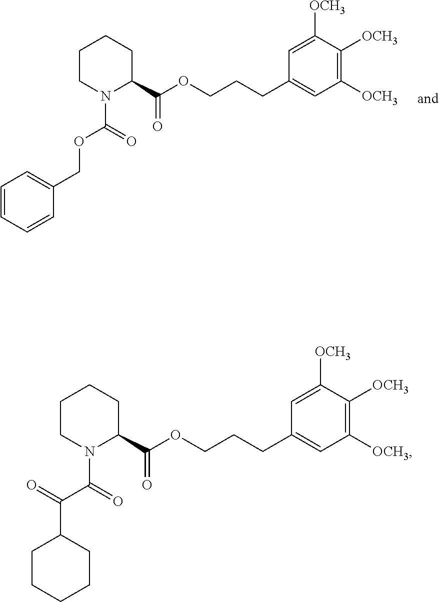 [SMILES: COc1cc(CCCOC(=O)[C@@H]2CCCCN2C(=O)OCc2ccccc2)cc(OC)c1OC ; COc1cc(CCCOC(=O)[C@@H]2CCCCN2C(=O)C(=O)C2CCCCC2)cc(OC)c1OC]
or a pharmaceutically acceptable salt thereof.

12. The method of claim 1, wherein the compound has a structure: 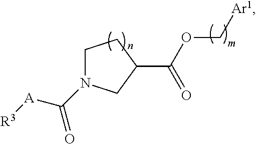
or a pharmaceutically acceptable salt thereof.

13. The method of claim 11, wherein the compound has a structure represented by a formula: 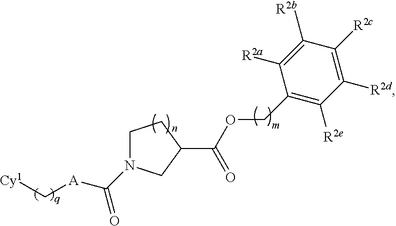
wherein each of R^{2a}, R^{2b}, R^{2c}, R^{2d}, and R^{2e} is independently selected from hydrogen, halogen, —CN, —NH₂, —NO₂, —OH, C1-C4 alkyl, C1-C4 haloalkyl, C1-C4 hydroxyalkyl, C1-C4 alkoxy, C1-C4 haloalkoxy, C1-C4 alkylamino, and (C1-C4)(C1-C4) dialkylamino,
or a pharmaceutically acceptable salt thereof.

14. The method of claim 11, wherein the compound has a structure selected from: 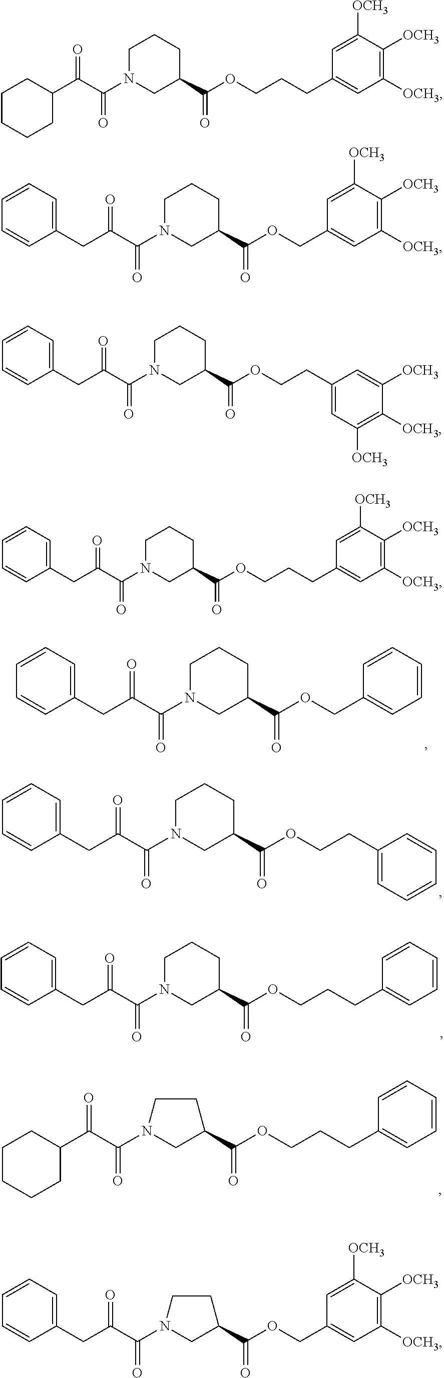 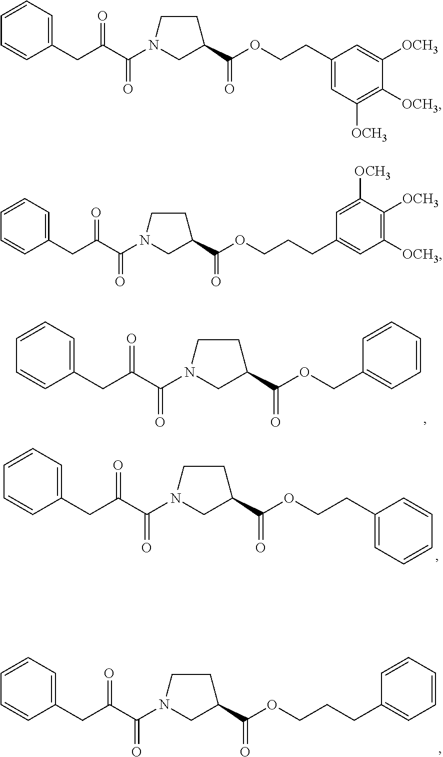 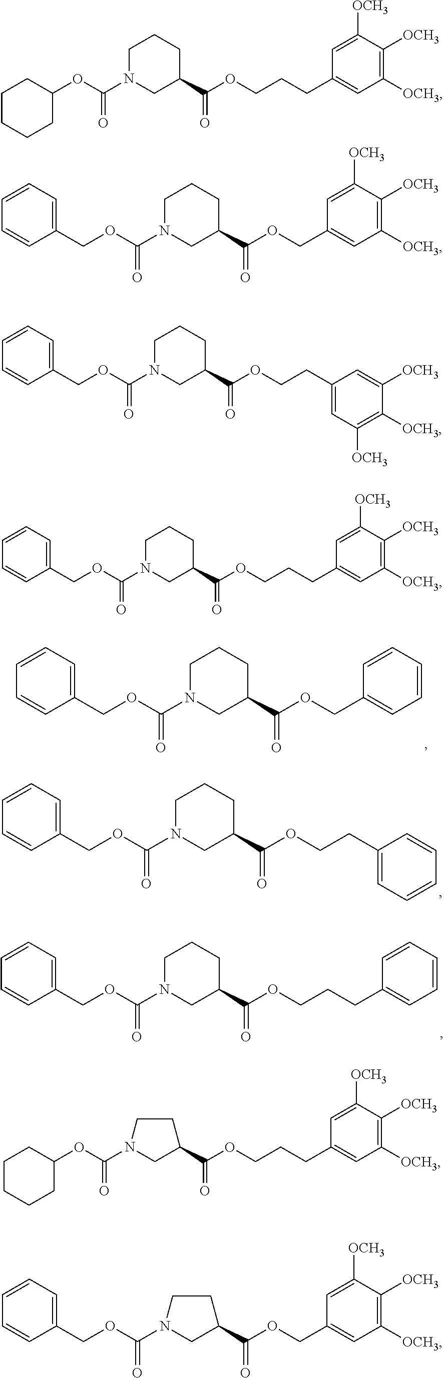 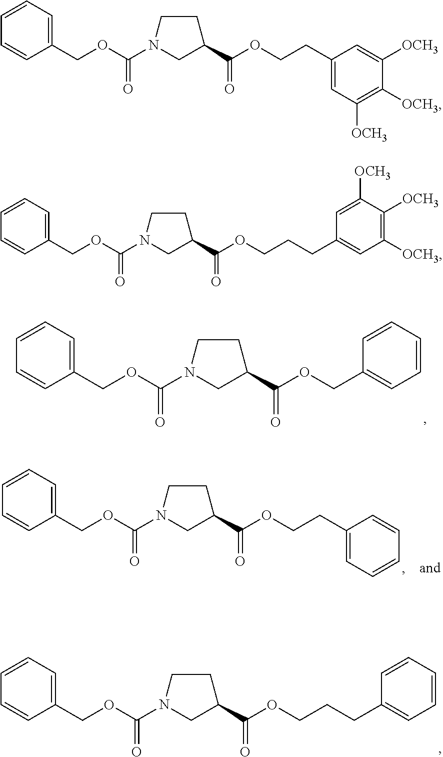
or a pharmaceutically acceptable salt thereof.

15. The method of claim 11, wherein the compound is: 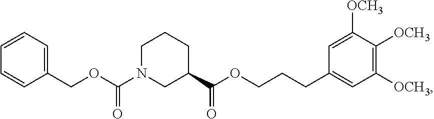 [SMILES: COc1cc(CCCOC(=O)[C@@H]2CCCN(C(=O)OCc3ccccc3)C2)cc(OC)c1OC]
or a pharmaceutically acceptable salt thereof.

16. The method of claim 1, wherein the subject has not been diagnosed as having cancer within a time period of less than one year prior to the administering step.

17. The method of claim 1, wherein the subject is not currently undergoing treatment for cancer.

18. The method of claim 1, wherein the subject is not currently diagnosed as having cancer.

19. The method of claim 1, wherein the subject has been diagnosed as having a condition selected from human immunodeficiency virus (HIV), chronic renal failure, kidney disease, chronic obstructive pulmonary disease (COPD), multiple sclerosis, cystic fibrosis, rheumatoid arthritis, advanced dementia, cachexia, or congestive heart failure (CHF) prior to the administering step.

20. The method of claim 1, wherein administering is via oral administration.
